SEQUENCE LISTING (412 sequences)

SEQ ID NO: 1 - RNA, 100 nt, synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 2 - RNA, 100 nt, synthetic construct; identical to SEQ ID NO: 1
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 3 - RNA, 20 nt, synthetic construct
  source 1..20: mol_type = other RNA
agagcaacagtgctgtggcc

SEQ ID NO: 4 - RNA, 20 nt, synthetic construct
  source 1..20: mol_type = other RNA
  misc_feature 1..4: note = modified with 2'-O-methyl phosphorothioate
agagcaacagtgctgtggcc

SEQ ID NO: 5 - RNA, 100 nt, synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 6 - RNA, 100 nt, synthetic construct; identical to SEQ ID NO: 5
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 7 - RNA, 20 nt, synthetic construct
  source 1..20: mol_type = other RNA
gctactctctctttctggcc

SEQ ID NO: 8 - RNA, 20 nt, synthetic construct
  source 1..20: mol_type = other RNA
  misc_feature 1..4: note = modified with 2'-O-methyl phosphorothioate
gctactctctctttctggcc

SEQ ID NO: 9 - RNA, 100 nt, synthetic construct
  source 1..100: mol_type = other RNA
gctttggtcccattggtcgcgttttagagctagaaatagcaagttaaaataaggctagtccgttatcaacttgaaaaagtggcaccgagtcggtgctttt

SEQ ID NO: 10 - RNA, 100 nt, synthetic construct; identical to SEQ ID NO: 9
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 11 - RNA, 20 nt, synthetic construct
  source 1..20: mol_type = other RNA
gctttggtcccattggtcgc

SEQ ID NO: 12 - RNA, 20 nt, synthetic construct
  source 1..20: mol_type = other RNA
  misc_feature 1..4: note = modified with 2'-O-methyl phosphorothioate
gctttggtcccattggtcgc

SEQ ID NO: 13 - DNA, 23 bp, synthetic construct
  source 1..23: mol_type = other DNA
agagcaacagtgctgtggcctgg

SEQ ID NO: 14 - DNA, 20 bp, synthetic construct
  source 1..20: mol_type = other DNA
agagcaacagtgctgtggcc

SEQ ID NO: 15 - DNA, 23 bp, synthetic construct
  source 1..23: mol_type = other DNA
gctactctctctttctggcctgg

SEQ ID NO: 16 - DNA, 20 bp, synthetic construct
  source 1..20: mol_type = other DNA
gctactctctctttctggcc

SEQ ID NO: 17 - DNA, 23 bp, synthetic construct
  source 1..23: mol_type = other DNA
gctttggtcccattggtcgcggg

SEQ ID NO: 18 - DNA, 20 bp, synthetic construct
  source 1..20: mol_type = other DNA
gctttggtcccattggtcgc

SEQ ID NO: 19 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 20 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 21 - RNA, 114 nt, synthetic construct
  source 1..114: mol_type = other RNA
  misc_feature 18..30: note = n is a, g, c,u, or absent
  misc_feature 108..114: note = u or absent
nnnnnnnnnnnnnnnnnnnnnnnnnnnnnngttttagagctagaaatagcaagttaaaataaggctagtccgttatcaacttgaaaaagtggcaccgagtcggtgctttttttt

SEQ ID NO: 22 - protein, 1368 aa, synthetic construct [sequence omitted]

SEQ ID NO: 23 - RNA, 100 nt, synthetic construct
  source 1..100: mol_type = other RNA
tacccaaaattcgaccttttgttttagagctagaaatagcaagttaaaataaggctagtccgttatcaacttgaaaaagtggcaccgagtcggtgctttt

SEQ ID NO: 24 - RNA, 100 nt, synthetic construct; identical to SEQ ID NO: 23
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 25 - RNA, 20 nt, synthetic construct
  source 1..20: mol_type = other RNA
tacccaaaattcgacctttt

SEQ ID NO: 26 - RNA, 20 nt, synthetic construct
  source 1..20: mol_type = other RNA
  misc_feature 1..4: note = modified with 2'-O-methyl phosphorothioate
tacccaaaattcgacctttt

SEQ ID NO: 27 - RNA, 100 nt, synthetic construct
  source 1..100: mol_type = other RNA
ccccgaaaaggtcgaattttgttttagagctagaaatagcaagttaaaataaggctagtccgttatcaacttgaaaaagtggcaccgagtcggtgctttt

SEQ ID NO: 28 - RNA, 100 nt, synthetic construct; identical to SEQ ID NO: 27
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 29 - RNA, 20 nt, synthetic construct
  source 1..20: mol_type = other RNA
ccccgaaaaggtcgaatttt

SEQ ID NO: 30 - RNA, 20 nt, synthetic construct
  source 1..20: mol_type = other RNA
  misc_feature 1..4: note = modified with 2'-O-methyl phosphorothioate
ccccgaaaaggtcgaatttt

SEQ ID NO: 31 - RNA, 100 nt, synthetic construct
  source 1..100: mol_type = other RNA
gggtattattgatgctattcgttttagagctagaaatagcaagttaaaataaggctagtccgttatcaacttgaaaaagtggcaccgagtcggtgctttt

SEQ ID NO: 32 - RNA, 100 nt, synthetic construct; identical to SEQ ID NO: 31
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 33 - RNA, 20 nt, synthetic construct
  source 1..20: mol_type = other RNA
gggtattattgatgctattc

SEQ ID NO: 34 - RNA, 20 nt, synthetic construct
  source 1..20: mol_type = other RNA
  misc_feature 1..4: note = modified with 2'-O-methyl phosphorothioate
gggtattattgatgctattc

SEQ ID NO: 35 - RNA, 100 nt, synthetic construct
  source 1..100: mol_type = other RNA
gatctgcggcagcttgcttagttttagagctagaaatagcaagttaaaataaggctagtccgttatcaacttgaaaaagtggcaccgagtcggtgctttt

SEQ ID NO: 36 - RNA, 100 nt, synthetic construct; identical to SEQ ID NO: 35
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 37 - RNA, 20 nt, synthetic construct
  source 1..20: mol_type = other RNA
gatctgcggcagcttgctta

SEQ ID NO: 38 - RNA, 20 nt, synthetic construct
  source 1..20: mol_type = other RNA
  misc_feature 1..4: note = modified with 2'-O-methyl phosphorothioate
gatctgcggcagcttgctta

SEQ ID NO: 39 - RNA, 100 nt, synthetic construct
  source 1..100: mol_type = other RNA
agcaagctgccgcagatcgcgttttagagctagaaatagcaagttaaaataaggctagtccgttatcaacttgaaaaagtggcaccgagtcggtgctttt

SEQ ID NO: 40 - RNA, 100 nt, synthetic construct; identical to SEQ ID NO: 39
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 41 - RNA, 20 nt, synthetic construct
  source 1..20: mol_type = other RNA
agcaagctgccgcagatcgc

SEQ ID NO: 42 - RNA, 20 nt, synthetic construct
  source 1..20: mol_type = other RNA
  misc_feature 1..4: note = modified with 2'-O-methyl phosphorothioate
agcaagctgccgcagatcgc

SEQ ID NO: 43 - RNA, 100 nt, synthetic construct
  source 1..100: mol_type = other RNA
tgccgcagatcgcaggaccggttttagagctagaaatagcaagttaaaataaggctagtccgttatcaacttgaaaaagtggcaccgagtcggtgctttt

SEQ ID NO: 44 - RNA, 100 nt, synthetic construct; identical to SEQ ID NO: 43
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 45 - RNA, 20 nt, synthetic construct
  source 1..20: mol_type = other RNA
tgccgcagatcgcaggaccg

SEQ ID NO: 46 - RNA, 20 nt, synthetic construct
  source 1..20: mol_type = other RNA
  misc_feature 1..4: note = modified with 2'-O-methyl phosphorothioate
tgccgcagatcgcaggaccg

SEQ ID NO: 47 - RNA, 100 nt, synthetic construct
  source 1..100: mol_type = other RNA
ccaatcgagtgcaacttaacgttttagagctagaaatagcaagttaaaataaggctagtccgttatcaacttgaaaaagtggcaccgagtcggtgctttt

SEQ ID NO: 48 - RNA, 100 nt, synthetic construct; identical to SEQ ID NO: 47
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 49 - RNA, 20 nt, synthetic construct
  source 1..20: mol_type = other RNA
ccaatcgagtgcaacttaac

SEQ ID NO: 50 - RNA, 20 nt, synthetic construct
  source 1..20: mol_type = other RNA
  misc_feature 1..4: note = modified with 2'-O-methyl phosphorothioate
ccaatcgagtgcaacttaac

SEQ ID NO: 51 - RNA, 100 nt, synthetic construct
  source 1..100: mol_type = other RNA
ccggttaagttgcactcgatgttttagagctagaaatagcaagttaaaataaggctagtccgttatcaacttgaaaaagtggcaccgagtcggtgctttt

SEQ ID NO: 52 - RNA, 100 nt, synthetic construct; identical to SEQ ID NO: 51
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 53 - RNA, 20 nt, synthetic construct
  source 1..20: mol_type = other RNA
ccggttaagttgcactcgat

SEQ ID NO: 54 - RNA, 20 nt, synthetic construct
  source 1..20: mol_type = other RNA
  misc_feature 1..4: note = modified with 2'-O-methyl phosphorothioate
ccggttaagttgcactcgat

SEQ ID NO: 55 - RNA, 100 nt, synthetic construct
  source 1..100: mol_type = other RNA
cggttaagttgcactcgattgttttagagctagaaatagcaagttaaaataaggctagtccgttatcaacttgaaaaagtggcaccgagtcggtgctttt

SEQ ID NO: 56 - RNA, 100 nt, synthetic construct; identical to SEQ ID NO: 55
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 57 - RNA, 20 nt, synthetic construct
  source 1..20: mol_type = other RNA
cggttaagttgcactcgatt

SEQ ID NO: 58 - RNA, 20 nt, synthetic construct
  source 1..20: mol_type = other RNA
  misc_feature 1..4: note = modified with 2'-O-methyl phosphorothioate
cggttaagttgcactcgatt

SEQ ID NO: 59 - RNA, 100 nt, synthetic construct
  source 1..100: mol_type = other RNA
gttgcactcgattgggacaggttttagagctagaaatagcaagttaaaataaggctagtccgttatcaacttgaaaaagtggcaccgagtcggtgctttt

SEQ ID NO: 60 - RNA, 100 nt, synthetic construct; identical to SEQ ID NO: 59
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 61 - RNA, 20 nt, synthetic construct
  source 1..20: mol_type = other RNA
gttgcactcgattgggacag

SEQ ID NO: 62 - RNA, 20 nt, synthetic construct
  source 1..20: mol_type = other RNA
  misc_feature 1..4: note = modified with 2'-O-methyl phosphorothioate
gttgcactcgattgggacag

SEQ ID NO: 63 - RNA, 100 nt, synthetic construct
  source 1..100: mol_type = other RNA
ttgcactcgattgggacagtgttttagagctagaaatagcaagttaaaataaggctagtccgttatcaacttgaaaaagtggcaccgagtcggtgctttt

SEQ ID NO: 64 - RNA, 100 nt, synthetic construct; identical to SEQ ID NO: 63
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 65 - RNA, 20 nt, synthetic construct
  source 1..20: mol_type = other RNA
ttgcactcgattgggacagt

SEQ ID NO: 66 - RNA, 20 nt, synthetic construct
  source 1..20: mol_type = other RNA
  misc_feature 1..4: note = modified with 2'-O-methyl phosphorothioate
ttgcactcgattgggacagt

SEQ ID NO: 67 - RNA, 100 nt, synthetic construct
  source 1..100: mol_type = other RNA
gcacctcttgccttacggcagttttagagctagaaatagcaagttaaaataaggctagtccgttatcaacttgaaaaagtggcaccgagtcggtgctttt

SEQ ID NO: 68 - RNA, 100 nt, synthetic construct; identical to SEQ ID NO: 67
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 69 - RNA, 20 nt, synthetic construct
  source 1..20: mol_type = other RNA
gcacctcttgccttacggca

SEQ ID NO: 70 - RNA, 20 nt, synthetic construct
  source 1..20: mol_type = other RNA
  misc_feature 1..4: note = modified with 2'-O-methyl phosphorothioate
gcacctcttgccttacggca

SEQ ID NO: 71 - RNA, 100 nt, synthetic construct
  source 1..100: mol_type = other RNA
ttctgaattagatccaggcggttttagagctagaaatagcaagttaaaataaggctagtccgttatcaacttgaaaaagtggcaccgagtcggtgctttt

SEQ ID NO: 72 - RNA, 100 nt, synthetic construct; identical to SEQ ID NO: 71
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 73 - RNA, 20 nt, synthetic construct
  source 1..20: mol_type = other RNA
ttctgaattagatccaggcg

SEQ ID NO: 74 - RNA, 20 nt, synthetic construct
  source 1..20: mol_type = other RNA
  misc_feature 1..4: note = modified with 2'-O-methyl phosphorothioate
ttctgaattagatccaggcg

SEQ ID NO: 75 - RNA, 100 nt, synthetic construct
  source 1..100: mol_type = other RNA
tagtcccaaacacatcccgagttttagagctagaaatagcaagttaaaataaggctagtccgttatcaacttgaaaaagtggcaccgagtcggtgctttt

SEQ ID NO: 76 - RNA, 100 nt, synthetic construct; identical to SEQ ID NO: 75
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 77 - RNA, 20 nt, synthetic construct
  source 1..20: mol_type = other RNA
tagtcccaaacacatcccga

SEQ ID NO: 78 - RNA, 20 nt, synthetic construct
  source 1..20: mol_type = other RNA
  misc_feature 1..4: note = modified with 2'-O-methyl phosphorothioate
tagtcccaaacacatcccga

SEQ ID NO: 79 - RNA, 100 nt, synthetic construct
  source 1..100: mol_type = other RNA
ttagtcccaaacacatcccggttttagagctagaaatagcaagttaaaataaggctagtccgttatcaacttgaaaaagtggcaccgagtcggtgctttt

SEQ ID NO: 80 - RNA, 100 nt, synthetic construct; identical to SEQ ID NO: 79
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 81 - RNA, 20 nt, synthetic construct
  source 1..20: mol_type = other RNA
ttagtcccaaacacatcccg

SEQ ID NO: 82 - RNA, 20 nt, synthetic construct
  source 1..20: mol_type = other RNA
  misc_feature 1..4: note = modified with 2'-O-methyl phosphorothioate
ttagtcccaaacacatcccg

SEQ ID NO: 83 - DNA, 23 bp, synthetic construct
  source 1..23: mol_type = other DNA
tacccaaaattcgaccttttcgg

SEQ ID NO: 84 - DNA, 20 bp, synthetic construct
  source 1..20: mol_type = other DNA
tacccaaaattcgacctttt

SEQ ID NO: 85 - DNA, 23 bp, synthetic construct
  source 1..23: mol_type = other DNA
ccccgaaaaggtcgaattttggg

SEQ ID NO: 86 - DNA, 20 bp, synthetic construct
  source 1..20: mol_type = other DNA
ccccgaaaaggtcgaatttt

SEQ ID NO: 87 - DNA, 23 bp, synthetic construct
  source 1..23: mol_type = other DNA
gggtattattgatgctattcagg

SEQ ID NO: 88 - DNA, 20 bp, synthetic construct
  source 1..20: mol_type = other DNA
gggtattattgatgctattc

SEQ ID NO: 89 - DNA, 23 bp, synthetic construct
  source 1..23: mol_type = other DNA
gatctgcggcagcttgcttaggg

SEQ ID NO: 90 - DNA, 20 bp, synthetic construct
  source 1..20: mol_type = other DNA
gatctgcggcagcttgctta

SEQ ID NO: 91 - DNA, 23 bp, synthetic construct
  source 1..23: mol_type = other DNA
agcaagctgccgcagatcgcagg

SEQ ID NO: 92 - DNA, 20 bp, synthetic construct
  source 1..20: mol_type = other DNA
agcaagctgccgcagatcgc

SEQ ID NO: 93 - DNA, 23 bp, synthetic construct
  source 1..23: mol_type = other DNA
tgccgcagatcgcaggaccgtgg

SEQ ID NO: 94 - DNA, 20 bp, synthetic construct
  source 1..20: mol_type = other DNA
tgccgcagatcgcaggaccg

SEQ ID NO: 95 - DNA, 23 bp, synthetic construct
  source 1..23: mol_type = other DNA
ccaatcgagtgcaacttaaccgg

SEQ ID NO: 96 - DNA, 20 bp, synthetic construct
  source 1..20: mol_type = other DNA
ccaatcgagtgcaacttaac

SEQ ID NO: 97 - DNA, 23 bp, synthetic construct
  source 1..23: mol_type = other DNA
ccggttaagttgcactcgattgg

SEQ ID NO: 98 - DNA, 20 bp, synthetic construct
  source 1..20: mol_type = other DNA
ccggttaagttgcactcgat

SEQ ID NO: 99 - DNA, 23 bp, synthetic construct
  source 1..23: mol_type = other DNA
cggttaagttgcactcgattggg

SEQ ID NO: 100 - DNA, 20 bp, synthetic construct
  source 1..20: mol_type = other DNA
cggttaagttgcactcgatt

SEQ ID NO: 101 - DNA, 23 bp, synthetic construct
  source 1..23: mol_type = other DNA
gttgcactcgattgggacagtgg

SEQ ID NO: 102 - DNA, 20 bp, synthetic construct
  source 1..20: mol_type = other DNA
gttgcactcgattgggacag

SEQ ID NO: 103 - DNA, 23 bp, synthetic construct
  source 1..23: mol_type = other DNA
ttgcactcgattgggacagtggg

SEQ ID NO: 104 - DNA, 20 bp, synthetic construct
  source 1..20: mol_type = other DNA
ttgcactcgattgggacagt

SEQ ID NO: 105 - DNA, 23 bp, synthetic construct
  source 1..23: mol_type = other DNA
gcacctcttgccttacggcatgg

SEQ ID NO: 106 - DNA, 20 bp, synthetic construct
  source 1..20: mol_type = other DNA
gcacctcttgccttacggca

SEQ ID NO: 107 - DNA, 23 bp, synthetic construct
  source 1..23: mol_type = other DNA
ttctgaattagatccaggcgagg

SEQ ID NO: 108 - DNA, 20 bp, synthetic construct
  source 1..20: mol_type = other DNA
ttctgaattagatccaggcg

SEQ ID NO: 109 - DNA, 23 bp, synthetic construct
  source 1..23: mol_type = other DNA
tagtcccaaacacatcccgaggg

SEQ ID NO: 110 - DNA, 20 bp, synthetic construct
  source 1..20: mol_type = other DNA
tagtcccaaacacatcccga

SEQ ID NO: 111 - DNA, 23 bp, synthetic construct
  source 1..23: mol_type = other DNA
ttagtcccaaacacatcccgagg

SEQ ID NO: 112 - DNA, 20 bp, synthetic construct
  source 1..20: mol_type = other DNA
ttagtcccaaacacatcccg

SEQ ID NO: 113 - DNA, 21 bp, synthetic construct
  source 1..21: mol_type = other DNA
gatcgcaggaaccgtggagaa

SEQ ID NO: 114 - DNA, 13 bp, synthetic construct
  source 1..13: mol_type = other DNA
gatcgcaggagaa

SEQ ID NO: 115 - DNA, 19 bp, synthetic construct
  source 1..19: mol_type = other DNA
gatcgcaggacgtggagaa

SEQ ID NO: 116 - DNA, 12 bp, synthetic construct
  source 1..12: mol_type = other DNA
gatcgtggagaa

SEQ ID NO: 117 - DNA, 10 bp, synthetic construct
  source 1..10: mol_type = other DNA
gatcgcagga

SEQ ID NO: 118 - DNA, 19 bp, synthetic construct
  source 1..19: mol_type = other DNA
gatcgcaggccgtggagaa

SEQ ID NO: 119 - DNA, 15 bp, synthetic construct
  source 1..15: mol_type = other DNA
gatcgccgtggagaa

SEQ ID NO: 120 - DNA, 18 bp, synthetic construct
  source 1..18: mol_type = other DNA
gatcgcagccgtggagaa

SEQ ID NO: 121 - DNA, 10 bp, synthetic construct
  source 1..10: mol_type = other DNA
ccgtggagaa

SEQ ID NO: 122 - DNA, 18 bp, synthetic construct
  source 1..18: mol_type = other DNA
gatcgcaggagtggagaa

SEQ ID NO: 123 - DNA, 11 bp, synthetic construct
  source 1..11: mol_type = other DNA
gatcgcaggaa

SEQ ID NO: 124 - DNA, 13 bp, synthetic construct
  source 1..13: mol_type = other DNA
gatccgtggagaa

SEQ ID NO: 125 - DNA, 11 bp, synthetic construct
  source 1..11: mol_type = other DNA
gatcgcaggac

SEQ ID NO: 126 - DNA, 15 bp, synthetic construct
  source 1..15: mol_type = other DNA
gatcgcagtggagaa

SEQ ID NO: 127 - DNA, 12 bp, synthetic construct
  source 1..12: mol_type = other DNA
gaccgtggagaa

SEQ ID NO: 128 - DNA, 11 bp, synthetic construct
  source 1..11: mol_type = other DNA
gccgtggagaa

SEQ ID NO: 129 - DNA, 13 bp, synthetic construct
  source 1..13: mol_type = other DNA
tgccgcaggaccg

SEQ ID NO: 130 - DNA, 21 bp, synthetic construct
  source 1..21: mol_type = other DNA
tgccgcagattcgcaggaccg

SEQ ID NO: 131 - DNA, 19 bp, synthetic construct
  source 1..19: mol_type = other DNA
tgccgcagacgcaggaccg

SEQ ID NO: 132 - DNA, 10 bp, synthetic construct
  source 1..10: mol_type = other DNA
cgcaggaccg

SEQ ID NO: 133 - DNA, 18 bp, synthetic construct
  source 1..18: mol_type = other DNA
tgccgcagcgcaggaccg

SEQ ID NO: 134 - DNA, 12 bp, synthetic construct
  source 1..12: mol_type = other DNA
tgccgcagaccg

SEQ ID NO: 135 - DNA, 17 bp, synthetic construct
  source 1..17: mol_type = other DNA
tgccgcacgcaggaccg

SEQ ID NO: 136 - DNA, 19 bp, synthetic construct
  source 1..19: mol_type = other DNA
tgccgcagatgcaggaccg

SEQ ID NO: 137 - DNA, 16 bp, synthetic construct
  source 1..16: mol_type = other DNA
tgccgccgcaggaccg

SEQ ID NO: 138 - DNA, 10 bp, synthetic construct
  source 1..10: mol_type = other DNA
tgcaggaccg

SEQ ID NO: 139 - DNA, 12 bp, synthetic construct
  source 1..12: mol_type = other DNA
tgcgcaggaccg

SEQ ID NO: 140 - DNA, 15 bp, synthetic construct
  source 1..15: mol_type = other DNA
tgccgcagatgaccg

SEQ ID NO: 141 - DNA, 21 bp, synthetic construct
  source 1..21: mol_type = other DNA
attgatgctatttcaggatgc

SEQ ID NO: 142 - DNA, 21 bp, synthetic construct
  source 1..21: mol_type = other DNA
attgatgctaattcaggatgc

SEQ ID NO: 143 - DNA, 19 bp, synthetic construct
  source 1..19: mol_type = other DNA
attgatgctttcaggatgc

SEQ ID NO: 144 - DNA, 19 bp, synthetic construct
  source 1..19: mol_type = other DNA
attgatgctatcaggatgc

SEQ ID NO: 145 - DNA, 18 bp, synthetic construct
  source 1..18: mol_type = other DNA
attgatgcttcaggatgc

SEQ ID NO: 146 - DNA, 15 bp, synthetic construct
  source 1..15: mol_type = other DNA
attgatgcaggatgc

SEQ ID NO: 147 - DNA, 22 bp, synthetic construct
  source 1..22: mol_type = other DNA
attgatgctatattcaggatgc

SEQ ID NO: 148 - DNA, 18 bp, synthetic construct
  source 1..18: mol_type = other DNA
attgatgctacaggatgc

SEQ ID NO: 149 - DNA, 11 bp, synthetic construct
  source 1..11: mol_type = other DNA
attcaggatgc

SEQ ID NO: 150 - DNA, 15 bp, synthetic construct
  source 1..15: mol_type = other DNA
attgattcaggatgc

SEQ ID NO: 151 - DNA, 15 bp, synthetic construct
  source 1..15: mol_type = other DNA
attgatgctagatgc

SEQ ID NO: 152 - DNA, 10 bp, synthetic construct
  source 1..10: mol_type = other DNA
ttcaggatgc

SEQ ID NO: 153 - DNA, 21 bp, synthetic construct
  source 1..21: mol_type = other DNA
attgatgctagttcaggatgc

SEQ ID NO: 154 - DNA, 16 bp, synthetic construct
  source 1..16: mol_type = other DNA
attgatgctaggatgc

SEQ ID NO: 155 - DNA, 22 bp, synthetic construct
  source 1..22: mol_type = other DNA
attgatgctattttcaggatgc

SEQ ID NO: 156 - DNA, 13 bp, synthetic construct
  source 1..13: mol_type = other DNA
attgatgctatgc

SEQ ID NO: 157 - DNA, 10 bp, synthetic construct
  source 1..10: mol_type = other DNA
attgatgcta

SEQ ID NO: 158 - DNA, 12 bp, synthetic construct
  source 1..12: mol_type = other DNA
atttcaggatgc

SEQ ID NO: 159 - DNA, 21 bp, synthetic construct
  source 1..21: mol_type = other DNA
ggcagcttgccttagggggtc

SEQ ID NO: 160 - DNA, 18 bp, synthetic construct
  source 1..18: mol_type = other DNA
ggcagcttgcagggggtc

SEQ ID NO: 161 - DNA, 21 bp, synthetic construct
  source 1..21: mol_type = other DNA
ggcagcttgctttagggggtc

SEQ ID NO: 162 - DNA, 19 bp, synthetic construct
  source 1..19: mol_type = other DNA
ggcagcttgctagggggtc

SEQ ID NO: 163 - DNA, 22 bp, synthetic construct
  source 1..22: mol_type = other DNA
ggcagcttgcttttagggggtc

SEQ ID NO: 164 - DNA, 21 bp, synthetic construct
  source 1..21: mol_type = other DNA
ggcagcttgcattagggggtc

SEQ ID NO: 165 - DNA, 16 bp, synthetic construct
  source 1..16: mol_type = other DNA
ggcagcttagggggtc

SEQ ID NO: 166 - DNA, 19 bp, synthetic construct
  source 1..19: mol_type = other DNA
ggcagcttgttagggggtc

SEQ ID NO: 167 - DNA, 21 bp, synthetic construct
  source 1..21: mol_type = other DNA
ggcagcttgcgttagggggtc

SEQ ID NO: 168 - DNA, 16 bp, synthetic construct
  source 1..16: mol_type = other DNA
ggcagcttgcggggtc

SEQ ID NO: 169 - DNA, 18 bp, synthetic construct
  source 1..18: mol_type = other DNA
ggcagcttttagggggtc

SEQ ID NO: 170 - DNA, 17 bp, synthetic construct
  source 1..17: mol_type = other DNA
ggcagcttgcgggggtc

SEQ ID NO: 171 - DNA, 11 bp, synthetic construct
  source 1..11: mol_type = other DNA
ggcagggggtc

SEQ ID NO: 172 - DNA, 14 bp, synthetic construct
  source 1..14: mol_type = other DNA
ggcagcttgcggtc

SEQ ID NO: 173 - DNA, 15 bp, synthetic construct
  source 1..15: mol_type = other DNA
agtgcaaccggaaaa

SEQ ID NO: 174 - DNA, 21 bp, synthetic construct
  source 1..21: mol_type = other DNA
agtgcaactttaaccggaaaa

SEQ ID NO: 175 - DNA, 19 bp, synthetic construct
  source 1..19: mol_type = other DNA
agtgcaacttaccggaaaa

SEQ ID NO: 176 - DNA, 21 bp, synthetic construct
  source 1..21: mol_type = other DNA
agtgcaacttaaaccggaaaa

SEQ ID NO: 177 - DNA, 19 bp, synthetic construct
  source 1..19: mol_type = other DNA
agtgcaactaaccggaaaa

SEQ ID NO: 178 - DNA, 10 bp, synthetic construct
  source 1..10: mol_type = other DNA
aaccggaaaa

SEQ ID NO: 179 - DNA, 14 bp, synthetic construct
  source 1..14: mol_type = other DNA
agtgaaccggaaaa

SEQ ID NO: 180 - DNA, 18 bp, synthetic construct
  source 1..18: mol_type = other DNA
agtgcaacaaccggaaaa

SEQ ID NO: 181 - DNA, 17 bp, synthetic construct
  source 1..17: mol_type = other DNA
agtgcaaaaccggaaaa

SEQ ID NO: 182 - DNA, 12 bp, synthetic construct
  source 1..12: mol_type = other DNA
cgattgggccat

SEQ ID NO: 183 - DNA, 18 bp, synthetic construct
  source 1..18: mol_type = other DNA
cgattgggagtgggccat

SEQ ID NO: 184 - DNA, 12 bp, synthetic construct
  source 1..12: mol_type = other DNA
cgagtgggccat

SEQ ID NO: 185 - DNA, 21 bp, synthetic construct
  source 1..21: mol_type = other DNA
cgattgggaccagtgggccat

SEQ ID NO: 186 - DNA, 21 bp, synthetic construct
  source 1..21: mol_type = other DNA
cgattgggacaagtgggccat

SEQ ID NO: 187 - DNA, 19 bp, synthetic construct
  source 1..19: mol_type = other DNA
cgattgggaagtgggccat

SEQ ID NO: 188 - DNA, 17 bp, synthetic construct
  source 1..17: mol_type = other DNA
cgattggagtgggccat

SEQ ID NO: 189 - DNA, 11 bp, synthetic construct
  source 1..11: mol_type = other DNA
cgattggccat

SEQ ID NO: 190 - DNA, 16 bp, synthetic construct
  source 1..16: mol_type = other DNA
cgattgagtgggccat

SEQ ID NO: 191 - DNA, 13 bp, synthetic construct
  source 1..13: mol_type = other DNA
cgattgggaccat

SEQ ID NO: 192 - DNA, 21 bp, synthetic construct
  source 1..21: mol_type = other DNA
cgattgggactagtgggccat

SEQ ID NO: 193 - DNA, 19 bp, synthetic construct
  source 1..19: mol_type = other DNA
cgattgggacgtgggccat

SEQ ID NO: 194 - DNA, 12 bp, synthetic construct
  source 1..12: mol_type = other DNA
cgattgggacat

SEQ ID NO: 195 - DNA, 18 bp, synthetic construct
  source 1..18: mol_type = other DNA
cgattgggactgggccat

SEQ ID NO: 196 - DNA, 10 bp, synthetic construct
  source 1..10: mol_type = other DNA
cgattgggac

SEQ ID NO: 197 - DNA, 10 bp, synthetic construct
  source 1..10: mol_type = other DNA
agtgggccat

SEQ ID NO: 198 - DNA, 15 bp, synthetic construct
  source 1..15: mol_type = other DNA
cgattagtgggccat

SEQ ID NO: 199 - DNA, 21 bp, synthetic construct
  source 1..21: mol_type = other DNA
ttgccttacgggcatggcagg

SEQ ID NO: 200 - DNA, 19 bp, synthetic construct
  source 1..19: mol_type = other DNA
ttgccttacgcatggcagg

SEQ ID NO: 201 - DNA, 12 bp, synthetic construct
  source 1..12: mol_type = other DNA
ttgcatggcagg

SEQ ID NO: 202 - DNA, 18 bp, synthetic construct
  source 1..18: mol_type = other DNA
ttgccttagcatggcagg

SEQ ID NO: 203 - DNA, 22 bp, synthetic construct
  source 1..22: mol_type = other DNA
ttgccttacgcggcatggcagg

SEQ ID NO: 204 - DNA, 18 bp, synthetic construct
  source 1..18: mol_type = other DNA
ttgccttggcatggcagg

SEQ ID NO: 205 - DNA, 10 bp, synthetic construct
  source 1..10: mol_type = other DNA
ttgccttacg

SEQ ID NO: 206 - DNA, 13 bp, synthetic construct
  source 1..13: mol_type = other DNA
ttggcatggcagg

SEQ ID NO: 207 - DNA, 19 bp, synthetic construct
  source 1..19: mol_type = other DNA
ttgccttaggcatggcagg

SEQ ID NO: 208 - DNA, 16 bp, synthetic construct
  source 1..16: mol_type = other DNA
ttgcctgcatggcagg

SEQ ID NO: 209 - DNA, 10 bp, synthetic construct
  source 1..10: mol_type = other DNA
gcatggcagg

SEQ ID NO: 210 - DNA, 17 bp, synthetic construct
  source 1..17: mol_type = other DNA
ttgccttgcatggcagg

SEQ ID NO: 211 - DNA, 11 bp, synthetic construct
  source 1..11: mol_type = other DNA
ggcatggcagg

SEQ ID NO: 212 - DNA, 18 bp, synthetic construct
  source 1..18: mol_type = other DNA
ttgccttacgatggcagg

SEQ ID NO: 213 - DNA, 15 bp, synthetic construct
  source 1..15: mol_type = other DNA
ttgcggcatggcagg

SEQ ID NO: 214 - DNA, 17 bp, synthetic construct
  source 1..17: mol_type = other DNA
ttgccttacatggcagg

SEQ ID NO: 215 - DNA, 13 bp, synthetic construct
  source 1..13: mol_type = other DNA
ttgccttggcagg

SEQ ID NO: 216 - DNA, 19 bp, synthetic construct
  source 1..19: mol_type = other DNA
aaacacatccgagggcacc

SEQ ID NO: 217 - DNA, 21 bp, synthetic construct
  source 1..21: mol_type = other DNA
aaacacatccccgagggcacc

SEQ ID NO: 218 - DNA, 18 bp, synthetic construct
  source 1..18: mol_type = other DNA
aaacacatcgagggcacc

SEQ ID NO: 219 - DNA, 10 bp, synthetic construct
  source 1..10: mol_type = other DNA
aaacacatcc

SEQ ID NO: 220 - DNA, 16 bp, synthetic construct
  source 1..16: mol_type = other DNA
aaacaccgagggcacc

SEQ ID NO: 221 - DNA, 15 bp, synthetic construct
  source 1..15: mol_type = other DNA
aaacacgagggcacc

SEQ ID NO: 222 - DNA, 10 bp, synthetic construct
  source 1..10: mol_type = other DNA
cgagggcacc

SEQ ID NO: 223 - DNA, 19 bp, synthetic construct
  source 1..19: mol_type = other DNA
caaacacacccgagggcac

SEQ ID NO: 224 - DNA, 17 bp, synthetic construct
  source 1..17: mol_type = other DNA
caaacacccgagggcac

SEQ ID NO: 225 - DNA, 12 bp, synthetic construct
  source 1..12: mol_type = other DNA
tcccgagggcac

SEQ ID NO: 226 - DNA, 21 bp, synthetic construct
  source 1..21: mol_type = other DNA
caaacacatccccgagggcac

SEQ ID NO: 227 - DNA, 18 bp, synthetic construct
  source 1..18: mol_type = other DNA
caaacacaccgagggcac

SEQ ID NO: 228 - DNA, 10 bp, synthetic construct
  source 1..10: mol_type = other DNA
ccgagggcac

SEQ ID NO: 229 - DNA, 18 bp, synthetic construct
  source 1..18: mol_type = other DNA
caaacacatcgagggcac

SEQ ID NO: 230 - DNA, 16 bp, synthetic construct
  source 1..16: mol_type = other DNA
caaacaccgagggcac

SEQ ID NO: 231 - DNA, 11 bp, synthetic construct
  source 1..11: mol_type = other DNA
cccgagggcac

SEQ ID NO: 232 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 233 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 234 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 235 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 236 - DNA, 800 bp, synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 237 - DNA, 804 bp, synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 238 - DNA, 1178 bp, synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 239 - DNA, 4364 bp, synthetic construct
  source 1..4364: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 240 - DNA, 4364 bp, synthetic construct
  source 1..4364: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 241 - protein, 22 aa, synthetic construct
MLLLVTSLLLCELPHPAFLLIP

SEQ ID NO: 242 - protein, 21 aa, synthetic construct
MALPVTALLLPLALLLHAARP

SEQ ID NO: 243 - protein, 23 aa, synthetic construct
IYIWAPLAGTCGVLLLSLVITLY

SEQ ID NO: 244 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 245 - protein, 42 aa, synthetic construct
KRGRKKLLYIFKQPFMRPVQTTQEEDGCSCRFPEEEEGGCEL

SEQ ID NO: 246 - DNA, 120 bp, synthetic construct
  source 1..120: mol_type = other DNA
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 247 - protein, 40 aa, synthetic construct
SKRSRLLHSDYMNMTPRRPGPTRKHYQPYAPPRDFAAYRS

SEQ ID NO: 248 - DNA, 336 bp, synthetic construct
  source 1..336: mol_type = other DNA
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 249 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 250 - DNA, 261 bp, synthetic construct
  source 1..261: mol_type = other DNA
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 251 - DNA, 252 bp, synthetic construct
  source 1..252: mol_type = other DNA
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 252 - protein, 84 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 253 - protein, 15 aa, synthetic construct
RASKSVSTSGYSFMH

SEQ ID NO: 254 - protein, 11 aa, synthetic construct
SKSVSTSGYSF

SEQ ID NO: 255 - protein, 7 aa, synthetic construct
LASNLES

SEQ ID NO: 256 - DNA, 19 bp, synthetic construct
  source 1..19: mol_type = other DNA
caaacacatccgagggcac

SEQ ID NO: 257 - protein, 9 aa, synthetic construct
QHSREVPWT

SEQ ID NO: 258 - protein, 6 aa, synthetic construct
SREVPW

SEQ ID NO: 259 - protein, 5 aa, synthetic construct
NYGMN

SEQ ID NO: 260 - protein, 10 aa, synthetic construct
GYTFTNYGMN

SEQ ID NO: 261 - protein, 17 aa, synthetic construct
WINTYTGEPTYADAFKG

SEQ ID NO: 262 - protein, 6 aa, synthetic construct
NTYTGE

SEQ ID NO: 263 - protein, 9 aa, synthetic construct
DYGDYGMDY

SEQ ID NO: 264 - protein, 14 aa, synthetic construct
CARDYGDYGMDYWG

SEQ ID NO: 265 - protein, 508 aa, synthetic construct
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 266 - protein, 487 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 267 - DNA, 735 bp, synthetic construct
  source 1..735: mol_type = other DNA
gatatagttatgacccaatcacccgatagtcttgcggtaagcctgggggagcgagcaacaataaactgtcgggcatcaaaatccgtcagtacaagcgggtattcattcatgcactggtatcaacagaaacccggtcagccacccaagctcctgatttatcttgcgtctaatcttgagtccggcgtcccagaccggttttccggctccgggagcggcacggattttactcttactatttctagccttcaggccgaagatgtggcggtatactactgccagcattcaagggaagttccttggacgttcggtcagggcacgaaagtggaaattaaaggcggggggggatccggcgggggagggtctggaggaggtggcagtggtcaggtccaactggtgcagtccggggcagaggtaaaaaaacccggcgcgtctgttaaggtttcatgcaaggccagtggatatactttcaccaattacggaatgaactgggtgaggcaggcccctggtcaaggcctgaaatggatgggatggataaacacgtacaccggtgaacctacctatgccgatgcctttaagggtcgggttacgatgacgagagacacctccatatcaacagcctacatggagctcagcagattgaggagtgacgatacggcagtctattactgtgcaagagactacggcgattatggcatggattactggggccagggcactacagtaaccgtttccagc

SEQ ID NO: 268 - protein, 245 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGGGGSGGGGSGGGGSGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 269 - DNA, 735 bp, synthetic construct
  source 1..735: mol_type = other DNA
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 270 - protein, 245 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 271 - protein, 118 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 272 - protein, 111 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 273 - DNA, 1524 bp, synthetic construct
  source 1..1524: mol_type = other DNA
atggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 274 - protein, 508 aa, synthetic construct
MALPVTALLLPLALLLHAARPQVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 275 - protein, 487 aa, synthetic construct
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 276 - protein, 735 aa, synthetic construct
CAGGTGCAGCTGGTGCAGAGCGGAGCCGAGCTCAAGAAGCCCGGAGCCTCCGTGAAGGTGAGCTGCAAGGCCAGCGGCAACACCCTGACCAACTACGTGATCCACTGGGTGAGACAAGCCCCCGGCCAAAGGCTGGAGTGGATGGGCTACATCCTGCCCTACAACGACCTGACCAAGTACAGCCAGAAGTTCCAGGGCAGGGTGACCATCACCAGGGATAAGAGCGCCTCCACCGCCTATATGGAGCTGAGCAGCCTGAGGAGCGAGGACACCGCTGTGTACTACTGTACAAGGTGGGACTGGGACGGCTTCTTTGACCCCTGGGGCCAGGGCACAACAGTGACCGTCAGCAGCGGCGGCGGAGGCAGCGGCGGCGGCGGCAGCGGCGGAGGCGGAAGCGAAATCGTGATGACCCAGAGCCCCGCCACACTGAGCGTGAGCCCTGGCGAGAGGGCCAGCATCTCCTGCAGGGCTAGCCAAAGCCTGGTGCACAGCAACGGCAACACCCACCTGCACTGGTACCAGCAGAGACCCGGACAGGCTCCCAGGCTGCTGATCTACAGCGTGAGCAACAGGTTCTCCGAGGTGCCTGCCAGGTTTAGCGGCAGCGGAAGCGGCACCGACTTTACCCTGACCATCAGCAGCGTGGAGTCCGAGGACTTCGCCGTGTATTACTGCAGCCAGACCAGCCACATCCCTTACACCTTCGGCGGCGGCACCAAGCTGGAGATCAAA

SEQ ID NO: 277 - protein, 245 aa, synthetic construct
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 278 - protein, 118 aa, synthetic construct
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSS

SEQ ID NO: 279 - protein, 112 aa, synthetic construct
EIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 280 - protein, 16 aa, synthetic construct
RASQSLVHSNGNTHLH

SEQ ID NO: 281 - protein, 7 aa, synthetic construct
SVSNRFS

SEQ ID NO: 282 - protein, 9 aa, synthetic construct
SQTSHIPYT

SEQ ID NO: 283 - protein, 9 aa, synthetic construct
SQTSHIPYT

SEQ ID NO: 284 - protein, 5 aa, synthetic construct
NYVIH

SEQ ID NO: 285 - protein, 7 aa, synthetic construct
GNTLTNY

SEQ ID NO: 286 - protein, 17 aa, synthetic construct
YILPYNDLTKYSQKFQG

SEQ ID NO: 287 - protein, 6 aa, synthetic construct
LPYNDL

SEQ ID NO: 288 - protein, 9 aa, synthetic construct
WDWDGFFDP

SEQ ID NO: 289 - protein, 9 aa, synthetic construct
WDWDGFFDP

SEQ ID NO: 290 - DNA, 20 bp, synthetic construct
  source 1..20: mol_type = other DNA
gatcgcaggaccgtggagaa

SEQ ID NO: 291 - DNA, 20 bp, synthetic construct
  source 1..20: mol_type = other DNA
tgccgcagatcgcaggaccg

SEQ ID NO: 292 - DNA, 20 bp, synthetic construct
  source 1..20: mol_type = other DNA
attgatgctattcaggatgc

SEQ ID NO: 293 - DNA, 20 bp, synthetic construct
  source 1..20: mol_type = other DNA
ggcagcttgcttagggggtc

SEQ ID NO: 294 - DNA, 20 bp, synthetic construct
  source 1..20: mol_type = other DNA
agtgcaacttaaccggaaaa

SEQ ID NO: 295 - DNA, 20 bp, synthetic construct
  source 1..20: mol_type = other DNA
cgattgggacagtgggccat

SEQ ID NO: 296 - DNA, 20 bp, synthetic construct
  source 1..20: mol_type = other DNA
ttgccttacggcatggcagg

SEQ ID NO: 297 - DNA, 20 bp, synthetic construct
  source 1..20: mol_type = other DNA
aaacacatcccgagggcacc

SEQ ID NO: 298 - DNA, 20 bp, synthetic construct
  source 1..20: mol_type = other DNA
caaacacatcccgagggcac

SEQ ID NO: 299 - RNA, 100 nt, synthetic construct
  source 1..100: mol_type = other RNA
ggtcatcgatgggagcaacggttttagagctagaaatagcaagttaaaataaggctagtccgttatcaacttgaaaaagtggcaccgagtcggtgctttt

SEQ ID NO: 300 - RNA, 100 nt, synthetic construct; identical to SEQ ID NO: 299
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 301 - RNA, 20 nt, synthetic construct
  source 1..20: mol_type = other RNA
ggtcatcgatgggagcaacg

SEQ ID NO: 302 - RNA, 20 nt, synthetic construct
  source 1..20: mol_type = other RNA
  misc_feature 1..4: note = modified with 2'-O-methyl phosphorothioate
ggtcatcgatgggagcaacg

SEQ ID NO: 303 - RNA, 100 nt, synthetic construct
  source 1..100: mol_type = other RNA
caccaccccgcgggactagagttttagagctagaaatagcaagttaaaataaggctagtccgttatcaacttgaaaaagtggcaccgagtcggtgctttt

SEQ ID NO: 304 - RNA, 100 nt, synthetic construct; identical to SEQ ID NO: 303
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 305 - RNA, 20 nt, synthetic construct
  source 1..20: mol_type = other RNA
caccaccccgcgggactaga

SEQ ID NO: 306 - RNA, 20 nt, synthetic construct
  source 1..20: mol_type = other RNA
  misc_feature 1..4: note = modified with 2'-O-methyl phosphorothioate
caccaccccgcgggactaga

SEQ ID NO: 307 - RNA, 100 nt, synthetic construct
  source 1..100: mol_type = other RNA
ggtctggcgctcccgctcgggttttagagctagaaatagcaagttaaaataaggctagtccgttatcaacttgaaaaagtggcaccgagtcggtgctttt

SEQ ID NO: 308 - RNA, 100 nt, synthetic construct; identical to SEQ ID NO: 307
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 309 - RNA, 20 nt, synthetic construct
  source 1..20: mol_type = other RNA
ggtctggcgctcccgctcgg

SEQ ID NO: 310 - RNA, 20 nt, synthetic construct
  source 1..20: mol_type = other RNA
  misc_feature 1..4: note = modified with 2'-O-methyl phosphorothioate
ggtctggcgctcccgctcgg

SEQ ID NO: 311 - RNA, 100 nt, synthetic construct
  source 1..100: mol_type = other RNA
ttcacaccatcacgacgcgtgttttagagctagaaatagcaagttaaaataaggctagtccgttatcaacttgaaaaagtggcaccgagtcggtgctttt

SEQ ID NO: 312 - RNA, 100 nt, synthetic construct; identical to SEQ ID NO: 311
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 313 - RNA, 20 nt, synthetic construct
  source 1..20: mol_type = other RNA
ttcacaccatcacgacgcgt

SEQ ID NO: 314 - RNA, 20 nt, synthetic construct
  source 1..20: mol_type = other RNA
  misc_feature 1..4: note = modified with 2'-O-methyl phosphorothioate
ttcacaccatcacgacgcgt

SEQ ID NO: 315 - RNA, 100 nt, synthetic construct
  source 1..100: mol_type = other RNA
acaccatcacgacgcgtggggttttagagctagaaatagcaagttaaaataaggctagtccgttatcaacttgaaaaagtggcaccgagtcggtgctttt

SEQ ID NO: 316 - RNA, 100 nt, synthetic construct; identical to SEQ ID NO: 315
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 317 - RNA, 20 nt, synthetic construct
  source 1..20: mol_type = other RNA
acaccatcacgacgcgtggg

SEQ ID NO: 318 - RNA, 20 nt, synthetic construct
  source 1..20: mol_type = other RNA
  misc_feature 1..4: note = modified with 2'-O-methyl phosphorothioate
acaccatcacgacgcgtggg

SEQ ID NO: 319 - RNA, 100 nt, synthetic construct
  source 1..100: mol_type = other RNA
ctacgagtctgacgggatcggttttagagctagaaatagcaagttaaaataaggctagtccgttatcaacttgaaaaagtggcaccgagtcggtgctttt

SEQ ID NO: 320 - RNA, 100 nt, synthetic construct; identical to SEQ ID NO: 319
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 321 - RNA, 20 nt, synthetic construct
  source 1..20: mol_type = other RNA
ctacgagtctgacgggatcg

SEQ ID NO: 322 - RNA, 20 nt, synthetic construct
  source 1..20: mol_type = other RNA
  misc_feature 1..4: note = modified with 2'-O-methyl phosphorothioate
ctacgagtctgacgggatcg

SEQ ID NO: 323 - RNA, 100 nt, synthetic construct
  source 1..100: mol_type = other RNA
ttgccacccacgcgtcgtgagttttagagctagaaatagcaagttaaaataaggctagtccgttatcaacttgaaaaagtggcaccgagtcggtgctttt

SEQ ID NO: 324 - RNA, 100 nt, synthetic construct; identical to SEQ ID NO: 323
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 325 - RNA, 20 nt, synthetic construct
  source 1..20: mol_type = other RNA
ttgccacccacgcgtcgtga

SEQ ID NO: 326 - RNA, 20 nt, synthetic construct
  source 1..20: mol_type = other RNA
  misc_feature 1..4: note = modified with 2'-O-methyl phosphorothioate
ttgccacccacgcgtcgtga

SEQ ID NO: 327 - RNA, 100 nt, synthetic construct
  source 1..100: mol_type = other RNA
gttcacaccatcacgacgcggttttagagctagaaatagcaagttaaaataaggctagtccgttatcaacttgaaaaagtggcaccgagtcggtgctttt

SEQ ID NO: 328 - RNA, 100 nt, synthetic construct; identical to SEQ ID NO: 327
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 329 - RNA, 20 nt, synthetic construct
  source 1..20: mol_type = other RNA
gttcacaccatcacgacgcg

SEQ ID NO: 330 - RNA, 20 nt, synthetic construct
  source 1..20: mol_type = other RNA
  misc_feature 1..4: note = modified with 2'-O-methyl phosphorothioate
gttcacaccatcacgacgcg

SEQ ID NO: 331 - RNA, 100 nt, synthetic construct
  source 1..100: mol_type = other RNA
cacgatcccgtcagactcgtgttttagagctagaaatagcaagttaaaataaggctagtccgttatcaacttgaaaaagtggcaccgagtcggtgctttt

SEQ ID NO: 332 - RNA, 100 nt, synthetic construct; identical to SEQ ID NO: 331
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 333 - RNA, 20 nt, synthetic construct
  source 1..20: mol_type = other RNA
cacgatcccgtcagactcgt

SEQ ID NO: 334 - RNA, 20 nt, synthetic construct
  source 1..20: mol_type = other RNA
  misc_feature 1..4: note = modified with 2'-O-methyl phosphorothioate
cacgatcccgtcagactcgt

SEQ ID NO: 335 - RNA, 100 nt, synthetic construct
  source 1..100: mol_type = other RNA
acgacgcgtgggtggcaagcgttttagagctagaaatagcaagttaaaataaggctagtccgttatcaacttgaaaaagtggcaccgagtcggtgctttt

SEQ ID NO: 336 - RNA, 100 nt, synthetic construct; identical to SEQ ID NO: 335
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 337 - RNA, 20 nt, synthetic construct
  source 1..20: mol_type = other RNA
acgacgcgtgggtggcaagc

SEQ ID NO: 338 - RNA, 20 nt, synthetic construct
  source 1..20: mol_type = other RNA
  misc_feature 1..4: note = modified with 2'-O-methyl phosphorothioate
acgacgcgtgggtggcaagc

SEQ ID NO: 339 - DNA, 23 bp, synthetic construct
  source 1..23: mol_type = other DNA
ggtcatcgatgggagcaacgtgg

SEQ ID NO: 340 - DNA, 20 bp, synthetic construct
  source 1..20: mol_type = other DNA
ggtcatcgatgggagcaacg

SEQ ID NO: 341 - DNA, 23 bp, synthetic construct
  source 1..23: mol_type = other DNA
caccaccccgcgggactagaggg

SEQ ID NO: 342 - DNA, 20 bp, synthetic construct
  source 1..20: mol_type = other DNA
caccaccccgcgggactaga

SEQ ID NO: 343 - DNA, 23 bp, synthetic construct
  source 1..23: mol_type = other DNA
ggtctggcgctcccgctcggtgg

SEQ ID NO: 344 - DNA, 20 bp, synthetic construct
  source 1..20: mol_type = other DNA
ggtctggcgctcccgctcgg

SEQ ID NO: 345 - DNA, 23 bp, synthetic construct
  source 1..23: mol_type = other DNA
ttcacaccatcacgacgcgtggg

SEQ ID NO: 346 - DNA, 20 bp, synthetic construct
  source 1..20: mol_type = other DNA
ttcacaccatcacgacgcgt

SEQ ID NO: 347 - DNA, 23 bp, synthetic construct
  source 1..23: mol_type = other DNA
acaccatcacgacgcgtgggtgg

SEQ ID NO: 348 - DNA, 20 bp, synthetic construct
  source 1..20: mol_type = other DNA
acaccatcacgacgcgtggg

SEQ ID NO: 349 - DNA, 23 bp, synthetic construct
  source 1..23: mol_type = other DNA
ctacgagtctgacgggatcgtgg

SEQ ID NO: 350 - DNA, 20 bp, synthetic construct
  source 1..20: mol_type = other DNA
ctacgagtctgacgggatcg

SEQ ID NO: 351 - DNA, 23 bp, synthetic construct
  source 1..23: mol_type = other DNA
ttgccacccacgcgtcgtgatgg

SEQ ID NO: 352 - DNA, 20 bp, synthetic construct
  source 1..20: mol_type = other DNA
ttgccacccacgcgtcgtga

SEQ ID NO: 353 - DNA, 23 bp, synthetic construct
  source 1..23: mol_type = other DNA
gttcacaccatcacgacgcgtgg

SEQ ID NO: 354 - DNA, 20 bp, synthetic construct
  source 1..20: mol_type = other DNA
gttcacaccatcacgacgcg

SEQ ID NO: 355 - DNA, 23 bp, synthetic construct
  source 1..23: mol_type = other DNA
cacgatcccgtcagactcgtagg

SEQ ID NO: 356 - DNA, 20 bp, synthetic construct
  source 1..20: mol_type = other DNA
cacgatcccgtcagactcgt

SEQ ID NO: 357 - DNA, 23 bp, synthetic construct
  source 1..23: mol_type = other DNA
acgacgcgtgggtggcaagcggg

SEQ ID NO: 358 - DNA, 20 bp, synthetic construct
  source 1..20: mol_type = other DNA
acgacgcgtgggtggcaagc

SEQ ID NO: 359 - RNA, 100 nt, synthetic construct
  source 1..100: mol_type = other RNA
ccgacttctgaacgtgcggtgttttagagctagaaatagcaagttaaaataaggctagtccgttatcaacttgaaaaagtggcaccgagtcggtgctttt

SEQ ID NO: 360 - RNA, 100 nt, synthetic construct; identical to SEQ ID NO: 359
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 361 - RNA, 20 nt, synthetic construct
  source 1..20: mol_type = other RNA
ccgacttctgaacgtgcggt

SEQ ID NO: 362 - RNA, 20 nt, synthetic construct
  source 1..20: mol_type = other RNA
  misc_feature 1..4: note = modified with 2'-O-methyl phosphorothioate
ccgacttctgaacgtgcggt

SEQ ID NO: 363 - RNA, 100 nt, synthetic construct
  source 1..100: mol_type = other RNA
tgctggcgatacgcgtccacgttttagagctagaaatagcaagttaaaataaggctagtccgttatcaacttgaaaaagtggcaccgagtcggtgctttt

SEQ ID NO: 364 - RNA, 100 nt, synthetic construct; identical to SEQ ID NO: 363
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 365 - RNA, 20 nt, synthetic construct
  source 1..20: mol_type = other RNA
tgctggcgatacgcgtccac

SEQ ID NO: 366 - RNA, 20 nt, synthetic construct
  source 1..20: mol_type = other RNA
  misc_feature 1..4: note = modified with 2'-O-methyl phosphorothioate
tgctggcgatacgcgtccac

SEQ ID NO: 367 - RNA, 100 nt, synthetic construct
  source 1..100: mol_type = other RNA
tcggtctatgacgagcagcggttttagagctagaaatagcaagttaaaataaggctagtccgttatcaacttgaaaaagtggcaccgagtcggtgctttt

SEQ ID NO: 368 - RNA, 100 nt, synthetic construct; identical to SEQ ID NO: 367
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 369 - RNA, 20 nt, synthetic construct
  source 1..20: mol_type = other RNA
tcggtctatgacgagcagcg

SEQ ID NO: 370 - RNA, 20 nt, synthetic construct
  source 1..20: mol_type = other RNA
  misc_feature 1..4: note = modified with 2'-O-methyl phosphorothioate
tcggtctatgacgagcagcg

SEQ ID NO: 371 - RNA, 100 nt, synthetic construct
  source 1..100: mol_type = other RNA
atgggcagtcctattacagcgttttagagctagaaatagcaagttaaaataaggctagtccgttatcaacttgaaaaagtggcaccgagtcggtgctttt

SEQ ID NO: 372 - RNA, 100 nt, synthetic construct; identical to SEQ ID NO: 371
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 373 - RNA, 20 nt, synthetic construct
  source 1..20: mol_type = other RNA
atgggcagtcctattacagc

SEQ ID NO: 374 - RNA, 20 nt, synthetic construct
  source 1..20: mol_type = other RNA
  misc_feature 1..4: note = modified with 2'-O-methyl phosphorothioate
atgggcagtcctattacagc

SEQ ID NO: 375 - RNA, 100 nt, synthetic construct
  source 1..100: mol_type = other RNA
attgttcacttgttagccccgttttagagctagaaatagcaagttaaaataaggctagtccgttatcaacttgaaaaagtggcaccgagtcggtgctttt

SEQ ID NO: 376 - RNA, 100 nt, synthetic construct; identical to SEQ ID NO: 375
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 377 - RNA, 20 nt, synthetic construct
  source 1..20: mol_type = other RNA
attgttcacttgttagcccc

SEQ ID NO: 378 - RNA, 20 nt, synthetic construct
  source 1..20: mol_type = other RNA
  misc_feature 1..4: note = modified with 2'-O-methyl phosphorothioate
attgttcacttgttagcccc

SEQ ID NO: 379 - RNA, 100 nt, synthetic construct
  source 1..100: mol_type = other RNA
gctgaagaactgcctctatagttttagagctagaaatagcaagttaaaataaggctagtccgttatcaacttgaaaaagtggcaccgagtcggtgctttt

SEQ ID NO: 380 - RNA, 100 nt, synthetic construct; identical to SEQ ID NO: 379
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 381 - RNA, 20 nt, synthetic construct
  source 1..20: mol_type = other RNA
gctgaagaactgcctctata

SEQ ID NO: 382 - RNA, 20 nt, synthetic construct
  source 1..20: mol_type = other RNA
  misc_feature 1..4: note = modified with 2'-O-methyl phosphorothioate
gctgaagaactgcctctata

SEQ ID NO: 383 - RNA, 100 nt, synthetic construct
  source 1..100: mol_type = other RNA
gcaggatttctggttgtcacgttttagagctagaaatagcaagttaaaataaggctagtccgttatcaacttgaaaaagtggcaccgagtcggtgctttt

SEQ ID NO: 384 - RNA, 100 nt, synthetic construct; identical to SEQ ID NO: 383
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 385 - RNA, 20 nt, synthetic construct
  source 1..20: mol_type = other RNA
gcaggatttctggttgtcac

SEQ ID NO: 386 - RNA, 20 nt, synthetic construct
  source 1..20: mol_type = other RNA
  misc_feature 1..4: note = modified with 2'-O-methyl phosphorothioate
gcaggatttctggttgtcac

SEQ ID NO: 387 - RNA, 100 nt, synthetic construct
  source 1..100: mol_type = other RNA
ctccatctgtgagaagccacgttttagagctagaaatagcaagttaaaataaggctagtccgttatcaacttgaaaaagtggcaccgagtcggtgctttt

SEQ ID NO: 388 - RNA, 100 nt, synthetic construct; identical to SEQ ID NO: 387
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 389 - RNA, 20 nt, synthetic construct
  source 1..20: mol_type = other RNA
ctccatctgtgagaagccac

SEQ ID NO: 390 - RNA, 20 nt, synthetic construct
  source 1..20: mol_type = other RNA
  misc_feature 1..4: note = modified with 2'-O-methyl phosphorothioate
ctccatctgtgagaagccac

SEQ ID NO: 391 - RNA, 100 nt, synthetic construct
  source 1..100: mol_type = other RNA
cccctaccatgactttattcgttttagagctagaaatagcaagttaaaataaggctagtccgttatcaacttgaaaaagtggcaccgagtcggtgctttt

SEQ ID NO: 392 - RNA, 100 nt, synthetic construct; identical to SEQ ID NO: 391
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 393 - RNA, 20 nt, synthetic construct
  source 1..20: mol_type = other RNA
cccctaccatgactttattc

SEQ ID NO: 394 - RNA, 20 nt, synthetic construct
  source 1..20: mol_type = other RNA
  misc_feature 1..4: note = modified with 2'-O-methyl phosphorothioate
cccctaccatgactttattc

SEQ ID NO: 395 - DNA, 23 bp, synthetic construct
  source 1..23: mol_type = other DNA
ccgacttctgaacgtgcggtggg

SEQ ID NO: 396 - DNA, 20 bp, synthetic construct
  source 1..20: mol_type = other DNA
ccgacttctgaacgtgcggt

SEQ ID NO: 397 - DNA, 23 bp, synthetic construct
  source 1..23: mol_type = other DNA
tgctggcgatacgcgtccacagg

SEQ ID NO: 398 - DNA, 20 bp, synthetic construct
  source 1..20: mol_type = other DNA
tgctggcgatacgcgtccac

SEQ ID NO: 399 - DNA, 23 bp, synthetic construct
  source 1..23: mol_type = other DNA
tcggtctatgacgagcagcgggg

SEQ ID NO: 400 - DNA, 20 bp, synthetic construct
  source 1..20: mol_type = other DNA
tcggtctatgacgagcagcg

SEQ ID NO: 401 - DNA, 23 bp, synthetic construct
  source 1..23: mol_type = other DNA
atgggcagtcctattacagctgg

SEQ ID NO: 402 - DNA, 20 bp, synthetic construct
  source 1..20: mol_type = other DNA
atgggcagtcctattacagc

SEQ ID NO: 403 - DNA, 23 bp, synthetic construct
  source 1..23: mol_type = other DNA
attgttcacttgttagccccagg

SEQ ID NO: 404 - DNA, 20 bp, synthetic construct
  source 1..20: mol_type = other DNA
attgttcacttgttagcccc

SEQ ID NO: 405 - DNA, 23 bp, synthetic construct
  source 1..23: mol_type = other DNA
gctgaagaactgcctctatatgg

SEQ ID NO: 406 - DNA, 20 bp, synthetic construct
  source 1..20: mol_type = other DNA
gctgaagaactgcctctata

SEQ ID NO: 407 - DNA, 23 bp, synthetic construct
  source 1..23: mol_type = other DNA
gcaggatttctggttgtcacagg

SEQ ID NO: 408 - DNA, 20 bp, synthetic construct
  source 1..20: mol_type = other DNA
gcaggatttctggttgtcac

SEQ ID NO: 409 - DNA, 23 bp, synthetic construct
  source 1..23: mol_type = other DNA
ctccatctgtgagaagccacagg

SEQ ID NO: 410 - DNA, 20 bp, synthetic construct
  source 1..20: mol_type = other DNA
ctccatctgtgagaagccac

SEQ ID NO: 411 - DNA, 23 bp, synthetic construct
  source 1..23: mol_type = other DNA
cccctaccatgactttattctgg

SEQ ID NO: 412 - DNA, 20 bp, synthetic construct
  source 1..20: mol_type = other DNA
cccctaccatgactttattc

(19) United States
(12) Patent Application Publication
(10) Pub. No.: US 2023/0346836 A1
(43) Pub. Date: Nov. 2, 2023

(54) GENETICALLY ENGINEERED T CELLS WITH DISRUPTED CASITAS B-LINEAGE LYMPHOMA PROTO-ONCOGENE-B (CBLB) AND USES THEREOF

(71) Applicant: CRISPR Therapeutics AG, Zug (CH)

(72) Inventors: Changan GUO, Cambridge, MA (US); Hanspeter WALDNER, Cambridge, MA (US); Jonathan Alexander TERRETT, Cambridge, MA (US)

(21) Appl. No.: 18/069,693

(22) Filed: Dec. 21, 2022

Related U.S. Application Data
(60) Provisional application No. 63/292,715, filed on Dec. 22, 2021.

(51) Int. Cl. A61K 35/17; C07K 14/705; C12N 9/22; C12N 15/90; C07K 14/725

(52) U.S. Cl. CPC A61K 35/17; C07K 14/7051; C07K 14/70575; C12N 9/22; C12N 15/907; C12N 2310/20; C12N 2510/00

(57) ABSTRACT

A population of genetically engineered T cells, comprising a disrupted cbl-b gene. Such genetically engineered T cells may comprise further genetic modifications, for example, a disrupted CD70 gene. The population of genetically engineered T cells exhibit one or more of (a) improved cell growth activity; (b) enhanced persistence; (c) reduced T cell exhaustion, and (d) enhanced cytotoxicity activity, as compared to non-engineered T cell counterparts.

### CROSS REFERENCE TO RELATED APPLICATIONS

This application claims the benefit of the filing dates of U.S. Provisional Application No. 63/292,715, filed on December 22, 2021, the entire contents of which are incorporated by reference herein.

### REFERENCE TO SEQUENCE LISTING

The instant application contains a Sequence Listing which has been filed electronically in XML format and is hereby incorporated by reference in its entirety. Said XML copy, created on December 19, 2022, is named 095136-0733-053WO1_SEQ.XML and is 152,364 bytes in size.

### BACKGROUND OF THE INVENTION

Chimeric antigen receptor (CAR) T-cell therapy uses genetically modified T cells to target and kill cancer cells more specifically and efficiently. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

T cells having improved cytotoxicity and persistence in culture are desired in CAR T therapy. Such T cells live longer in both in vitro and in vivo, thereby conferring benefits in CAR T cell manufacturing and clinical applications. However, it remains challenging to enhance cytotoxicity and improve persistence of T cells in culture.

### SUMMARY OF THE INVENTION

The present disclosure is based, at least in part, on the development of genetically edited T cells carrying a disrupted Casitas B-Lineage Lymphoma Proto-Oncogene-B gene (cbl-b gene, CBLB Knockout T cells), and optionally additional gene edits, for example, a knock-in of a transgene encoding a chimeric antigen receptor (CAR), a disrupted cluster of differentiation 70 (CD70) gene, a disrupted T cell receptor alpha chain constant region (TRAC) gene, a disrupted beta-2-microglobulin (β2M) gene, a disrupted Regnase-1 (Reg1) gene, a disrupted Transforming Growth Factor Beta Receptor II (TGFBRII) gene, or a combination thereof, and effective methods of producing such genetically edited T cells via, e.g., CRISPR/Cas-mediated gene editing using guide RNAs, for example, those targeting specific sites within the cbl-b gene with high on-target editing efficiency and low off-target editing efficiency. Such genetically engineered T cells exhibits the following advantageous features including, but not limited to: (a) enhanced cell killing capacity; (b) enhanced persistence; (c) improved cell growth activity; and (c) reduced T cell exhaustion. CAR-T cells with a disrupted cblb gene as disclosed herein also showed enhanced anti-tumor activity and prolonged survival rates as observed in animal models.

Accordingly, the present disclosure provides, in some aspects, a population of genetically engineered T cells, comprising: a disrupted cbl-b gene. The population of genetically engineered T cells disclosed herein, as compared to non-engineered T cell counterparts, has one or more of the following features: (a) enhanced cell killing capacity; (b) enhanced persistence; (c) improved cell growth activity; and (c) reduced T cell exhaustion.

In some embodiments, the disrupted cbl-b gene is genetically edited in exon 2, exon 7, exon 9, exon 11, or exon 12. In one example, the disrupted cbl-b gene is genetically edited in exon 2. In some embodiments, the disrupted cbl-b gene may be genetically edited by a CRISPR/Cas-mediated gene editing system. Such a CRISPR/Cas-mediated gene editing system comprises a guide RNA (gRNA) targeting a site in the cbl-b gene that comprises a nucleotide sequence of SEQ ID NO: 84, 86, 88, 90, 92, 94, 96, 98, 100, 102, 104, 106, 108, 110, or 112. In one example, the gRNA may target a site within exon 2 of the cbl-b gene, for example, targeting the site of SEQ ID NO: 92. In some examples, the gRNA may target a site within exon 7, for example, target the site of SEQ ID NO:96 or SEQ ID NO: 104. In some examples, the gRNA may target a site within exon 9, for example, target the site of SEQ ID NO: 106. In some instances, the gRNA comprises a spacer having a nucleotide sequence of SEQ ID NOs: 25, 29, 33, 37, 41, 45, 49, 53, 57, 61, 65, 69, 73, 77, or 81. In some examples, the gRNA may comprise a spacer having a nucleotide sequence of SEQ ID NO: 41, 33, 49, 65, or 69.

Any of the gRNAs may further comprises a scaffold sequence. In specific examples, the gRNA may comprise a nucleotide sequence of SEQ ID NO: 23, 27, 31, 35, 39, 43, 47, 51, 55, 59, 63, 67, 71, 75, or 79. In specific examples, the gRNA may comprise the nucleotide sequence of SEQ ID NO: 39 or SEQ ID NO: 40. In other specific examples, the gRNA may comprise the nucleotide sequence of SEQ ID NO:31 or 32. In yet other specific examples, the gRNA may comprise the nucleotide sequence of SEQ ID NO: 47 or 48. In other examples, the gRNA may comprise the nucleotide sequence of SEQ ID NO: 63 or 64. Alternatively, the gRNA may comprise the nucleotide sequence of SEQ ID NO:67 or 68.

Any of the genetically engineered T cells disclosed herein may further comprise a disrupted T cell receptor alpha chain constant region (TRAC) gene, a disrupted beta-2-microglobulin (β2M) gene, a disrupted CD70 gene, or a combination thereof. Alternatively, or in addition, the genetically engineered T cells may be engineered to express a chimeric antigen receptor (CAR).

In some embodiments, the genetically engineered T cells may further comprise a disrupted TRAC gene, which may have a deleted fragment comprising AGAGCAACAGTGCTGTGGCC (SEQ ID NO: 14). In some instances, the genetically engineered T cells comprise a nucleic acid encoding the CAR, which may be inserted in the genome of the T cells. In some examples, the genetically engineered T cells may comprise the disrupted TRAC gene, which comprises the nucleic acid encoding the CAR. In one specific example, the nucleic acid encoding the CAR replaces the deleted fragment in the disrupted TRAC gene.

In some embodiments, any of the genetically engineered T cells disclosed herein nay further comprise a disrupted Regnase-1 (Reg1) gene, a disrupted Transforming Growth Factor Beta Receptor II (TGFBRII) gene, or a combination thereof. In some instances, the genetically engineered T cells may comprise both a disrupted Reg1 gene and a disrupted TGFBRII gene.

In some examples, the genetically engineered T cells disclosed herein express a CAR (e.g., a CAR targeting a tumor antigen), a disrupted TRAC gene, a disrupted Reg1 gene, a disrupted TGFBRII gene, and optionally a disrupted β2M gene and/or a disrupted CD70 gene.

Any of the disrupted TRAC gene, the disrupted β2M gene, the disrupted CD70 gene, the disrupted Reg1 gene, and the disrupted TGFBRII gene can be genetically edited by a CRISPR/Cas-mediated gene editing system. For example, the disrupted TRAC gene may be genetically edited by a CRISPR/Cas-mediated gene editing system, which may comprise a gRNA comprising the nucleotide sequence of SEQ ID NO: 3. In another example, the disrupted β2M gene may be genetically edited by a CRISPR/Cas-mediated gene editing system, which may comprise a gRNA comprising the nucleotide sequence of SEQ ID NO: 7. In yet another example, the disrupted CD70 gene may be genetically edited by a CRISPR/Cas-mediated gene editing system, which may comprise a gRNA comprising the nucleotide sequence of SEQ ID NO: 11. In some examples, the disrupted Reg1 gene may be genetically edited by a CRISPR/Cas-mediated gene editing system, which may comprise a gRNA comprising the nucleotide sequence of SEQ ID NO: 337. In some examples, the disrupted TGFBRII gene may be genetically edited by a CRISPR/Cas-mediated gene editing system, which may comprise a gRNA comprising the nucleotide sequence of SEQ ID NO: 393.

In any of the genetically engineered T cells, the CAR may comprise an extracellular antigen binding domain specific to a tumor antigen, a co-stimulatory signaling domain of 4-1BB or CD28, and a cytoplasmic signaling domain of CD3ζ. In some embodiments, the population of genetically engineered T cells may comprise the disrupted TRAC gene and the disrupted β2M gene. In some instances, the genetically engineered T cells may further comprise the disrupted CD70 gene.

In some instances, the tumor antigen is B-cell maturation antigen (BCMA). In that case, the extracellular antigen binding domain is a single chain variable fragment (scFv) that binds BCMA. In some examples, the anti-BMCA scFv may comprise the amino acid sequence of SEQ ID NO: 277. In some specific examples, the anti-BCMA CAR may comprise the amino acid sequence of SEQ ID NO: 275, for example, comprising the amino acid sequence of SEQ ID NO: 274. In some instances, the anti-BCMA CAR may be encoded by the nucleotide sequence of SEQ ID NO: 273.

In other instances, the tumor antigen may be CD70. The extracellular antigen binding domain may be a single chain variable fragment (scFv) that binds CD70. In some instances, the anti-CD70 scFv may comprise the amino acid sequence of SEQ ID NO: 268 or SEQ ID NO: 270. In specific examples, the anti-CD70 CAR may comprise the amino acid sequence of SEQ ID NO:266, e.g., comprising the amino acid sequence of SEQ ID NO:265.

Any of the genetically engineered T cells disclosed herein may be derived from primary T cells of one or more human donors. Such genetically engineered T cells may show cytokine-dependent growth, and/or enhanced cytotoxicity and/or persistence as compared to non-engineered T cell counterparts.

In other aspects the present disclosure provides a method for preparing the population of genetically engineered T cells as disclosed herein. The method may comprise: (a) providing a plurality of cells, which are T cells or precursor cells thereof; (b) genetically editing a cbl-b gene of the T cells or the precursor cells thereof; and (c) producing the population of genetically engineered T cells having a disrupted cbl-b gene. In some embodiments, step (b) can be performed by delivering to the plurality of cells an RNA-guided nuclease and a gRNA targeting the cbl-b gene. Any of the gRNAs targeting the cbl-b gene ad disclosed herein can be used in the method for disrupting the cbl-b gene.

In any of the methods disclosed herein, the T cells of step (a) can be derived from primary T cells of one or more human donors. In some embodiments, the plurality of T cells in step (a) comprises one or more of the following genetic modifications: (i) engineered to express a chimeric antigen receptor (CAR); (ii) has a disrupted T cell receptor alpha chain constant region (TRAC) gene; (iii) has a disrupted β2M gene; and (iv) has a disrupted CD70 gene. In some embodiments, the plurality of T cells in step (a) may comprise one or more of the following genetic modifications: (v) has a disrupted Reg1 gene; and (vi) has a disrupted TGFBRII gene

In other embodiments, the method disclosed herein further comprises:
(i) delivering to the T cells a nucleic acid encoding a chimeric antigen receptor (CAR);
(ii) genetically editing a TRAC gene to disrupt its expression;
(iii) genetically editing a β2M gene to disrupt its expression;
(iv) genetically editing a CD70 gene to disrupt its expression; or
(v) a combination thereof.

In some instances, one or more of (ii)-(v) (e.g., all of (ii)-(vi)) are performed by one or more CRISPR/Cas-mediated gene editing systems comprising one or more RNA-guided nucleases and one or more gRNAs targeting the TRAC gene, the β2M gene, and/or the CD70 gene. In some examples, the gRNA targeting the TRAC gene comprises the nucleotide sequence of SEQ ID NO: 3. In some examples, the gRNA targeting the β2M gene comprises the nucleotide sequence of SEQ ID NO: 7. In some examples, the gRNA targeting the CD70 gene comprises the nucleotide sequence of SEQ ID NO: 11.

In some embodiments, the method disclosed herein may further comprise: (vi) genetically editing a Reg1 gene to disrupt its expression; (vii) genetically editing a TGFBRII gene to disrupt its expression; or (viii) a combination thereof. (vi) and/or (vii) may be performed by one or more CRISPR/Cas-mediated gene editing systems comprising one or more RNA-guided nucleases and one or more gRNAs targeting the Reg1 gene and/or the TGFBRII gene. For example, the gRNA targeting the Reg1 gene may comprise the nucleotide sequence of SEQ ID NO: 337. In another example, the gRNA targeting the TGFBRII gene may comprise the nucleotide sequence of SEQ ID NO: 393.

In some instances, the method may comprise delivering to the T cells or the precursor cells thereof one or more ribonucleoprotein particles (RNP), which comprises the RNA-guided nuclease, and one or more of the gRNAs. In some examples, the RNA-guided nuclease is a Cas9 nuclease. In a specific example, the Cas9 nuclease is a S. pyogenes Cas9 nuclease.

In some embodiments, the nucleic acid encoding the CAR can be in an AAV vector (e.g., an AAV6 vector). In some examples, the nucleic acid encoding the CAR may comprise a left homology arm and a right homology arm flanking the nucleotide sequence encoding the CAR. The left homology arm and the right homology arm are homologous to a genomic locus in the T cells, allowing for insertion of the nucleic acid into the genomic locus. In one specific example, the genomic locus is in the TRAC gene. In some examples, the method disclosed herein may comprise disrupting the TRAC gene by a CRISPR/Cas-mediated gene editing system comprising the gRNA that comprises the nucleotide sequence of SEQ ID NO: 3 and the nucleic acid encoding the CAR is inserted at the site targeted by the gRNA.

In some examples, the method comprises delivering to the T cells a nucleic acid encoding a CAR, which is specific to CD70, and genetically editing the CD70 gene to disrupt its expression.

Also provided herein is a population of genetically engineered T cells, which is prepared by any of the methods disclosed herein.

Further, the instant disclosure provides a method for eliminating undesired cells in a subject, the method comprising administering to a subject in need thereof genetically engineered T cells expressing a disrupted cbl-b gene and a chimeric antigen receptor targeting the undesired cells. Any of the populations of genetically engineered T cells disclosed here in can be used in the treatment method provided herein. In some embodiments, the undesired cells are cancer cells. In some embodiments, the subject is a human patient suffering from a cancer, for example, CD70+ cancer or a BCMA+ cancer. In some examples, the subject is a human patient suffering from a hematologic cancer. In other examples, the subject is a human patient suffering from a solid tumor.

Also provided herein are any of the genetically engineered T cells disclosed herein or a pharmaceutical composition comprising such for use in treating cancer, as well as uses of the genetically engineered T cells for manufacturing a medicament for the intended therapeutic purposes.

In addition, the present disclosure provides a guide RNA (gRNA) targeting a cbl-b gene, comprising a nucleotide sequence specific to a fragment in exon 2, exon 7, exon 9, exon 11, or exon 12 of the cbl-b gene. In some examples, the gRNA comprises a nucleotide sequence specific to exon 2 of the cbl-b gene. In some examples, the gRNA comprises a nucleotide sequence specific to exon 7 of the cbl-b gene. In some examples, the gRNA comprises a nucleotide sequence specific to exon 9 of the cbl-b gene.

In some embodiments, the gRNA targeting the cbl-b gene may comprise a spacer having the nucleotide sequence of SEQ ID NOs: SEQ ID NOs: 25, 29, 33, 37, 41, 45, 49, 53, 57, 61, 65, 69, 73, 77, or 81. In some examples, the gRNA targeting the cbl-b gene comprises a spacer of SEQ ID NO:41. In some examples, the gRNA targeting the cbl-b gene comprises a spacer of SEQ ID NO: 33. In some examples, the gRNA targeting the cbl-b gene comprises a spacer of SEQ ID NO:49. In some examples, the gRNA targeting the cbl-b gene comprises a spacer of SEQ ID NO:65. In some examples, the gRNA targeting the cbl-b gene comprises a spacer of SEQ ID NO:69.

In some examples, the gRNA targeting the cbl-b gene further comprises a scaffold sequence. Alternatively, or in addition, the gRNA comprises one or more modified nucleotides. For example, the gRNA may comprise one or more 2′-O-methyl phosphorothioate residues at the 5′ and/or 3′ terminus of the gRNA. Exemplary gRNAs targeting the cbl-b gene ma comprise the nucleotide sequence of SEQ ID NO: SEQ ID NOs: 24, 28, 32, 36, 40, 44, 48, 52, 56, 60, 64, 68, 72, 76, or 80. In one example, the gRNA is CBLB-T3. In another example, the gRNA is CBLB-6. In yet other examples, the gRNA is CBLB-8. Alternatively, the gRNA is CBLB-12. In yet another example, the gRNA is CBLB-13.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1B include diagrams showing screening of gRNAs targeting the cbl-b gene. FIG. 1A: CBLB protein levels in cells treated by three gRNAs as indicated. FIG. 1B: CBLB protein levels in cells treated by various gRNAs as indicated. Effective protein knock-down was observed in cells treated by certain gRNAs (see, e.g., those indicated by the arrows).

FIGS. 2A-2B include diagrams shows cytotoxicity effects of TRAC-/β2M-/CD70-/Cblb-/anti-CD70 CAR+ cells. FIG. 2A: cytotoxicity of the TRAC-/β2M-/CD70- anti-CD70 CAR+ cells and TRAC-/β2M-/CD70-/CBLB- anti-CD70 CAR+ (anti-CD70 CAR+CBLB KO) on A498 cells. FIG. 2B: cytotoxicity of anti-CD70 CAR+ T cells cells and Anti-CD70+CBLB KO cells on Caki-1 cells.

FIGS. 3A-3B include diagrams showing cytokine secretion in TRAC-/β2M-/CD70-/CBLB- anti-CD70 CAR+ cells (Anti-CD70+CBLB) and TRAC-/β2M-/CD70-anti-CD70 CAR+ cells (Anti-CD70) after co-culture with Caki-1 cells. FIG. 3A: IFN-γ secretion FIG. 3B: IL-2 secretion.

FIG. 4 is a graph showing the number of anti-CD70 CAR+ cells, with or without cbl-b gene disruption, in the blood of mice transplanted with Caki-1 RCC tumors as determined by flow cytometric analysis. Disruption of the cbl-b gene significantly increases CAR-T cells persistence in vivo as compared to the wild-type cbl-b counterpart cells.

FIGS. 5A-5B include diagrams showing consistent CRISPR/Cas editing in anti-BCMA CAR-T as determined by FACS. FIG. 5A: TRAC-/β2M- anti-BCMA CAR+ T-cells, TRAC-/β2M-/CBLB- anti-BCMA CAR+ T-cells, and TRAC-/β2M- T-cells with no CAR have consistent rates of β2m and TRAC KO, at >60% and >90%, respectively FIG. 5B: TRAC-/β2M- anti-BCMA CAR+ T-cells and TRAC-/β2M-/CBLB- anti-BCMA CAR+ T-cells have consistent rates of CAR insertion (>70%), while the TRAC-/β2M- T-cells with no CAR have no detectable staining. Anti-BCMA CAR expression was measured by determining the percent of cells that bound biotinylated BCMA subsequently detected by FACS using streptavidin-APC.

FIG. 6 is photo showing that anti-BCMA CAR-T cells edited to disrupt the CBLB locus have severely reduced CBLB expression as determined by a Western blot analysis. The Westernblot analysis was performed using protein lysates from TRAC-/β2M-/CBLB- anti-BCMA CAR+ T-cells and unedited T-cells from the same donor, showing that unedited T-cells show strong CBLB expression as a thick band at ~ 109kDa, whereas the corresponding band for the TRAC-/β2M-/CBLB- anti-BCMA CAR+ T-cells is extremely faint, indicating highly reduced expression.

FIGS. 7A-7B include chartsshows enhanced cytotoxicity of the engineered TRAC-/β2M-/CBLB- anti-BCMA CAR+ T-cells as relative to the CBLB wild-type counterpart cells. FIG. 7A: againstMM.1S cells FIG. 7B: against JeKo-1 cells. TRAC-/β2M- T-cells with no CAR show minimal cytotoxicity to either cell line.

FIGS. 8A-8F include charts showing cytokine secretion in the engineered T-cells as indicated. FIG. 8A: IFN-γ secretion when co-cultured with MM.1S. FIG. 8B: IFN-γ secretion co-cultured with JeKo-1. FIG. 8C: Granzyme A secretion when co-cultured with MM.1S. FIG. 8D: Granzyme A secretion when co-cultured with JeKo-1. FIG. 8E: TNF-α secretion when co-cultured with MM.1S. FIG. 8F: TNF-α secretion when co-cultured with JeKo-1.

FIGS. 9A-9C include graphs showing superior efficacy of TRAC-/β2M-/CBLB- anti-BCMA CAR+ T-cells relative to TRAC-/β2M- anti-BCMA CAR+ T-cells in a multiple myeloma murine model. NOG mice were implanted with 10e6 RPMI-8226 cells and injected with either 0.35e6 or 1e6 CAR-T cells ten days later. FIG. 9A: survival curve. FIG. 9B: tumor volumes in the animals injected with 1e6 CAR+ T-cells. FIG. 9C: tumor volumes in the animals injected with 0.35e6 CAR+ T-cells. Both doses TRAC-/β2M-/CBLB- anti-BCMA CAR+ T-cells regressed tumor size for the majority of the study, unlike either dose of TRAC-/β2M- anti-BCMA CAR+ T-cells.

### DETAILED DESCRIPTION OF THE INVENTION

The present disclosure aims at establishing genetically engineered T cells having enhanced cytotoxicity, persistence, improved growth activity, reduced T cell exhaustion, and enhanced potency, a long-felt need in CAR-T therapy. Such a T cell may use bona fide T cells as the starting material, for example, non-transformed T cells, terminally differentiated T cells, T cells having stable genome, and/or T cells that depend on cytokines and growth factors for proliferation and expansion. Alternatively, such a T cell may use T cells generated from precursor cells such as hematopoietic stem cells (e.g., iPSCs), e.g., in vitro culture. The T cells disclosed herein may confer one or more benefits in both CAR-T cell manufacturing and clinical applications.

Conventional allogenic CAR T cells are produced wherein a single donor leukopak is edited in most cases so that the cells can avoid components of the patient immune system and thus do not cause GvHD. The process of expanding these CAR T cells can yield 10s to 100s of vialed drug product. Patients may receive a single dose or multiple doses. During the manufacturing process, these CAR T cells lose potential due to various mechanisms, for example, apoptosis, exhaustion, replicative senescence, and other processes where the cells become less fit.

The genetically engineered T cells having a disrupted cbl-b gene and optionally one or more additional genetic edits, for example, a disrupted TRAC gene, a disrupted β2M gene, a disrupted CD70 gene, a disrupted Reg1 gene, a disrupted TGFBRII gene, and/or an inserted nucleic acid coding for a chimeric antigen receptor (CAR), or a combination thereof.

Unexpectedly, the present disclosure reports that disrupting cbl-b in T cells, either taken alone or in combination of disruptions of Reg1 and TGFBRII genes, led to various advantageous features in T cell-mediated cell therapy such as CAR-T therapy. Examples include but are not limited to improved cytotoxicity, higher viability, and persistence in vivo. These features are beneficial for manufacturing and production of therapeutic T-cell based products such as CAR-T cells; T cell potency advantages related to maintaining therapeutic T cells (e.g., CAR-T cells); in vitro and in vivo potency and activity (target cell killing) for a more effective and persistent T-cell based therapeutic products.

Moreover, CAR-T cells having a disrupted cbl-b gene showed much higher anti-tumor activities in animal models as relative to CAR-T cells. This offers unlimited advantageous features of the engineered T cells provided herein including:
(a) Enhanced cytotoxicity.
(b) Increased persistence in vivo.
(c) Enhanced anti-tumor activity, e.g., reduction of tumor size and/or elongated survival rates.

Accordingly, provided herein are T cells having improved persistence in culture, methods of producing such T cells, and methods of using such T cells for producing therapeutic T cells such as CAR-T cells. Components and processes (e.g., the CRISPR approach for gene editing and components used therein) for making the T cells disclosed herein are also within the scope of the present disclosure.

### I. Genetically Engineered T Cells Having Enhanced Features

The T cells disclosed herein comprises genetically engineered T cells having enhanced persistence in culture. Such genetically engineered T cells have genetic editing of the cbl-b gene. In some instances, such genetically engineered T cells may have additional gene edits, for example, a disrupted CD70 gene, a disrupted TRAC gene, a disrupted β2M gene, a disrupted Reg1 gene, a disrupted TGFBRII gene, or a combination thereof. The genetically engineered T cells disclosed herein may further be engineered to express a chimeric antigen receptor (CAR) as disclosed herein.

The genetically engineered T cells disclosed herein, having a disrupted cbl-b gene, either alone or in combination with the additional gene edits also disclosed herein, show one or more of the following superior features as relative to the T cell counterparts having a wild-type cbl-b gene: enhanced cytotoxicity, enhanced longevity, and enhanced potency.

The genetically engineered T cells may be derived from parent T cells (e.g., non-edited wild-type T cells) obtained from a suitable source, for example, one or more mammal donors. In some examples, the parent T cells are primary T cells (e.g., non-transformed and terminally differentiated T cells) obtained from one or more human donors. Alternatively, the parent T cells may be differentiated from precursor T cells obtained from one or more suitable donor or stem cells such as hematopoietic stem cells or inducible pluripotent stem cells (iPSC), which may be cultured in vitro.

Also within the scope of the present disclosure are other types of genetically engineerd immune cells (e.g., natural killer cells or NK cells) having a disrupted cbl-b gene, and optionally additional gene edits, for example, one or more disrupted endogenous genes, one or more knock-in transgenes (e.g., a CAR-encoding transgene), or a combination thereof.

In some embodiments, the genetically engineered T cells carry a disrupted cbl-b gene, and optionally, one or more disrupted genes involved in cell exhaustion (e.g., CD70) or other cellular pathways to improve CAR-T cell functionality (e.g., Reg1 and/or TGFBRII). Such genetically engineered T cells may further comprise one or more disrupted genes, for example, TRAC or β2M. Such genetically engineered T cells may further express a chimeric antigen receptor (CAR), which may be capable of binding to an antigen of interest, for example, a tumor associated antigen (e.g., BCMA or CD70).

In some embodiments, the genetically engineered T cells carry a disrupted cbl-b gene and a disrupted CD70 gene. Such genetically engineered T cells may further comprise one or more disrupted genes, for example, TRAC and/or β2M. In some instances, the genetically engineered T cells may further comprise one or more disrupted genes, for example, Reg1 and/or TGFBRII. The genetically engineered T cells disclosed herein may further express a chimeric antigen receptor (CAR), which may be capable of binding to an antigen of interest, for example, a tumor associated antigen (e.g., BCMA or CD70). In some instances, the genetically T cells may express a CAR that does not bind CD70.

Any of the genetically engineered T cells may be generated via gene editing (including genomic editing), a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

### (A) Genetically Edited Genes

In some aspects, the present disclosure provides genetically engineered T cells (e.g., CAR-T cells) that may comprise a disrupted cbl-b gene, and optionally one or more of the additional gene edits as also disclosed herein, e.g., a disrupted CD70 gene, a disrupted β2M gene, a disrupted TRAC gene, a disrupted Reg1 gene, a disrupted TGFBRII gene, or a combination thereof.

As used herein, a “disrupted gene” refers to a gene comprising an insertion, deletion, or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell.

### Cbl-b Gene Editing

In some embodiments, the genetically engineered T cells may comprise a disrupted Cbl proto-oncogene B (cbl-b) gene. The CBLB protein contains a zinc finger motif, binds RNA and exhibits ribonuclease activity. CBLB plays roles in both immune and non-immune cells and its expression can be rapidly induced under diverse conditions including microbial infections, treatment with inflammatory cytokines and chemical or mechanical stimulation. Human cbl-b gene is located on chromosome GRCh38.p13. Additional information can be found in GenBank under Gene ID: 868.

In some examples, the genetically engineered T cells may comprise a disrupted cbl-b gene such that the expression of cbl-b in the T cells is substantially reduced or eliminated completely. The disrupted cbl-b gene may comprise one or more genetic edits at one or more suitable target sites (e.g., in coding regions or in non-coding regulatory regions such as promoter regions) that disrupt expression of the cbl-b gene. Such target sites may be identified based on the gene editing approach for use in making the genetically engineered T cells. Exemplary target sites for the genetic edits may include exon 2, exon 7, exon 9, exon 11, exon 12, or a combination thereof. In some examples, one or more genetic editing may occur in exon 2. In other examples, one or more genetic editing may occur in exon 7. In yet other examples, one or more genetic editing may occur in exon 9. Such genetic editing may be induced by the CRISPR/Cas technology using a suitable guide RNA, for example, those listed in Table 2. The resultant edited cbl-b gene using a gRNA listed in Table 2 may comprise one or more edited sequences provided in Tables 3-11 below. The genetically engineered T cells disclosed herein may comprise a disrupted cbl-b gene having one or more of the edited sequences provided in Tables 3-11.

### CD70 Gene Editing

CD70 is a gene involved in T cell exhaustion, which is a process of stepwise and progressive loss of T cell functions. T cell exhansuion may be induced by prolonged antigen stimulation or other factors. Genes involved in T cell exhaustion refer to those that either positively regulate or negatively regulate this biological process. The genetically engineered T cells disclosed herein may comprise genetic editing of a gene that positively regulates T cell exhaustion to disrupt its expression. Alternatively, or in addition, the genetically engineered T cells may comprise genetic editing of a gene that negatively regulates T cell exhaustion to enhance its expression and/or biologic activity of the gene product.

In some embodiments, the genetically engineered T cells may comprise an edited gene involved in T cell exhaustion, e.g., disruption of a gene that positively regulates T cell exhaustion. Such a gene may be a Cluster of Differentiation 70 (CD70) gene. CD70 is a member of the tumor necrosis factor superfamily and its expression is restricted to activated T and B lymphocytes and mature dendritic cells. CD70 is implicated in tumor cell and regulatory T cell survival through interaction with its ligand, CD27. CD70 and its receptor CD27 have multiple roles in immune function in multiple cell types including T cells (activated and T_{reg} cells), and B cells.

It was also found that disrupting the CD70 gene in immune cells engineered to express an antigen targeting moiety enhanced anti-tumor efficacy against large tumors and induced a durable anti-cancer memory response. Specifically, the anti-cancer memory response prevented tumor growth upon re-challenge. Further, it has been demonstrated disrupting the CD70 gene results in enhanced cytotoxicity of immune cells engineered to express an antigen targeting moiety at lower ratios of engineered immune cells to target cells, indicating the potential efficacy of low doses of engineered immune cells. See, e.g., WO2019/215500, the relevant disclosures of which are incorporated by reference for the purpose and subject matter referenced herein.

Structures of CD70 genes are known in the art. For example, human CD70 gene is located on chromosome 19p13.3. The gene contains four protein encoding exons. Additional information can be found in GenBank under Gene ID: 970.

In some examples, the genetically engineered T cells may comprise a disrupted CD70 gene such that the expression of CD70 in the T cells is substantially reduced or eliminated completely. The disrupted CD70 gene may comprise one or more genetic edits at one or more suitable target sites (e.g., in coding regions or in non-coding regulatory regions such as promoter regions) that disrupt expression of the CD70 gene. Such target sites may be identified based on the gene editing approach for use in making the genetically engineered T cells. Exemplary target sites for the genetic edits may include exon 1, exon 2, exon 3, exon 4, or a combination thereof. See also WO2019/215500, the relevant disclosures of which are incorporated by reference for the purpose and subject matter referenced herein.

In some embodiments, the gRNA targeting CD70 listed in Table 1 may be used for disrupting the CD70 gene via CRISPR/Cas9 gene editing.

### β2M Gene Edit

In some embodiments, the genetically engineered T cells disclosed herein may further comprise a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response. It is known to those skilled in the art that different nucleotide sequences in an edited gene such as an edited β2M gene may be generated by a single gRNA such as the one listed in Table 1 (e.g., SEQ ID NO: 5 or 6). See also WO2019097305, the relevant disclosures of which are incorporated by reference for the subject matter and purpose referenced herein.

The genetically engineered T cells disclosed herein may further comprise one or more additional gene edits (e.g., gene knock-in or knock-out) to improve T cell function. Examples include knock-in or knock-out genes to improve target cell lysis, knock-in or knock-out genes to enhance performance of therapeutic T cells such as CAR-T cells prepared from the genetically engineered T cells.

### TRAC Gene Edit

In some embodiments, the genetically engineered T cells as disclosed herein may further comprise a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. See also WO2019097305, the relevant disclosures of which are incorporated by reference herein for the purpose and subject matter referenced herein. It is known to those skilled in the art that different nucleotide sequences in an edited gene such as an edited TRAC gene may be generated by a single gRNA such as the one listed in Table 1 (e.g., SEQ ID NO: 1 or 2).

It should be understood that more than one suitable target site/gRNA can be used for each target gene disclosed herein, for example, those known in the art or disclosed herein. Additional examples can be found in, e.g., WO2019097305, the relevant disclosures of which are incorporated by reference herein for the purpose and subject matter referenced herein.

The genetically engineered CAR-T cells having a disrupted cbl-b gene disclosed herein have advantageous features relative to CAR-T cells having a wild-type cbl-b (counterpart T cells). For example, disruption of the cbl-b gene led to enhanced target cell killing and enhanced anti-tumor effects, for example, a reduction of tumor size and/or extended survival times as observed in xenograft mouse models. Further, CAR-T cells having a disrupted cbl-b gene show enhanced persistence as relative to the cbl-b wild-type counterpart. “T cell persistence” as used herein refers to the tendency of T cells to continue to grow, proliferate, self-renew, expand, and maintain healthy activity. In some instances, T cell persistence can be represented by the longevity of the T cells, which can be measured by conventional methods and/or assays described herein.

### Reg1 Gene Editing

In some embodiments, the genetically engineered T cells may comprise a disrupted gene involved in mRNA decay. Such a gene may be Reg1. Reg1 contains a zinc finger motif, binds RNA and exhibits ribonuclease activity. Reg1 plays roles in both immune and non-immune cells and its expression can be rapidly induced under diverse conditions including microbial infections, treatment with inflammatory cytokines and chemical or mechanical stimulation. Human Reg1 gene is located on chromosome 1p34.3. Additional information can be found in GenBank under Gene ID: 80149.

In some examples, the genetically engineered T cells may comprise a disrupted Reg1 gene such that the expression of Reg1 in the T cells is substantially reduced or eliminated completely. The disrupted Reg1 gene may comprise one or more genetic edits at one or more suitable target sites (e.g., in coding regions or in non-coding regulatory regions such as promoter regions) that disrupt expression of the Reg1 gene. Such target sites may be identified based on the gene editing approach for use in making the genetically engineered T cells. Exemplary target sites for the genetic edits may include exon 1, exon 2, exon 3, exon 4, exon 5, exon 6, or a combination thereof. In some examples, one or more genetic editing may occur in exon 2 or exon 4. Such genetic editing may be induced by the CRISPR/Cas technology using a suitable guide RNA, for example, those listed in Table 20. See also WO2022/064428, the relevant disclosures of which are incorporated by reference for the subject matter and purpose referenced herein.

### TGFBRII Gene Editing

In some embodiments, the genetically engineered T cells may comprise a disrupted TGFBRII gene, which encodes Transforming Growth Factor Receptor Type II (TGFBRII). TGFBRII receptors are a family of serine/threonine kinase receptors involved in the TGFβ signaling pathway. These receptors bind growth factor and cytokine signaling proteins in the TGFβ family, for example, TGFβs (TGFβ1, TGFβ2, and TGFβ3), bone morphogenetic proteins (BMPs), growth differentiation factors (GDFs), activin and inhibin, myostatin, anti-Müllerian hormone (AMH), and NODAL.

In some examples, the genetically engineered T cells may comprise a disrupted TGFBRII gene such that the expression of TGFBRII in the T cells is substantially reduced or eliminated completely. The disrupted TGFBRII gene may comprise one or more genetic edits at one or more suitable target sites (e.g., in coding regions or in non-coding regulatory regions such as promoter regions) that disrupt expression of the TGFBRII gene. Such target sites may be identified based on the gene editing approach for use in making the genetically engineered T cells. Exemplary target sites for the genetic edits may include exon 1, exon 2, exon 3, exon 4, exon 5, or a combination thereof. In some examples, one or more genetic editing may occur in exon 4 and/or exon 5. Such genetic editing may be induced by a gene editing technology, (e.g., the CRISPR/Cas technology) using a suitable guide RNA, for example, those listed in Table 21. See also WO2022/064428, the relevant disclosures of which are incorporated by reference for the subject matter and purpose referenced herein.

### (C) Methods of Making Genetically Engineered T Cells

The genetically engineered T cells disclosed herein can be prepared by genetic editing of parent T cells or precursor cells thereof via a conventional gene editing method or those described herein.

### (I) T Cells

In some embodiments, T cells can be derived from one or more suitable mammals, for example, one or more human donors. T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some examples, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRab, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, subpopulations of T cells may be isolated by positive or negative selection prior to genetic engineering and/or post genetic engineering.

An isolated population of T cells may express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In some instances, the T cell population comprises primary T cells isolated from one or more human donors. Such T cells are terminally differentiated, not transformed, depend on cytokines and/or growth factors for growth, and/or have stable genomes.

Alternatively, the T cells may be derived from stem cells (e.g., HSCs or iPSCs) via in vitro differentiation.

T cells from a suitable source can be subjected to one or more rounds of stimulation, activation and/or expansion. T cells can be activated and expanded generally using methods as described, for example, in U.S. Patents 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some instances, the T cell population can be expanded and/or activated after the genetic editing as disclosed herein. T cell populations or isolated T cells generated by any of the gene editing methods described herein are also within the scope of the present disclosure.

### (Ii) Gene Editing Methods

Any of the genetically engineered T cells can be prepared using conventional gene editing methods or those described herein to edit one or more of the target genes disclosed herein (targeted editing). Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions, or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer DE et al. Vis. Exp. 2015; 95:e52118).

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration. Some exemplary approaches are disclosed in detail below.

### CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and transactivating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically < 20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

### Endonuclease for Use in CRISPR

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). One example is provided in Table 1 below.

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

### Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20-nucleotide sequence) that defines the target sequence (e.g., a DNA target sequences, such as a genomic target sequence) of a target gene of interest. In some embodiments, the spacer sequence ranges from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence“ is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. For example, if the target sequence is 5′-AGAGCAACAGTGCTGTGGCC**-3′ (SEQ ID NO: 14), then the gRNA spacer sequence is 5′-AGAGCAACAGUGCUGUGGCC**-3′ (SEQ ID NO: 3). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be an sgRNA, which may comprise a 20-nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence. Examples are provided in Table 1 below. In these exemplary sequences, the fragment of “n” refers to the spacer sequence at the 5′ end.

In some embodiments, the sgRNA comprises comprise no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a cbl-b gene, for example, target a site within exon 2, exon 7, exon 9, exon 11, or exon 12 of the cbl-b gene. Such a gRNA may comprise a spacer sequence complementary (complete or partially) to the target sequences in exon 2 of a cbl-b gene, or a fragment thereof. In other examples, a gRNA may comprise a spacer sequence complementary (complete or partially) to the target sequences in exon 7 of a cbl-b gene, or a fragment thereof. Alternatively, a gRNA may comprise a spacer sequence complementary (complete or partially) to the target sequences in exon 9 of a cbl-b gene, or a fragment thereof. Exemplary target sequences in a cbl-b gene and exemplary gRNA sequences are provided in Table 2 below.

In some embodiments, the gRNAs disclosed herein target a CD70 gene, for example, target a site within exon 1 or exon 3 of a CD70 gene. Such a gRNA may comprise a spacer sequence complementary (complete or partially) to the target sequences in exon 1 or exon 3 of a CD70 gene, or a fragment thereof. Exemplary target sequences in a CD70 gene and exemplary gRNAs specific to the CD70 gene are provided in Table 1 below.

In some embodiments, the gRNAs disclosed herein target a β2M gene, for example, target a suitable site within a β2M gene. See also WO2019097305, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein. Exemplary spacer sequences and gRNAs targeting a β2M gene are provided in Table 1 below.

In some embodiments, the gRNAs disclosed herein target a TRAC gene. See also WO2019097305, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154. Ensembl; ENSG00000277734). In some embodiments, gRNAs targeting the TRAC genomic region and RNA-guided nuclease create breaks in the TRAC genomic region resulting Indels in the TRAC gene disrupting expression of the mRNA or protein.

Exemplary spacer sequences and gRNAs targeting a TRAC gene are provided in Table 1 below.

In some embodiments, the gRNAs disclosed herein target a Reg1 gene, for example, target a site within exon 1, exon 2, exon 3, exon 4, exon 5, or exon 6 of the Reg1 gene. Such a gRNA may comprise a spacer sequence complementary (complete or partially) to the target sequences in exon 2 or exon 4 of a Reg1 gene, or a fragment thereof. Exemplary target sequences of Reg1 and exemplary gRNA sequences are provided in Table 20 below. In one example, the gRNA targeting a Reg1 gene is specific to a target sequence of SEQ ID NO: 358. Such a gRNA may comprise a spacer sequence of SEQ ID NO: 337. In specific examples, the gRNA targeting a Reg1 gene may comprise the nucleotide sequence of SEQ ID NO: 335, either unmodified or modified such as described in SEQ ID NO: 336.

In some embodiments, the gRNAs disclosed herein target a TGFBRII gene, for example, target a site within exon 1, exon 2, exon 3, exon 4, exon 5, or exon 6 of the TGFBRII gene. Such a gRNA may comprise a spacer sequence complementary (complete or partially) to the target sequences in exon 4 or exon 5 of a TGFBRII gene, or a fragment thereof. Exemplary target sequences of TGFBRII and exemplary gRNA sequences are provided in Table 21 below. In one example, the gRNA targeting a TGFBRII gene is specific to a target sequence of SEQ ID NO: 412. Such a gRNA may comprise a spacer sequence of SEQ ID NO: 393. In specific examples, the gRNA targeting a TGFBRII gene may comprise the nucleotide sequence of SEQ ID NO: 391, either unmodified or modified such as described in SEQ ID NO: 392.

By way of illustration, guide RNAs used in the CRISPR/Cas/Cpf1 system, or other smaller RNAs can be readily synthesized by chemical means, as illustrated below, and described in the art. While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some examples, the gRNAs of the present disclosure can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In certain embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments of the present disclosure, a CRISPR/Cas nuclease system for use in genetically editing any of the target genes disclosed here may include at least one guide RNA. In some examples, the CRISPR/Cas nuclease system may contain multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

### Delivery of Guide RNAs and Nucleases to T Cells

The CRISPR/Cas nuclease system disclosed herein, comprising one or more gRNAs and at least one RNA-guided nuclease, optionally a donor template as disclosed below, can be delivered to a target cell (e.g., a T cell) for genetic editing of a target gene, via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, gRNA and a RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and one or more gRNAs targeting one or more genes of interest can be delivered a cell (e.g., a T cell). In some embodiments, an RNP can be delivered to a T cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively, or in addition, a gRNA targeting a gene can be delivered to a cell as a RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used.

### Other Gene Editing Methods

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. No. 7,888,121 and U.S. Pat. No. 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

### II. Genetically Engineered T Cells Expression a Chimeric Antigen Receptor (CAR)

The genetically engineered T cells having a disrupted cbl-b gene, or a combination of disrupted cbl-b gene and disrupted CD70 gene. Optionally, such genetically engineered T cells may comprise one or more of additional disrupted genes, e.g., β2M, TRAC, Reg1, TGFBRII, or a combination thereof as disclosed herein, may further express a chimeric antigen receptor (CAR) targeting an antigen of interest or cells expressing such an antigen.

### (A) Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include SEQ ID NO: 241 and SEQ ID NO: 242 as provided in Table 13 below. Other signal peptides may be used.

### (I) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen such as a tumor antigen. In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens, or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

In some embodiments, the antigen-binding extracellular domain can be a single-chain variable fragment (scFv) that binds a tumor antigen as disclosed herein. The scFv may comprise an antibody heavy chain variable region (V_{H}) and an antibody light chain variable region (V_{L}), which optionally may be connected via a flexible peptide linker. In some instances, the scFv may have the V_{H} to V_{L} orientation (from N-terminus to C-terminus). Alternatively, the scFv may have the V_{L} to V_{H} orientation (from N-terminus to C-terminus).

Exemplary tumor antigens include, but are not limited to, CD19, BCMA, CD70, CD33, and PTK7. Any known antibodies specific to such tumor antigens, for example, those approved for marketing and those in clinical trials, can be used for making the CAR constructs disclosed herein. Non-limiting examples of CAR constructs are provided in WO2019097305 and WO2019215500, WO2020/095107, and International Patent Application No. PCT/IB2021/053849, the relevant disclosures of which are herein incorporated by reference for the purposes and subject matter referenced herein.

In some examples, the antigen-binding extracellular domain can be a single-chain variable fragment (scFv) that binds human CD70. In some instances, the anti-CD70 scFv may comprises (i) a heavy chain variable region (V_{H}) that comprises the same heavy chain complementary determining regions (CDRs) as those in SEQ ID NO: 271; and (ii) a light chain variable region (V_{L}) that comprises the same light chain CDRs as those in SEQ ID NO: 272. In some specific examples, the anti-CD70 antibody discloses herein may comprise the heavy chain CDR1, heavy chain CDR2, and heavy chain CDR3 set forth as SEQ ID NOs: 259, 261, and 263, respectively as determined by the Kabat method. Alternatively, or in addition, the anti-CD70 antibody discloses herein may comprise the light chain CDR1, light chain CDR2, and light chain CDR3 set forth as SEQ ID NOs: 253, 255, and 257, respectively as determined by the Kabat method. Alternatively, the anti-CD70 antibody discloses herein may comprise the heavy chain CDR1, heavy chain CDR2, and heavy chain CDR3 set forth as SEQ ID NOs: 260, 262, and 264, respectively as determined by the Chothia method. Alternatively, or in addition, the anti-CD70 antibody discloses herein may comprise the light chain CDR1, light chain CDR2, and light chain CDR3 set forth as SEQ ID NO:254, LAS, and SEQ ID NO: 258, respectively as determined by the Chothia method. In one specific example, the anti-CD70 scFv may comprise a V_{H} comprising the amino acid sequence of SEQ ID NO: 271 and a V_{L} comprises the amino acid sequence of SEQ ID NO: 272. See Sequence Table 13 below.

In some examples, the antigen-binding extracellular domain can be a single-chain variable fragment (scFv) that binds human BCMA. In some instances, the anti-BCMA scFv may comprises (i) a heavy chain variable region (V_{H}) that comprises the same heavy chain complementary determining regions (CDRs) as those in SEQ ID NO: 278; and (ii) a light chain variable region (V_{L}) that comprises the same light chain CDRs as those in SEQ ID NO: 279. In some specific examples, the anti-BCMA antibody discloses herein may comprise the heavy chain CDR1, heavy chain CDR2, and heavy chain CDR3 set forth as SEQ ID NOs: 284, 286, and 288, respectively as determined by the Kabat method. Alternatively, or in addition, the anti-BCMA antibody discloses herein may comprise the light chain CDR1, light chain CDR2, and light chain CDR3 set forth as SEQ ID NOs: 280, 281, and 282, respectively as determined by the Kabat method. Alternatively, the anti-BCMA antibody discloses herein may comprise the heavy chain CDR1, heavy chain CDR2, and heavy chain CDR3 set forth as SEQ ID NOs: 285, 287, and 289, respectively as determined by the Chothia method. Alternatively, or in addition, the anti-BCMA antibody discloses herein may comprise the light chain CDR1, light chain CDR2, and light chain CDR3 set forth as SEQ ID NOs:280, 281, and 283, respectively as determined by the Chothia method. In one specific example, the anti-BCMA scFv may comprise a V_{H} comprising the amino acid sequence of SEQ ID NO: 278 and a V_{L} comprises the amino acid sequence of SEQ ID NO: 279. See Sequence Table 13 below.

In some examples, the antigen-binding extracellular domain can be a single-chain variable fragment (scFv) that binds human CD33. Exemplary anti-CD33 scFv and anti-CD33 CAR constructs can be found, for example, in Sequence Table 13 below and in WO2020/095107, the relevant disclosures of which are incorporated by reference for the subject matter and purpose noted herein.

In some examples, the anti-BCMA scFv may comprise a V_{H} comprising the amino acid sequence of SEQ ID NO: 278 and a V_{L} comprises the amino acid sequence of SEQ ID NO: 279. See Sequence Table 13 below.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/ or abysis.org/abysis/sequence_input).

### (II) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of SEQ ID NO: 243 as provided below in Table 13. Other transmembrane domains may be used.

### (III) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

### (IV) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

Table 13 provides examples of signaling domains derived from 4-1BB, CD28 and CD3-zeta that may be used herein.

In other examples, the anti-BCMA CAR disclosed herein may comprise the amino acid sequence of SEQ ID NO: 274, which may be encoded by the nucleotide sequence of SEQ ID NO: 273. Alternatively, the anti-BCMA CAR may be a mature form without the N-terminal signal peptide, e.g., comprising the amino acid sequence of SEQ ID NO:275.

In other examples, the anti-CD70 CAR disclosed herein may comprise the amino acid sequence of SEQ ID NO: 265, which may be encoded by the nucleotide sequence of SEQ ID NO: 267. Alternatively, the anti-CD70 CAR may be a mature form without the N-terminal signal peptide, e.g., comprising the amino acid sequence of SEQ ID NO: 266. See sequence Table 13 provided below.

### (B) Delivery of CAR Construct to T Cells

In some embodiments, a nucleic acid encoding a CAR can be introduced into any of the genetically engineered T cells disclosed herein by methods known to those of skill in the art. For example, a coding sequence of the CAR may be cloned into a vector, which may be introduced into the genetically engineered T cells for expression of the CAR. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

In specific examples, a nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions disclosed herein can be used for this purpose.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting a CAR coding segment into the TRAC gene.

In some embodiments, a nucleic acid encoding a CAR (e.g., via a donor template, which can be carried by a viral vector such as an adeno-associated viral (AAV) vector) can be designed such that it can insert into a location within a β2M gene to disrupt the β2M gene in the genetically engineered T cells and express the CAR polypeptide. Disruption of β2M leads to loss of function of the endogenous MHC Class I complexes. For example, a disruption in the β2M gene can be created with an endonuclease such as those described herein and one or more gRNAs targeting one or more β2M genomic regions. Any of the gRNAs specific to a β2M gene and the target regions disclosed herein can be used for this purpose.

In some examples, a genomic deletion in the β2M gene and replacement by a CAR coding segment can be created by homology directed repair or HDR (e.g., using a donor template, which may be part of a viral vector such as an adeno-associated viral (AAV) vector). In some embodiments, a disruption in the β2M gene can be created with an endonuclease as those disclosed herein and one or more gRNAs targeting one or more β2M genomic regions and inserting a CAR coding segment into the β2M gene.

In some embodiments, a nucleic acid encoding a CAR (e.g., via a donor template, which can be carried by a viral vector such as an adeno-associated viral (AAV) vector) can be designed such that it can insert into a location within a CD70 gene to disrupt the CD70 gene in the genetically engineered T cells and express the CAR polypeptide. Disruption of CD70 leads to loss of function of the endogenous CD70 protein. For example, a disruption in the CD70 gene can be created with an endonuclease such as those described herein and one or more gRNAs targeting one or more CD70 genomic regions. Any of the gRNAs specific to a CD70 gene and the target regions disclosed herein can be used for this purpose.

In some examples, a genomic deletion in the CD70 gene and replacement by a CAR coding segment can be created by homology directed repair or HDR (e.g., using a donor template, which may be part of a viral vector such as an adeno-associated viral (AAV) vector). In some embodiments, a disruption in the CD70 gene can be created with an endonuclease as those disclosed herein and one or more gRNAs targeting one or more CD70 genomic regions and inserting a CAR coding segment into the CD70 gene.

In some embodiments, a nucleic acid encoding a CAR (e.g., via a donor template, which can be carried by a viral vector such as an adeno-associated viral (AAV) vector) can be designed such that it can insert into a location within a cbl-b gene to disrupt the cbl-b gene in the genetically engineered T cells and express the CAR polypeptide. Disruption of cbl-b leads to loss of function of the endogenous cbl-b protein. For example, a disruption in the cbl-b gene can be created with an endonuclease such as those described herein and one or more gRNAs targeting one or more cbl-b genomic regions. Any of the gRNAs specific to a cbl-b gene and the target regions disclosed herein can be used for this purpose.

In some examples, a genomic deletion in the cbl-b gene and replacement by a CAR coding segment can be created by homology directed repair or HDR (e.g., using a donor template, which may be part of a viral vector such as an adeno-associated viral (AAV) vector). In some embodiments, a disruption in the cbl-b gene can be created with an endonuclease as those disclosed herein and one or more gRNAs targeting one or more cbl-b genomic regions and inserting a CAR coding segment into the cbl-b gene.

In some embodiments, a nucleic acid encoding a CAR (e.g., via a donor template, which can be carried by a viral vector such as an adeno-associated viral (AAV) vector) can be designed such that it can insert into a location within a CD70 gene to disrupt the CD70 gene in the genetically engineered T cells and express the CAR polypeptide. Disruption of cbl-b leads to loss of function of the endogenous CD70 receptor. For example, a disruption in the CD70 gene can be created with an endonuclease such as those described herein and one or more gRNAs targeting one or more CD70 genomic regions. Any of the gRNAs specific to a CD70 gene and the target regions disclosed herein can be used for this purpose.

In some examples, a genomic deletion in the CD70 gene and replacement by a CAR coding segment can be created by homology directed repair or HDR (e.g., using a donor template, which may be part of a viral vector such as an adeno-associated viral (AAV) vector). In some embodiments, a disruption in the CD70 gene can be created with an endonuclease as those disclosed herein and one or more gRNAs targeting one or more CD70 genomic regions and inserting a CAR coding segment into the CD70 gene.

In some embodiments, a nucleic acid encoding a CAR (e.g., via a donor template, which can be carried by a viral vector such as an adeno-associated viral (AAV) vector) can be designed such that it can insert into a location within a Reg1 gene to disrupt the Reg1 gene in the genetically engineered T cells and express the CAR polypeptide. Disruption of Reg1 leads to loss of function of the endogenous Reg1 protein. For example, a disruption in the Reg1 gene can be created with an endonuclease such as those described herein and one or more gRNAs targeting one or more Reg1 genomic regions. Any of the gRNAs specific to a Reg1 gene and the target regions disclosed herein can be used for this purpose.

In some examples, a genomic deletion in the Reg1 gene and replacement by a CAR coding segment can be created by homology directed repair or HDR (e.g., using a donor template, which may be part of a viral vector such as an adeno-associated viral (AAV) vector). In some embodiments, a disruption in the Reg1 gene can be created with an endonuclease as those disclosed herein and one or more gRNAs targeting one or more Reg1 genomic regions, and inserting a CAR coding segment into the Reg1 gene.

In some embodiments, a nucleic acid encoding a CAR (e.g., via a donor template, which can be carried by a viral vector such as an adeno-associated viral (AAV) vector) can be designed such that it can insert into a location within a TGFBRII gene to disrupt the TGFBRII gene in the genetically engineered T cells and express the CAR polypeptide. Disruption of Reg1 leads to loss of function of the endogenous TGFBRII receptor. For example, a disruption in the TGFBRII gene can be created with an endonuclease such as those described herein and one or more gRNAs targeting one or more TGFBRII genomic regions. Any of the gRNAs specific to a TGFBRII gene and the target regions disclosed herein can be used for this purpose.

In some examples, a genomic deletion in the TGFBRII gene and replacement by a CAR coding segment can be created by homology directed repair or HDR (e.g., using a donor template, which may be part of a viral vector such as an adeno-associated viral (AAV) vector). In some embodiments, a disruption in the TGFBRII gene can be created with an endonuclease as those disclosed herein and one or more gRNAs targeting one or more TGFBRII genomic regions, and inserting a CAR coding segment into the TGFBRII gene.

A donor template as disclosed herein can contain a coding sequence for a CAR. In some examples, the CAR-coding sequence may be flanked by two regions of homology to allow for efficient HDR at a genomic location of interest, for example, at a TRAC gene using a gene editing method known in the art. In some examples, a CRISPR-based method can be used. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous prompter (e.g., downstream, or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter, see, e.g., SEQ ID NO: 238 provided in Table 13 below. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

When needed, additional gene editing (e.g., gene knock-in or knock-out) can be introduced into therapeutic T cells as disclosed herein to improve T cell function and therapeutic efficacy. For example, if β2M disruption can be performed to reduce the risk of or prevent a host-versus-graft response. Other examples include knock-in or knock-out genes to improve target cell lysis, knock-in or knock-out genes to enhance performance of therapeutic T cells such as CAR-T cells.

In some embodiments, a donor template for delivering an anti-BCMA CAR may be an AAV vector inserted with a nucleic acid fragment comprising the coding sequence of the anti-BCMA CAR, and optionally regulatory sequences for expression of the anti- BCMA CAR (e.g., a promoter such as the EF1a promoter provided in the sequence Table 13), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 14. In some specific examples, the donor template for delivering the anti- BCMA CAR may comprise a nucleotide sequence of SEQ ID NO: 273, which can be inserted into a disrupted TRAC gene, for example, replacing the fragment of SEQ ID NO: 14.

In some embodiments, a donor template for delivering an anti-CD70 CAR may be an AAV vector inserted with a nucleic acid fragment comprising the coding sequence of the anti-CD70 CAR, and optionally regulatory sequences for expression of the anti-CD70 CAR (e.g., a promoter such as the EF1α promoter provided in the Table 13), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 14. In some specific examples, the donor template for delivering the anti-CD70 CAR may comprise a nucleotide sequence of SEQ ID NO: 267, which can be inserted into a disrupted TRAC gene, for example, replacing the fragment of SEQ ID NO: 14.

The genetically engineered T cells having a disrupted cbl-b gene, additional disrupted genes, e.g., β2M, TRAC, CD70, Reg1, and/or TGFBRII, and further expressing a chimeric antigen receptor (CAR) can be produced by sequential targeting of the genes of interest. For example, in some embodiments, the cbl-b gene may be disrupted first, followed by disruption of TRAC and β2M genes and CAR insertion. In other embodiments, TRAC and β2M genes may be disrupted first, followed by CAR insertion and disruption of the cbl-b gene. Accordingly, in some embodiments, the genetically engineered T cells disclosed herein may be produced by multiple, sequential electroporation events with multiple RNPs targeting the genes of interest, e.g., cbl-b, β2M, TRAC, CD70, Reg1, and TGFBRII., etc.

In other embodiments, the genetically engineered CAR T cells disclosed herein may be produced by a single electroporation event with an RNP complex comprising an RNA-guided nuclease and multiple gRNAs targeting the genes of interest, e.g., cbl-b, β2M, TRAC, CD70, Reg1, and TGFBRII. etc.

### (C) Exemplary Genetically Engineered T Cells Expression a Chimeric Antigen Receptor

It should be understood that gene disruption encompasses gene modification through gene editing (e.g., using CRISPR/Cas gene editing to insert or delete one or more nucleotides). A disrupted gene may contain one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In some embodiments, a population of genetically engineered T cells disclosed herein express a CAR (e.g., anti-BCMA, or anti-CD70 CAR), a disrupted cbl-b gene, and optionally a disrupted CD70 gene, a disrupted TRAC gene, a disrupted β2M gene, a disrupted Reg1 gene, a disrupted TGFBRII gene, or a combination thereof. The nucleotide sequence encoding the CAR may be inserted in the disrupted TRAC gene (e.g., replacing the site targeted by a sgRNA such as TA-1; see Table 1). In some examples, such a population of genetically engineered T cells may comprise about 70-99% cbl-b⁻ cells, for example about 90-97% cbl-b⁻ cells, for example about 80-89% CD70⁻ cells, about 70-99% TCR⁻ cells, for example about 90-99% TCR⁻ cells, and/or optionally about 60-99% β2M⁻ cells, for example about 60-82% β2M⁻ cells, and/or optionally about 70-99% CD70⁻ cells, for example about 90-99% CD70⁻ cells. In some examples, such a population of genetically engineered T cells may comprise about 70-99% Reg1⁻ cells, for example about 90-97% Reg1⁻ cells, and/or about 70-99% TGFBRII⁻ cells, e.g., for example about 80-89% TGFBRII⁻ cells. The cell population may also contain at least about 30%-50% (e.g., at least 60%) cells expressing the CAR.

In some examples, the genetically engineered T cells disclosed herein comprise a disrupted cbl-b gene, which may comprise one or more of the modified sequences provided in Tables 3-11.

### (I) Anti-BCMA CAR-T Cells Having Cbl-b Disruption

Also provided herein is population of genetically engineered immune cells (e.g., T cells such as human T cells) comprising a disrupted cbl-b gene and expressing an anti-BCMA CAR, e.g., those disclosed herein. In some examples, the anti-BCMA CAR T cells disclosed herein, which express any of the anti-BCMA CAR disclosed herein (e.g., the anti-BCMA CAR comprising the amino acid sequence of SEQ ID NO: 274), may also comprise a disrupted TRAC gene, a disrupted β2M gene, a disrupted CD70 gene, a disrupted Reg1 gene, and/or a disrupted TGFBRII gene as also disclosed herein.

In some examples, the genetically engineered anti-BCMA CAR T cells disclosed herein that comprise a disrupted cbl-b gene, which may comprise one or more of the modified sequences provided in Tables 3-11.

In some instances, the population of genetically engineered immune cells (e.g., T cells such as human T cells) comprising both a disrupted cbl-b gene and a disrupted CD70 gene, and expressing an anti-BCMA CAR, e.g., those disclosed herein. In some examples anti-BCMA CAR T cells are anti-BCMA CAR T cells having disrupted TRAC gene and β2M gene. The nucleic acid encoding the anti-BCMA CAR can be inserted in the disrupted TRAC gene at the site of SEQ ID NO: 14, which, in some instances, can be replaced by the nucleic acid encoding the anti-BCMA CAR, thereby disrupting expression of the TRAC gene. The disrupted TRAC gene in the anti-BCMA CAR T cells may comprise the nucleotide sequence of SEQ ID NO: 273. In some examples, the anti-BCMA CAR-T cells may further comprise a disrupted Reg1 gene and/or a disrupted TGFBRII gene.

Anti-BCMA CAR T cells that comprise a disrupted cbl-b gene can be produced via ex vivo genetic modification using the CRISPR/Cas9 (Clustered Regularly Interspaced Short Palindromic Repeats/CRISPR associated protein 9) technology to disrupt targeted genes (cbl-b, and optionally TRAC and β2M genes), and adeno-associated virus (AAV) transduction to deliver the anti-BCMA CAR construct. CRISPR-Cas9-mediated gene editing involves at least three guide RNAs (sgRNAs).

Anti-BCMA CAR T cells that comprise a disrupted cbl-b gene, and optionally the additional gene edits, can be produced via ex vivo genetic modification using the CRISPR/Cas9 (Clustered Regularly Interspaced Short Palindromic Repeats/CRISPR associated protein 9) technology to disrupt targeted genes (cbl-b, and optionally CD70, TRAC, β2M, Reg1, and/or TGFBRII genes), and adeno-associated virus (AAV) transduction to deliver the anti-BCMA CAR construct. CRISPR-Cas9-mediated gene editing involves at least three guide RNAs (sgRNAs), as described above for anti-BCMA CAR T cells.

Anti-BCMA CAR T cells that comprise a disrupted cbl-b gene and optionally one or more of the additional gene edits as disclosed herein can be produced via ex vivo genetic modification using the CRISPR/Cas9 (Clustered Regularly Interspaced Short Palindromic Repeats/CRISPR associated protein 9) technology to disrupt targeted genes (cbl-b, and optionally CD70, TRAC, β2M, Reg1, and/or TGFBRII genes), and adeno-associated virus (AAV) transduction to deliver the anti-BCMA CAR construct. CRISPR-Cas9-mediated gene editing involves at least three guide RNAs (sgRNAs), as described above for anti-BCMA CAR T cells.

Anti-BCMA CAR T cells that comprise a disrupted Reg1 gene can be produced via ex vivo genetic modification using the CRISPR/Cas9 (Clustered Regularly Interspaced Short Palindromic Repeats/CRISPR associated protein 9) technology to disrupt targeted genes (Reg1, and optionally TRAC, β2M, CD70 and/or TGFBRII genes), and adeno-associated virus (AAV) transduction to deliver the anti-CD70 CAR construct. CRISPR-Cas9-mediated gene editing involves at least an sgRNA targeting the Reg 1 gene as those disclosed herein (see, e.g., Table 20 such as SEQ ID NO: 337), and optionally an sgRNA (SEQ ID NO: 11) which targets the CD70 locus, TA-1 sgRNA (SEQ ID NO: 3) which targets the TRAC locus, and β2M-1 sgRNA (SEQ ID NO: 7) which targets the β2M locus.

Anti-BCMA CAR T cells that comprise a disrupted TGFBRII gene can be produced via ex vivo genetic modification using the CRISPR/Cas9 (Clustered Regularly Interspaced Short Palindromic Repeats/CRISPR associated protein 9) technology to disrupt targeted genes (TGFBRII, and optionally, TRAC, β2M, CD70, and/or Reg1 genes), and adeno-associated virus (AAV) transduction to deliver the anti-CD70 CAR construct. CRISPR-Cas9-mediated gene editing involves at least an sgRNA targeting the TGFBRII gene as those disclosed herein (see, e.g., Table 21, such as SEQ ID NO: 393), and optionally an sgRNA (SEQ ID NO: 11) which targets the CD70 locus, TA-1 sgRNA (SEQ ID NO: 3) which targets the TRAC locus, and β2M-1 sgRNA (SEQ ID NO: 7).

Anti-BCMA CAR T cells that comprise a disrupted TGFBRII gene and a disrupted Reg1 gene can be produced via ex vivo genetic modification using the CRISPR/Cas9 (Clustered Regularly Interspaced Short Palindromic Repeats/CRISPR associated protein 9) technology to disrupt targeted genes (TGFBRII and Reg1, and optionally, TRAC, β2M, and/or CD70 genes), and adeno-associated virus (AAV) transduction to deliver the anti-CD70 CAR construct. CRISPR-Cas9-mediated gene editing involves at least an sgRNA targeting the TGFBRII gene as those disclosed herein (see, e.g., Table 21, such as SEQ ID NO: 393), and an sgRNA targeting the Reg1 gene as those disclosed herein (see, e.g., Table 20, such as SEQ ID NO: 337), and optionally an sgRNA (SEQ ID NO: 11) which targets the CD70 locus, TA-1 sgRNA (SEQ ID NO: 3) which targets the TRAC locus, and β2M-1 sgRNA (SEQ ID NO: 7) which targets the β2M locus.

The anti-BCMA CAR T cells are composed of an anti-BCMA single-chain antibody fragment (scFv, which may comprise the amino acid sequence of SEQ ID NO: 277), followed by a CD8 hinge and transmembrane domain (e.g., comprising the amino acid sequence of SEQ ID NO: 243) that is fused to an intracellular co-signaling domain of CD28 (e.g., SEQ ID NO: 247) and a CD3ζ signaling domain (e.g., SEQ ID NO: 249). In specific examples, the anti-BCMA CAR T cells comprise the amino acid sequence of SEQ ID NO: 275.

In some embodiments, at least 30% of a population of anti-BCMA CAR T cells express a detectable level of the anti-BCMA CAR. For example, at least 40%, at least 50%, at least 60%, at least 70%, at least 75%, at least 80%, at least 85%, at least 90%, or at least 95% of the anti-BCMA CAR T cells express a detectable level of the anti-BCMA CAR.

In some examples, the anti-BCMA CAR T cells may comprise at least 80% cbl-b⁻ cells, for example, at least 85%, at least 90%, at least 95%, at least 98% or above cbl-b⁻ cells.

In some embodiments, at least 50% of a population of anti-BCMA CAR T cells may not express a detectable level of β2M surface protein. For example, at least 55%, at least 60%, at least 70%, at least 75%, at least 80%, at least 85%, at least 90%, or at least 95% of the anti-BCMA CAR T cells may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively, or in addition, at least 50% of a population of anti-BCMA CAR T cells may not express a detectable level of TRAC surface protein. For example, at least 55%, at least 60%, at least 70%, at least 75%, at least 80%, at least 85%, at least 90%, or at least 95% of the anti-BCMA CAR T cells may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein. In specific examples, more than 90% (e.g., more than 99.5%) of the anti-BCMA CAR T cells do not express a detectable TRAC surface protein.

In some embodiments, a substantial percentage of the population of anti-BCMA CAR T cells may comprise more than one gene edit, which results in a certain percentage of cells not expressing more than one gene and/or protein.

For example, at least 50% of a population of anti-BCMA CAR T cells may not express a detectable level of two surface proteins, e.g., does not express a detectable level of β2M and TRAC proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the anti-BCMA CAR T cells do not express a detectable level of TRAC and β2M surface proteins. In another example, at least 50% of a population of anti-BCMA CAR T cells do not express a detectable level of TRAC and β2M surface proteins.

See also WO 2019/097305A2, and WO2019215500, the relevant disclosures of each of which are incorporated by reference for the subject matter and purpose referenced herein.

In specific examples, the genetically engineered T cell population may be the anti-BCMA CAR T cells disclosed herein that further comprise a disrupted CD70 gene. In some examples, the disrupted CD70 gene may comprise a nucleotide sequence selected from those listed in Table 1 below. In some examples, the anti-BCMA CAR T cells may comprise at least 80% CD70⁻ cells, for example, at least 85%, at least 90%, at least 95%, at least 98% or above CD70⁻ cells.

In specific examples, the genetically engineered T cell population may be the anti-BCMA CAR T cells disclosed herein that further comprise a disrupted CD70 gene and a disrupted cbl-b gene. The disrupted cbl-b gene may comprise any of the sequences provided in Tables 3-11 below. Alternatively, or in addition, the disrupted CD70 gene may comprise a nucleotide sequence selected from those listed in Table 1 below. In some examples, the anti-BCMA CAR T cells may comprise at least 80% CD70⁻ cells, for example, at least 85%, at least 90%, at least 95%, at least 98% or above CD70 ⁻ cells. Alternatively, or in addition, the anti-BCMA CAR T cells may comprise at least 80% cbl-b⁻ cells, for example, at least 85%, at least 90%, at least 95%, at least 98% or above cbl-b⁻ cells. In some examples, the anti-BCMA CAR T cells may comprise at least 60% cbl-b⁻/CD70⁻ cells, for example, at least 65%, at least 70%, at least 75%, at least 80%, at least 85%, at least 90%, at least 95%, at least 98% or above cbl-b⁻ /CD70⁻ cells.

In specific examples, the genetically engineered T cell population may be the anti-BCMA CAR T cells disclosed herein that further comprise a disrupted Reg1 gene. In some examples, the anti-BCMA CAR T cells may comprise at least 80% Reg1⁻ cells, for example, at least 85%, at least 90%, at least 95%, at least 98% or above Reg1⁻ cells.

In specific examples, the genetically engineered T cell population may be the anti-BCMA CAR T cells disclosed herein that further comprise a disrupted TGFBRII gene. In some examples, the anti-BCMA CAR T cells may comprise at least 80% TGFBRII⁻ cells, for example, at least 85%, at least 90%, at least 95%, at least 98% or above TGFBRII ⁻ cells.

In specific examples, the genetically engineered T cell population may be the anti-BCMA CAR T cells disclosed herein that further comprise a disrupted TGFBRII gene and a disrupted Reg1 gene. In some examples, the anti-BCMA CAR T cells may comprise at least 80% TGFBRII⁻ cells, for example, at least 85%, at least 90%, at least 95%, at least 98% or above TGFBRII ⁻ cells. Alternatively or in addition, the anti-BCMA CAR T cells may comprise at least 80% Reg1⁻ cells, for example, at least 85%, at least 90%, at least 95%, at least 98% or above Reg ⁻ cells. In some examples, the anti-BCMA CAR T cells may comprise at least 60% Reg1⁻/TGFBRII⁻ cells, for example, at least 65%, at least 70%, at least 75%, at least 80%, at least 85%, at least 90%, at least 95%, at least 98% or above Reg1⁻/TGFBRII⁻ cells.

### (II). Anti-CD70 CAR-T Cells Having Cbl-b And/or CD70 Gene Disruption

Also provided herein is population of genetically engineered immune cells (e.g., T cells such as human T cells) comprising a disrupted cbl-b gene, and optionally one or more of a disrupted CD70 gene, a disrupted TRAC gene, a disrupted β2M gene, a disrupted Reg1 gene, a disrupted TGFBRII gene, and expressing anti-CD70 CAR, e.g., those disclosed herein. In some examples, the anti-CD70 CAR T cells disclosed herein, which express any of the anti-CD70 CAR disclosed herein (e.g., the anti-CD70 CAR comprising the amino acid sequence of SEQ ID NO: 265), may also comprise a disrupted TRAC gene, a disrupted β2M gene, a disrupted CD70 gene, a disrupted Reg1 gene, and/or a disrupted TGFBRII gene as also disclosed herein.

In some examples, the genetically engineered anti-CD70 CAR T cells disclosed herein that comprise a disrupted cbl-b gene, which may comprise one or more of the modified sequences provided in Tables 3-11.

In some examples anti-CD70 CAR T cells are anti-CD70 CAR T cells having disrupted TRAC gene, a disrupted β2M gene, a disrupted CD70 gene, a disrupted Reg1 gene, and a disrupted TGFBRII gene. The nucleic acid encoding the anti-CD70 CAR can be inserted in the disrupted TRAC gene at the site of SEQ ID NO: 14, which is replaced by the nucleic acid encoding the anti-CD70 CAR, thereby disrupting expression of the TRAC gene. The disrupted TRAC gene in the anti-CD70 CAR T cells may comprise the nucleotide sequence of SEQ ID NO: 267.

Anti-CD70 CAR T cells that comprise a disrupted cbl-b gene can be produced via ex vivo genetic modification using the CRISPR/Cas9 (Clustered Regularly Interspaced Short Palindromic Repeats/CRISPR associated protein 9) technology to disrupt targeted genes (cbl-b, and optionally TRAC, β2M and/or CD70 genes), and adeno-associated virus (AAV) transduction to deliver the anti-CD70 CAR construct. CRISPR-Cas9-mediated gene editing involves at least an sgRNA targeting the cbl-b gene as those disclosed herein (see, e.g., Table 2), and optionally an sgRNA (SEQ ID NO: 9) which targets the CD70 locus, TA-1 sgRNA (SEQ ID NO: 1) which targets the TRAC locus, and β2M-1 sgRNA (SEQ ID NO: 5), which targets the β2M locus.

Anti-CD70 CAR T cells that comprise a disrupted CD70 gene can be produced via ex vivo genetic modification using the CRISPR/Cas9 (Clustered Regularly Interspaced Short Palindromic Repeats/CRISPR associated protein 9) technology to disrupt targeted genes (CD70, and optionally, TRAC, β2M, and/or CD70 genes), and adeno-associated virus (AAV) transduction to deliver the anti-CD70 CAR construct. CRISPR-Cas9-mediated gene editing involves at least an sgRNA targeting the CD70 gene as those disclosed herein (see, e.g., Table 1), and optionally an sgRNA (SEQ ID NO: 9) which targets the CD70 locus, TA-1 sgRNA (SEQ ID NO: 1) which targets the TRAC locus, and β2M-1 sgRNA (SEQ ID NO: 5) which targets the β2M locus.

Anti-CD70 CAR T cells that comprise a disrupted Reg1 gene can be produced via ex vivo genetic modification using the CRISPR/Cas9 (Clustered Regularly Interspaced Short Palindromic Repeats/CRISPR associated protein 9) technology to disrupt targeted genes (Reg1, and optionally TRAC, β2M, CD70 and/or TGFBRII genes), and adeno-associated virus (AAV) transduction to deliver the anti-CD70 CAR construct. CRISPR-Cas9-mediated gene editing involves at least an sgRNA targeting the Reg 1 gene as those disclosed herein (see, e.g., Table 20 such as SEQ ID NO: 337), and optionally an sgRNA (SEQ ID NO: 11) which targets the CD70 locus, TA-1 sgRNA (SEQ ID NO: 3) which targets the TRAC locus, and β2M-1 sgRNA (SEQ ID NO: 7) which targets the β2M locus.

Anti-CD70 CAR T cells that comprise a disrupted TGFBRII gene can be produced via ex vivo genetic modification using the CRISPR/Cas9 (Clustered Regularly Interspaced Short Palindromic Repeats/CRISPR associated protein 9) technology to disrupt targeted genes (TGFBRII, and optionally, TRAC, β2M, CD70, and/or Reg1 genes), and adeno-associated virus (AAV) transduction to deliver the anti-CD70 CAR construct. CRISPR-Cas9-mediated gene editing involves at least an sgRNA targeting the TGFBRII gene as those disclosed herein (see, e.g., Table 21, such as SEQ ID NO: 393), and optionally an sgRNA (SEQ ID NO: 11) which targets the CD70 locus, TA-1 sgRNA (SEQ ID NO: 3) which targets the TRAC locus, and β2M-1 sgRNA (SEQ ID NO: 7) which targets the β2M locus.

Anti-CD70 CAR T cells that comprise a disrupted TGFBRII gene and a disrupted Reg1 gene can be produced via ex vivo genetic modification using the CRISPR/Cas9 (Clustered Regularly Interspaced Short Palindromic Repeats/CRISPR associated protein 9) technology to disrupt targeted genes (TGFBRII and Reg1, and optionally, TRAC, β2M, and/or CD70 genes), and adeno-associated virus (AAV) transduction to deliver the anti-CD70 CAR construct. CRISPR-Cas9-mediated gene editing involves at least an sgRNA targeting the TGFBRII gene as those disclosed herein (see, e.g., Table 21, such as SEQ ID NO: 393), and an sgRNA targeting the Reg1 gene as those disclosed herein (see, e.g., Table 20, such as SEQ ID NO: 337), and optionally an sgRNA (SEQ ID NO: 11) which targets the CD70 locus, TA-1 sgRNA (SEQ ID NO: 3) which targets the TRAC locus, and β2M-1 sgRNA (SEQ ID NO: 7) which targets the β2M locus.

The anti-CD70 CAR T cells are composed of an anti-CD70 CAR single-chain antibody fragment (scFv, which may comprise the amino acid sequence of SEQ ID NO: 265), followed by a CD8 hinge and transmembrane domain (e.g., comprising the amino acid sequence of SEQ ID NO: 243) that is fused to an intracellular co-signaling domain of CD28 (e.g., SEQ ID NO: 247) and a CD3ζ signaling domain (e.g., SEQ ID NO: 249). In specific examples, the anti-CD70 CAR T cells comprise the amino acid sequence of SEQ ID NO: 265.

In some embodiments, at least 30% of a population of anti-CD70 CAR T cells express a detectable level of the anti-CD70 CAR. For example, at least 40%, at least 50%, at least 60%, at least 70%, at least 75%, at least 80%, at least 85%, at least 90%, or at least 95% of the anti-CD70 CAR T cells express a detectable level of the anti-CD70 CAR.

In some examples, the anti-CD70 CAR T cells may comprise at least 80% cbl-b⁻ cells, for example, at least 85%, at least 90%, at least 95%, at least 98% or above cbl-b⁻ cells.

In some embodiments, at least 50% of a population of anti-CD70 CAR T cells may not express a detectable level of β2M surface protein. For example, at least 55%, at least 60%, at least 70%, at least 75%, at least 80%, at least 85%, at least 90%, or at least 95% of the anti-CD70 CAR T cells may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively, or in addition, at least 50% of a population of anti-CD70 CAR T cells may not express a detectable level of TRAC surface protein. For example, at least 55%, at least 60%, at least 70%, at least 75%, at least 80%, at least 85%, at least 90%, or at least 95% of the anti-CD70 CAR T cells may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein. In specific examples, more than 90% (e.g., more than 99.5%) of the anti-CD70 CAR T cells do not express a detectable TRAC surface protein.

In some embodiments, at least 50% of a population of the anti-CD70 CAR T cells may not express a detectable level of CD70 surface protein. For example, at least 55%, at least 60%, at least 70%, at least 75%, at least 80%, at least 85%, at least 90%, at least 95%, or at least 98% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, or 95%-100% of the engineered T cells of a population does not express a detectable level of CD70 surface protein.

In some embodiments, a substantial percentage of the population of anti-CD70 CAR T cells may comprise more than one gene edit, which results in a certain percentage of cells not expressing more than one gene and/or protein.

For example, at least 50% of a population of anti-CD70 CAR T cells may not express a detectable level of two surface proteins, e.g., does not express a detectable level of β2M and TRAC proteins, β2M and CD70 proteins, or TRAC and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of two surface proteins. In another example, at least 50% of a population of the Anti-CD70 CAR cells may not express a detectable level of all of the three target surface proteins β2M, TRAC, and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M, TRAC, and CD70 surface proteins.

In some embodiments, the population of anti-CD70 CAR T cells may comprise more than one gene edit (e.g., in more than one gene), which may be an edit described herein. For example, the population of anti-CD70 CAR T cells may comprise a disrupted TRAC gene via the CRISPR/Cas technology using the TA-1 TRAC gRNA. In some examples, the anti-CD70 CAR T cells may comprise a deletion in the TRAC gene relative to unmodified T cells. For example, the anti-CD70 CAR T cells may comprise a deletion of the fragment AGAGCAACAGTGCTGTGGCC (SEQ ID NO: 14) in the TRAC gene. This fragment can be replaced by the nucleic acid encoding the anti-CD70 CAR (e.g., SEQ ID NO: 267). Alternatively, or in addition, the population of anti-CD70 CAR T cells may comprise a disrupted β2M gene via CRISPR/Cas9 technology using the gRNA of β2M-1. In specific examples, anti-CD70 CAR T cells comprise ≥ 30% CAR⁺ T cells, ≤ 50% β2M⁺ cells, and ≤ 30% TCRαβ⁺ cells. In additional specific examples, anti-CD70 CAR T cells comprise ≥ 30% CAR⁺ T cells, ≤ 30% β2M⁺ cells, and ≤ 0.5% TCRαβ⁺ cells.

See also WO 2019/097305A2, and WO2019215500, the relevant disclosures of each of which are incorporated by reference for the subject matter and purpose referenced herein.

In specific examples, the genetically engineered T cell population may be the anti-CD70 CAR T cells disclosed herein that further comprise a disrupted cbl-b gene. The disrupted cbl-b gene may comprise any of the sequences provided in Tables 3-11 below. Such a genetically engineered T cells may have ≥ 30% CAR+ T cells, ≤ 0.4% TCR⁺ T cells, ≤ 30% β2M⁺ T cells, and ≤ 2% CD70⁺ T cells. In some examples, the anti-CD70 CAR T cells may comprise at least 80% cbl-b⁻ cells, for example, at least 85%, at least 90%, at least 95%, at least 98% or above cbl-b⁻ cells.

In specific examples, the genetically engineered T cell population may be the anti-CD70 CAR T cells disclosed herein that further comprise a disrupted CD70 gene. Such a genetically engineered T cells may have ≥ 30% CAR+ T cells, ≤ 0.4% TCR⁺ T cells, ≤ 30% β2M⁺ T cells, and ≤ 2% CD70⁺ T cells. In some examples, the anti-CD70 CAR T cells may comprise at least 80% CD70⁻ cells, for example, at least 85%, at least 90%, at least 95%, at least 98% or above CD70⁻ cells.

In specific examples, the genetically engineered T cell population may be the anti-CD70 CAR T cells disclosed herein that further comprise a disrupted Reg1 gene. Such a genetically engineered T cells may have ≥ 30% CAR+ T cells, ≤ 0.4% TCR⁺ T cells, ≤ 30% β2M⁺ T cells, and ≤ 2% CD70⁺ T cells. In some examples, the anti-CD70 CAR T cells may comprise at least 80% Reg1⁻ cells, for example, at least 85%, at least 90%, at least 95%, at least 98% or above Regl⁻ cells.

In specific examples, the genetically engineered T cell population may be the anti-CD70 CAR T cells disclosed herein that further comprise a disrupted TGFBRII gene. Such a genetically engineered T cells may have ≥ 30% CAR+ T cells, ≤ 0.4% TCR⁺ T cells, ≤ 30% β2M⁺ T cells, and ≤ 2% CD70⁺ T cells. In some examples, the anti-CD70 CAR T cells may comprise at least 80% TGFBRII⁻ cells, for example, at least 85%, at least 90%, at least 95%, at least 98% or above TGFBRII⁻ cells.

In specific examples, the genetically engineered T cell population may be the anti-CD70 CAR T cells disclosed herein that further comprise a disrupted TGFBRII gene and a disrupted Reg1 gene. Such a genetically engineered T cells may have ≥ 30% CAR+ T cells, ≤ 0.4% TCR⁺ T cells, ≤ 30% β2M⁺ T cells, and ≤ 2% CD70⁺ T cells. In some examples, the anti-CD70 CAR T cells may comprise at least 80% TGFBRII⁻ cells, for example, at least 85%, at least 90%, at least 95%, at least 98% or above TGFBRII⁻ cells. In some examples, the anti-CD70 CAR T cells may comprise at least 60% Reg1⁻/TGFBRII⁻ cells, for example, at least 65%, at least 70%, at least 75%, at least 80%, at least 85%, at least 90%, at least 95%, at least 98% or above Reg1⁻/TGFBRII⁻cells.

### III. Therapeutic Applications

The therapeutic T cells generated using the genetically engineered T cells disclosed herein would be expected to maintain T cell health enabled by the disruption of the cbl-b gene, and optionally the disruption of the CD70 gene, the disruption of the TRAC gene, the disruption of the β2M gene, the disrupted Reg1 gene, the disrupted TGFBRII gene, or a combination thereof. For example, maintaining T cell health may extend expansion during manufacturing, thereby increasing yield and consistency. In another example, maintaining T cell health may rescue exhausted/unhealthy T cells, thereby enabling potentially lower doses in patients and more robust responses.

The therapeutic T cells disclosed herein can be administered to a subject for therapeutic purposes, for example, treatment of a solid tumor targeted by the CAR construct expressed by the therapeutic T cells.

The step of administering may include the placement (e.g., transplantation) of the therapeutic T cells into a subject by a method or route that results in at least partial localization of the therapeutic T cells at a desired site, such as a tumor site, such that a desired effect(s) can be produced. Therapeutic T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the lifetime of the subject, i.e., long-term engraftment. For example, in some aspects described herein, an effective amount of the therapeutic T cells can be administered via a systemic route of administration, such as an intraperitoneal or intravenous route.

In some embodiments, the therapeutic T cells are administered systemically, which refers to the administration of a population of cells other than directly into a target site, tissue, or organ, such that it enters, instead, the subject’s circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

A subject may be any subject for whom diagnosis, treatment, or therapy is desired. In some embodiments, the subject is a mammal. In some embodiments, the subject is a human.

In some instances, the therapeutic T cells may be autologous (“self”) to the subject, i.e., the cells are from the same subject. Alternatively, the therapeutic T cells can be non-autologous (“non-self,” e.g., allogeneic, syngeneic or xenogeneic) to the subject. “Allogeneic” means that the therapeutic T cells are not derived from the subject who receives the treatment but from different individuals (donors) of the same species as the subject. A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used.

In some embodiments, an engineered T cell population being administered according to the methods described herein comprises allogeneic T cells obtained from one or more donors. Allogeneic refers to a cell, cell population, or biological samples comprising cells, obtained from one or more different donors of the same species, where the genes at one or more loci are not identical to the recipient (e.g., subject). For example, an engineered T cell population, being administered to a subject can be derived from one or more unrelated donors, or from one or more non-identical siblings. In some embodiments, syngeneic cell populations may be used, such as those obtained from genetically identical donors, (e.g., identical twins). In some embodiments, the cells are autologous cells; that is, the engineered T cells are obtained or isolated from a subject and administered to the same subject, i.e., the donor and recipient are the same.

An effective amount refers to the amount of a population of engineered T cells needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

Because of the enhanced persistence and efficacy of the therapeutic T cells disclosed herein, the dose of the therapeutic T cells provided herein would be lower than the standard dose of CAR-T cells prepared by conventional approaches (e.g., using T cells that do not have one or more of the genetic editing events disclosed herein, including a disrupted cbl-b gene and optionally one or more of the additional gene edits, e.g., a disrupted CD70 gene, a disrupted TRAC gene, a disrupted β2M gene, a disrupted Reg1 gene, and/or a disrupted TGFBRII gene). In some examples, the effective amount of the therapeutic T cells disclosed herein may be at least 2-fold lower, at least 5-fold lower, at least 10-fold lower, at least 20-fold lower, at least 50-fold lower, or at least 100-fold lower than a standard dose of a CAR-T therapy. In some examples, an effective amount of the therapeutic T cells disclosed herein may be less than 10⁶ cells, e.g., 10⁵ cells, 5 ×10⁴ cells, 10⁴ cells, 5× 10³ cells, or 10³ cells. In some examples described herein, the cells are expanded in culture prior to administration to a subject in need thereof.

The efficacy of a treatment using the therapeutic T cells disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

Combination therapies are also encompassed by the present disclosure. For example, the therapeutic T cells disclosed herein may be co-used with other therapeutic agents, for treating the same indication, or for enhancing efficacy of the therapeutic T cells and/or reducing side effects of the therapeutic T cells.

### IV. Kits

The present disclosure also provides kits for use in producing the genetically engineered T cells, the therapeutic T cells, and for therapeutic uses,

In some embodiments, a kit provided herein may comprise components for performing genetic edit of cbl-b gene, and one or more additional gene edits (e.g., disrupting the CD70 gene, the TRAC gene, the β2M gene, the Reg1 gene, the TGFBRII gene, or a combination thereof) and optionally a population of immune cells to which the genetic editing will be performed (e.g., a leukopak). A leukopak sample may be an enriched leukapheresis product collected from peripheral blood. It typically contains a variety of blood cells including monocytes, lymphocytes, platelets, plasma, and red cells. The components for genetically editing one or more of the target genes may comprise a suitable endonuclease such as an RNA-guided endonuclease and one or more nucleic acid guides, which direct cleavage of one or more suitable genomic sites by the endonuclease. For example, the kit may comprise a Cas enzyme such as Cas 9 and one or more gRNAs targeting a cbl-b gene. Any of the gRNAs specific to these target genes can be included in the kit. Such a kit may further comprise components for further gene editing, for example, gRNAs and optionally additional endonucleases for editing other target genes such as CD70, β2M, TRAC, Reg1, and/or TGFBRII.

In some embodiments, a kit provided herein may comprise a population of genetically engineered T cells as disclosed herein, and one or more components for producing the therapeutic T cells as also disclosed herein. Such components may comprise an endonuclease suitable for gene editing and a nucleic acid coding for a CAR construct of interest. The CAR-coding nucleic acid may be part of a donor template as disclosed herein, which may contain homologous arms flanking the CAR-coding sequence. In some instances, the donor template may be carried by a viral vector such as an AAV vector.

The kit may further comprise gRNAs specific to a TRAC gene for inserting the CAR-coding sequence into the TRAC gene. In other examples, the kit may further comprise gRNAs specific to a β2M gene for inserting the CAR-coding sequence into the β2M gene. In other examples, the kit may further comprise gRNAs specific to a CD70 gene for inserting the CAR-coding sequence into the CD70 gene. In yet other examples, the kit may further comprise gRNAs specific to a cbl-b gene for inserting the CAR-coding sequence into the cbl-b gene. In still other examples, the kit may further comprise gRNAs specific to a CD70 gene for inserting the CAR-coding sequence into the CD70 gene. In yet other examples, the kit may further comprise gRNAs specific to a Reg1 gene for inserting the CAR-coding sequence into the Reg1 gene. In still other examples, the kit may further comprise gRNAs specific to a TGFBRII gene for inserting the CAR-coding sequence into the TGFBRII gene.

In yet other embodiments, the kit disclosed herein may comprise a population of therapeutic T cells as disclosed for the intended therapeutic purposes.

Any of the kit disclosed herein may further comprise instructions for making the therapeutic T cells, or therapeutic applications of the therapeutic T cells. In some examples, the included instructions may comprise a description of using the gene editing components to genetically engineer one or more of the target genes (e.g., cbl-b and optionally one or more of the additional target genes). In other examples, the included instructions may comprise a description of how to introduce a nucleic acid encoding a CAR construction into the T cells for making therapeutic T cells.

Alternatively, the kit may further comprise instructions for administration of the therapeutic T cells as disclosed herein to achieve the intended activity, e.g., eliminating disease cells targeted by the CAR expressed on the therapeutic T cells. The kit may further comprise a description of selecting a subject suitable for treatment based on identifying whether the subject is in need of the treatment. The instructions relating to the use of the therapeutic T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the therapeutic T cells are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an infusion device for administration of the therapeutic T cells. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B.D. Hames & S.J. Higgins eds.(1985»; Transcription and Translation (B.D. Hames & S.J. Higgins, eds. (1984»; Animal Cell Culture (R.I. Freshney, ed. (1986»; Immobilized Cells and Enzymes (1RL Press, (1986»; and B. Perbal, A practical Guide To Molecular Cloning (1984); F.M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Efficient Disruption of CBLB by Cas9:sgRNA RNPs in T Cells

This example describes efficient editing of the Cbl-B gene in primary human T cells ex vivo using CRISPR/Cas9 gene editing. Genomic segments of the Cbl-B gene containing the CBLB protein coding exons were used as input in gRNA design software. The genomic segments also included flanking splice site acceptor/donor sequences. Desired gRNAs were those that would lead to insertions or deletions in the coding sequence, disrupting the amino acid sequence of Cbl-B leading to out of frame/loss of function allele(s) (referred to as “Cbl-B knockout alleles” or “Cbl-B disrupted alleles”). A number of in silico-identified gRNA spacer sequences targeting the Cbl-B gene were synthesized, and the gRNAs were specifically modified, as indicated in Table 2. While the modified gRNAs in Table 2 were modified with 2′-O-methyl phosphorothioate modifications, unmodified gRNAs, or gRNAs with other modifications, can be used.

Primary human T cells were transfected (electroporated) with a ribonucleoprotein particle (RNP) containing Cas9 nuclease and a synthetic modified sgRNA targeting the Cbl-B gene (sequences in Table 2. Primary human cells not transfected with Cas9 and gRNA were used as controls. Four to six (4-6) days post transfection, cells were: (1) subjected to a TIDE analysis to assess indel frequency, and (2) processed by western blot (primary antibody: antihuman Cbl-B antibody, clone# 16H2L4) to assess Cbl-B expression levels at the cell surface.

A number of gRNAs yielded measurable data by TIDE analysis, as indicated in Table 14 below. Several gRNA sequences yielded indel percentages (editing frequencies) above 80% indicating highly efficient gene editing. The level of Cbl-B protein expression was assessed by western blot to confirm the TIDE analysis data and β-actin was used as a loading control. Table 14.

TABLE 14

TIDE Analysis

| Guide_Name (target site) | Donors | | |
|---|---|---|---|
| | #LPT20-IO-08 | #LPT20-IO-15 | #LPT20-IO-16 |
| CBLB-4 (exon 2) | 5.20% | 7.10% | 4.90% |
| CBLB-5 (exon 2) | 76.40% | 78.30% | 71.10% |
| CBLB-6 (exon 2) | 89.60% | 86.60% | 82.20% |
| CBLB-7 (exon 2) | 78.60% | 79.30% | 72.80% |
| CBLB-T3 (exon 2) | 95% | 91.60% | 91.80% |
| CBLB-T1 (exon 2) | 82.40% | 83% | 79% |
| CBLB-8 (exon 7) | 89.30% | 89.60% | 82.20% |
| CBLB-9 (exon 7) | 3.30% | 5.90% | 3.30% |
| CBLB-10 (exon 7) | 34.40% | 36.30% | 20.70% |
| CBLB-11 (exon 7) | 59.80% | 59.90% | 40.50% |
| CBLB-12 (exon 7) | 94.70% | 92.80% | 90% |
| CBLB-13 (exon 9) | 91.50% | 89.50% | 88.60% |
| CBLB-14 (exon 11) | 78.80% | 82.10% | 74.60% |

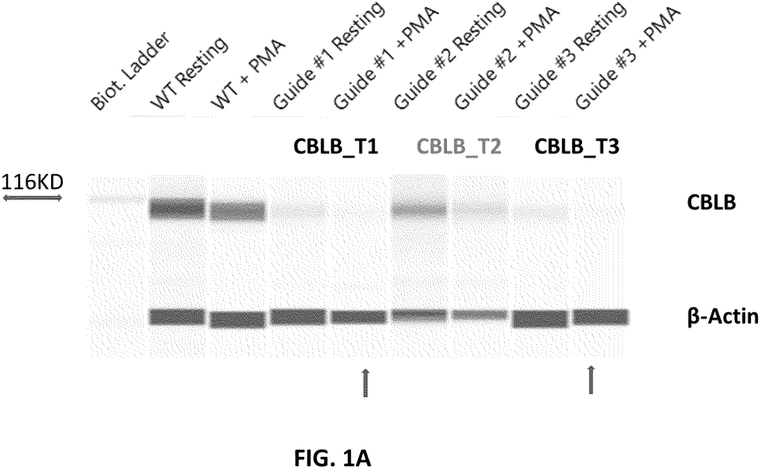

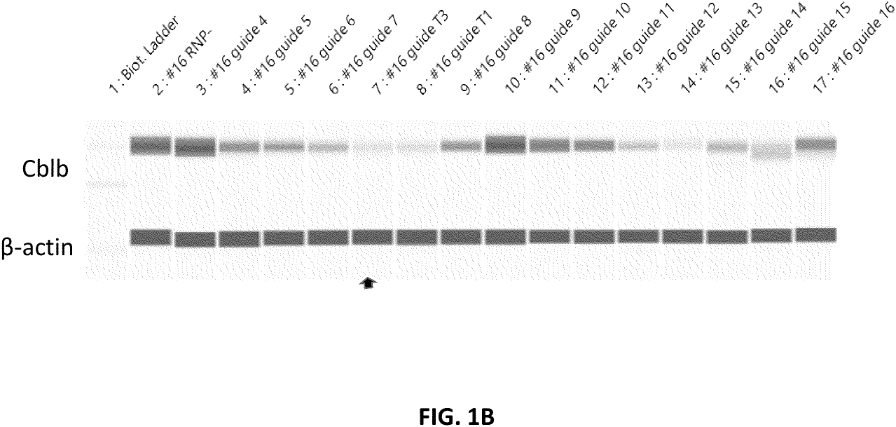

The levels of CBLB protein in cells treated with various gRNAs targeting the cbl-b gene were examined by Westernblot. The results are shown in FIGS. 1A and 1B. A few gRNAs, including CBLB-T1 and CBLB-T3, showed nearly complete knock out of Cbl-B on the T cells, (see the arrows in FIGS. 1A and 1B).

### Example 2: On-Target and Off-Target Editing of CBLB Guide RNAs.

On-target and off-target editing efficiencies of various CBLB-targeting gRNAs were examined following the method disclosed in Example 1 above. Briefly, activated T cells derived from primary human PBMC cells were transfected (electroporated) with a ribonucleoprotein particle (RNP) containing Cas9 nuclease and a synthetic modified sgRNA targeting the CBLB gene or controls (no Cas9, no sgRNA).

For genomic on- and off-target assessment, these electroporation methods were used to generate three cell populations of edited cells from three different donor T cells (termed Donor 1, Donor 2, and Donor 3). Cells were gene edited with each of the guides listed in Table 2, and then collected seven (7) days post transfection. These samples were first analyzed for knockdown of the CBLB protein. The seven sgRNAs with the strongest protein knockdown according to Western, which all achieved 68% or higher knockdown, plus two additional guides with lower Western-detected knockdown, were analyzed with hybrid capture, a method of enrichment of DNA from pre-specified genomic sites, followed by next-generation sequencing. Briefly, on- and off-target sites with homology to each gRNA target site were identified computationally, single-stranded RNA probes were used to enrich these sites from bulk genomic DNA, these enriched sites were sequenced with next-generation sequencing, and the data were analyzed for insertions and deletions indicating repair following CRISPR editing.

TABLE 15

On and Off Target Results by Hybrid Capture

| Guide name | Number of predicted off-target sites tested | On-target mean indel rate by hybrid capture sequencing (%) | Number of detected off-targets [indel rate(s) (%)] | Protein knockdown by Western blot (%) |
|---|---|---|---|---|
| CBLB-T1 | 41 | 99.4 | 3 [4.7, 0.2, 1.6] | 84 |
| CBLB-T3 | 110 | 98.0 | None | 84 |
| CBLB-4 | 261 | - | - | 12 |
| CBLB-5 | 54 | - | - | 66 |
| CBLB-6 | 311 | 94.8 | None | 69 |
| CBLB-7 | 102 | 98.9 | None | 71 |
| CBLB-8 | 56 | 88.8 | None | 68 |
| CBLB-9 | 17 | - | - | 9 |
| CBLB-10 | 21 | - | - | 47 |
| CBLB-11 | 77 | - | - | 55 |
| CBLB-12 | 80 | 95.5 | None | 74 |
| CBLB-13 | 128 | 99.0 | 2 [5.6, 2.9] | 87 |
| CBLB-14 | 152 | - | - | 66 |
| CBLB-15 | 127 | 96.4 | None | 61 |
| CBLB-16 | 89 | 75.8 | None | 52 |

^{a} Average difference between edited and control samples across T cell donors 1, 2, and 3.
Dash (“-”) hybrid capture sequencing was not performed.

### Example 3: Analysis of On-Target Indel Profiles in T Cells.

The data used to quantify off-target editing were also used to quantify and summarize the most frequent on-target indels for all CBLB guides listed in Table 2. This data was generated from hybrid capture of the CBLB locus combined with next-generation sequencing in three donors (termed Donor 1, Donor 2, and Donor 3).

Following gene editing, hybrid capture analysis of the CBLB locus in a population of T cells following CRISPR/Cas9 gene editing to produce CBLB-edited T cells results in specific indel frequencies and edited gene sequences at the CBLB locus (Tables 3-11 deletions as dashes and insertions in bold).

For the purposes of individual sequence quantification from hybrid capture data, sequence reads aligning across the CBLB on-target site and 20 bp upstream and downstream of the predicted cleavage site were selected and considered for indel sequence quantification. From the selected reads, the sequence within 10 bp upstream and downstream of each predicted cleavage site (~3bp upstream of the PAM (Jinek, et al., Science 2012) was quantified as a representative region of on-target non-homologous end joining (NHEJ) editing. The alignments of these on-target gene edited sequences with the associated unedited reference sequences of the targets show detected indels and are presented below in Tables 3 - 11. The reference sequence is centered on the cleavage site with 10 bp in either direction, ending 4 bp 3′ of the PAM. The frequencies of these sequences represent the percent of all sequences spanning the on-target site and including 20 bp upstream and downstream of each cleavage site.

### Example 4: Generation of TRAC-/β2M-/CD70-/Chlb-/anti-CD70 CAR+ Cells and Edit Verification.

Activated primary human T cells were electroporated with Cas9:gRNA RNP complexes and adeno-associated adenoviral vectors (AAVs) to generate both TRAC-/β2M-/CD70-/CBLB- anti-CD70 CAR+ T cells and TRAC-/β2M-/CD70- anti-CD70 CAR+ T cells. Recombinant AAV serotype 6 (AAV6) comprising one of the nucleotide sequences encoding an anti-CD70 CAR (SEQ ID NO: 267), were delivered with Cas9:sgRNA RNPs (1 µM Cas9, 5 µM gRNA) to activated allogeneic human T cells. The following sgRNAs were used: TRAC (SEQ ID NO: 2), β2M (SEQ ID NO: 6), CD70 (SEQ ID: 10) and CBLB (SEQ ID NO. 40).

FACS was used to verify TRAC, B2M, CD70 editing and CAR insertion. About one (1) week post electroporation, cells were processed for flow cytometry to assess TRAC, β2M, CD70 and anti-CD70 CAR expression levels at the cell surface of the edited cell population. For all anti-CD70 CAR T cells and TRAC-/β2M- control cells, >90% of viable cells lacked expression of TCR as well as CD70 and >60% lacked expression of β2M. The anti-CD70 CAR T cells had a high ratio of viable cells expressing the anti-CD70 CAR (>80%).

Western blot analysis was used to verify CBLB editing. About one (1) week post electroporation, one million TRAC-/β2M-/CD70-/CBLB- anti-CD70 CAR+ T-cells and one million unedited T-cells from the same donor were removed from culture and transferred to 1.5mL microcentrifuge tubes. Cells were spun down in a tabletop microcentrifuge at 300g for 10 minutes. The resulting supernatant was removed, and the pelleted cells were washed with 1,000uL dPBS. Cells were then spun once more under the same conditions described previously. The resulting supernatant was removed, and the pelleted cells were transferred to a -80C freezer and left overnight.

The frozen cell pellets were then resuspended with 100uL RIPA Lysis and Extraction Buffer (Thermo Fisher Scientific, catalog # 89900) supplemented with HALT Protease Inhibitor Cocktail (Thermo Fisher Scientific 78430, handled per manufacturer’s instructions). The cell suspensions were briefly vortexed and then incubated on ice for 30 minutes. Every five minutes, the cell suspension was briefly vortexed and then placed back on ice. After 30 minutes, the cell suspensions were transferred to a tabletop microcentrifuge tube chilled to 4C and spun at 13,000g for 10 minutes. The resulting supernatant protein lysate was carefully pipetted and transferred to a separate 1.5mL microcentrifuge tube. The protein lysate was quantified using Thermo Fisher Scientific Nanodrop One (catalog # ND-ONEC-W) via A280 absorbance. 3.2ug of 0.8ug/uL protein lysate was loaded onto a ProteinSimple WES with a 12-230 kDa Separation Module (ProteinSimple, catalog # SM-W001) per manufacturer’s instructions. The WES was loaded with the anti-CBLB rabbit monoclonal antibody clone D3C12 (Cell Signaling Technology, catalog # 9498) and the anti-(3-Actin rabbit monoclonal antibody clone D6A8 (Cell Signaling Technology, catalog # 8457). Both antibodies were diluted 1:100 with the WES antibody diluent. As a protein load control, both TRAC-/β2M-/CBLB- anti-CD70 CAR+ T-cells and unedited T-cells showed similar amounts of β-Actin protein, as shown by the similar ~45kDa bands in both samples. The cells edited with the CBLB gRNA showed significantly less CBLB protein as demonstrated by the significantly lack of a ~109kDa band.

### Example 5: Cytotoxicity Effects of TRAC-/β2M-/CD70-/Chlb-/anti-CD70 CAR+ Cells

Allogeneic human T cells that lack expression of the TRAC gene, β2M gene and CD70 gene, and express a chimeric antigen receptor (CAR) targeting CD70 were produced. The edited CAR T cells further comprised knock out of Cblb gene. As in the examples above, activated human T cells were electroporated with Cas9:sgRNA RNPs (1 µM Cas9, 5 µM gRNA), followed by incubation with a recombinant adeno-associated adenoviral vectors, serotype 6 (AAV6) (MOI 50, 000).

A cell killing assay was used to assess the ability of the TRAC-/β2M-/CD70-/Cblb-/anti-CD70 CAR+ cells to kill CD70+ adherent renal cell carcinoma (RCC)-derived cell lines (A498 and Caki-1 cell lines). Adherent cells were seeded in 96-well plates at 50,000 cells per well and incubated overnight at 37°C. The next day edited anti-CD70 CAR T cells (cultured until day 12 post HDR) were added to the wells containing target cells at 0.1:1, 0.25:1, 0.5:1 or 1:1 CAR T:Target cell ratios. After 24 hours co-culture, CAR T cells were removed from the culture by aspiration and 100 µL Cell titer-Glo (Promega) was added to each well of the plate to assess the number of remaining viable target cells. The amount of light emitted per well was then quantified using a plate reader.

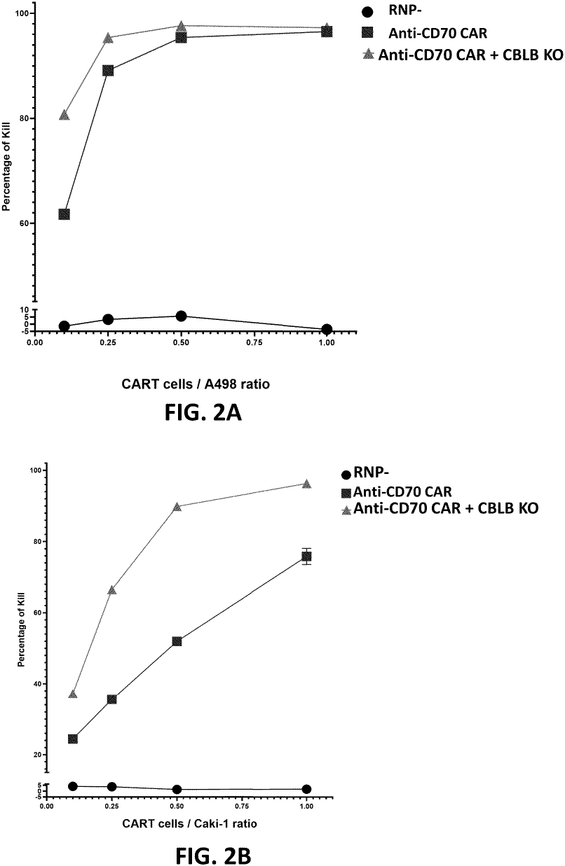

Cells with Cblb disruption exhibited a more potent cell killing of RCC-derived cells (A498 and Caki-1) following 24-hour co-incubation. The anti-CD70 CAR T cells at day 12 post HDR demonstrated significantly higher potency when Cblb was knocked out (FIGS. 2A and 2B).

### Example 6: Effector Cytokine Secretion in TRAC-/β2M-/CD70-/Chlb-/anti-CD70 CAR+ Cells

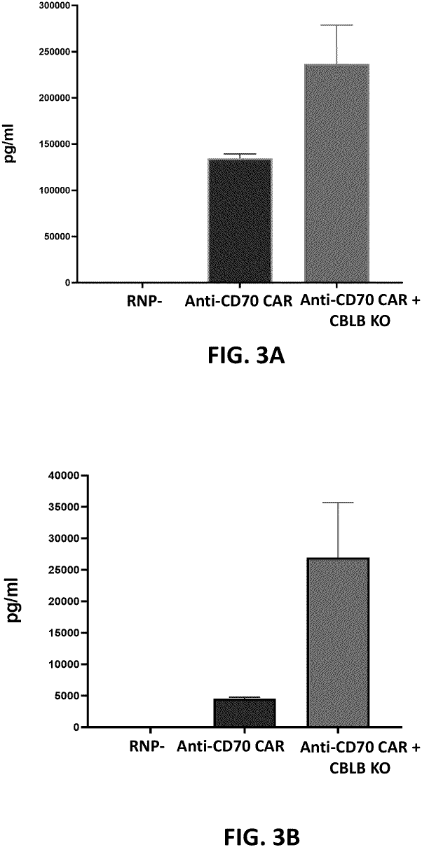

To verify effector cytokine secretion in the presence of target positive cell, supernatants from the cytotoxicity assay were assessed for cytokines using the Luminex platform. MILLIPLEX Human Cytokine/Chemokine Magnetic Bead Panel containing IFN-γ and IL-2 was used to quantify concentrations of each analyte in samples from the cytotoxicity assay. The assay was conducted following manufacturer’s protocol. The samples were read using the LUMINEX 100/200 instrument with XPONENT software and data acquisition and analysis was completed using Sigma-Aldrich Belysa software. The Median Fluorescent Intensity (MFI) data was automatically analyzed using a 5-parameter logistic curve-fitting method for calculating the cytokine concentration measured in the unknown samples. As shown in FIGS. 3A and 3B, TRAC-/02M-/CD70-/CBLB- anti-CD70 CAR+ cells and TRAC-/β2M-/CD70-anti-CD70 CAR+ cells secreted IFN-γ and IL-2 when co-cultured with the target cell line Caki-1 (CAR-T/Caki-1 =0.5) at levels significantly above background (2KO/AAV neg T cells co-cultured with the target cell lines). These data demonstrate that CAR T-cells with the CBLB disruption are capable of secreting effector cytokines in the presence of antigen positive cells.

### Example 7: Disruption of Cblb Increases CAR T Cell Persistence In Vivo.

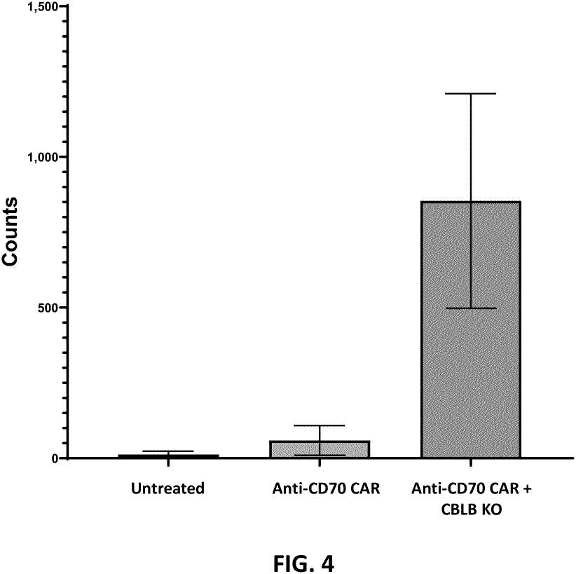

Blood samples were taken from mice with Caki-1 RCC tumors, 44 days after CAR T administration. Briefly, 100ul of mouse whole blood was collected via submandibular vein. Red blood cell lysis buffer was used to achieve optimal lysis of erythrocytes with minimal effect on lymphocytes. Human CD45 and mouse CD45 were used as a biomarker to separate human and mouse cells by FACS. The blood samples were evaluated by flow cytometry looking for absolute CAR T counts. An anti-CD70 CAR anti-idiotype antibody was used to detect CAR T cells. The results demonstrate that the addition of the Cblb gene edit significantly increases TRAC-/β2M-/CD70-/CBLB- anti-CD70 CAR+ cells (Anti-CD70 CAR +CBLB KO) cells persistence in vivo compared to the TRAC-/β2M-/CD70- anti-CD70 CAR+ T cells without Cblb edit (FIG. 4).

### Example 8: Generation of TRAC-/β2M-/CBLB- anti-BCMA CAR+ T Cells and Edit Verification.

Activated primary human T cells were electroporated with Cas9:gRNA RNP complexes and adeno-associated adenoviral vectors (AAVs) to generate both TRAC-/β2M-/ Casitas B-lineage lymphoma proto-oncogene-b negative (CBLB-) anti-B-cell maturation antigen (anti-BCMA) CAR+ T cells and TRAC-/β2M- anti-BCMA CAR+ T cells. Recombinant AAV serotype 6 (AAV6) comprising one of the nucleotide sequences encoding an anti-BCMA CAR (SEQ ID NO: 273), were delivered with Cas9:sgRNA RNPs (1 µM Cas9, 5 µM gRNA) to activated allogeneic human T cells. The following sgRNAs were used: TRAC (SEQ ID NO: 2), β2M (SEQ ID NO: 6), and CBLB (SEQ ID NO. 40) The unmodified versions (or other modified versions) of the gRNAs may also be used (Table 2).

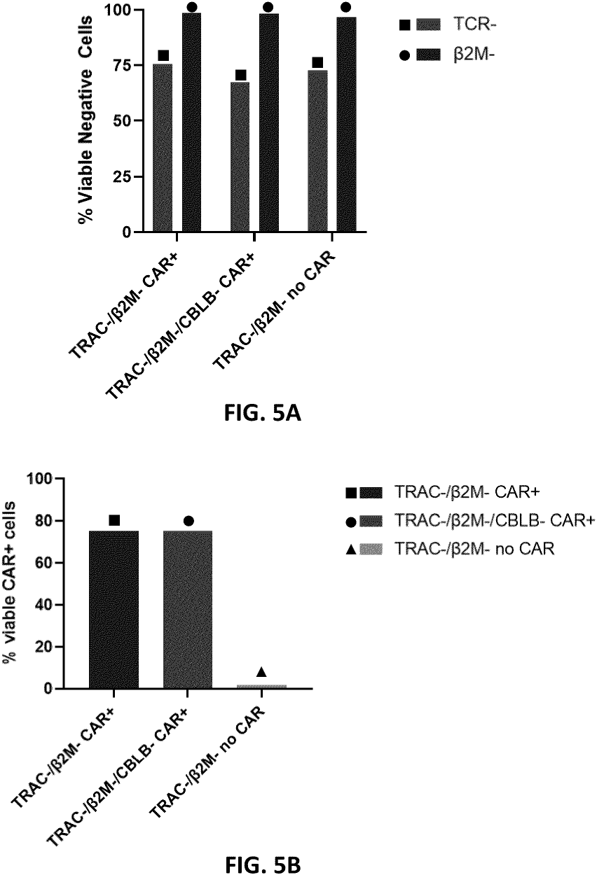

FACS was used to verify TRAC and β2M, CD70 editing and CAR insertion. About one (1) week post electroporation, cells were processed for flow cytometry to assess TRAC, β2M, and anti-BCMA CAR expression levels at the cell surface of the edited cell population. For all anti-BCMA CAR T cells and TRAC-/β2M- control cells, >90% of viable cells lacked expression of TCR and >60% lacked expression of β2M (FIG. 5A). The anti-BCMA CAR T cells had a high ratio of viable cells expressing the anti-BCMA CAR (>70%) (FIG. 5B).

Western blot analysis was used to verify CBLB editing. About one (1) week post electroporation, one million TRAC-/β2M-/CBLB- anti-BCMA CAR+ T-cells and one million unedited T-cells from the same donor were removed from culture and transferred to 1.5mL microcentrifuge tubes. Cells were centrifuged in a tabletop microcentrifuge at 300×g for 10 minutes. The resulting supernatant was removed, and the pelleted cells were washed with 1,000uL dPBS. Cells were then centrifuged once more as described above. The resulting supernatant was removed, and the pelleted cells transferred to a -80°C freezer and left overnight.

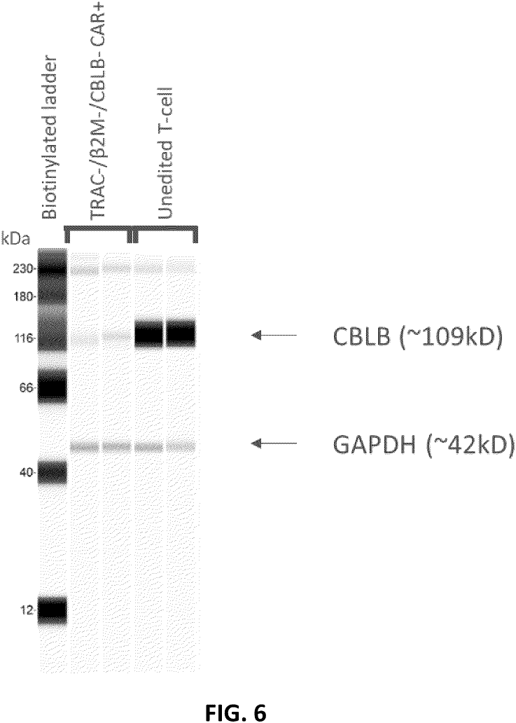

The frozen cell pellets were then resuspended with 100µL RIPA Lysis and Extraction Buffer (Thermo Fisher Scientific, catalog # 89900) supplemented with HALT Protease Inhibitor Cocktail (Thermo Fisher Scientific, catalog # 78430, handled per manufacturer’s instructions). The cell suspensions were briefly vortexed and then incubated on ice for 30 minutes. Every five minutes, the cell suspension was briefly vortexed and then placed back on ice. After 30 minutes, the cell suspensions were transferred to a tabletop microcentrifuge tube chilled to 4C and spun at 13,000g for 10 minutes. The resulting supernatant protein lysate was carefully pipetted and transferred to a separate 1.5mL microcentrifuge tube. The protein lysate was quantified using Thermo Fisher Scientific Nanodrop One^{C} (catalog # ND-ONEC-W) via A280 absorbance. 3.2ug of 0.8ug/uL protein lysate was loaded onto a ProteinSimple WES with a 12-230 kDa Separation Module (ProteinSimple, catalog # SM-W001) per manufacturer’s instructions. The WES was loaded with the anti-CBLB rabbit monoclonal antibody clone D3C12 (Cell Signaling Technology, catalog # 9498) and the anti-GAPDH rabbit monoclonal antibody clone 14C10 (Cell Signaling Technology, catalog # 2118). Both antibodies were diluted 1:100 with the WES antibody diluent. As a protein load control, both TRAC-/β2M-/CBLB- anti-BCMA CAR+ T-cells and unedited T-cells showed similar amounts of GAPDH protein, as shown by the similar ~42kDa bands in both samples (FIG. 6). The cells edited with the CBLB gRNA showed significantly less CBLB protein as demonstrated by the significantly lack of a ~109kDa band.

### Example 9: Cytotoxicity Effects of TRAC-/β2M-/CBLB- Anti-BCMA CAR+ T Cells.

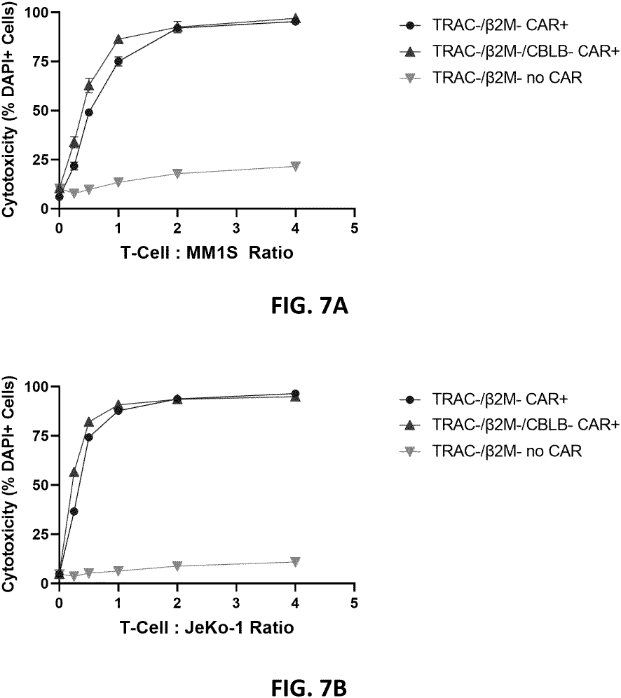

A cell killing (cytotoxicity) assay was used to assess the ability of the TRAC-/β2M-/CBLB- anti-BCMA CAR+ T cells to cause cellular lysis in two target cell lines, MM.1S and JeKo-1. Do demonstrate specific cytotoxicity of the CAR+ T-cells, CAR- T-cells were used as a negative control. The aforementioned target cell lines were stained with eBioscience’s Cell Proliferation Dye EFLUOR 670 per manufacturer’s instructions and seeded into 96-well plates at 50,000 cells per well. Next, T-cells were added to the wells containing target cells at ratios o, 4:1, 2:1, 1:1 or 0.5:1 T cell:target cell. TRAC-/β2M- T cells were used as a negative control. After approximately 4 hours for MM.1S and 24 hours for JeKo-1, cell-containing 96-well plates were centrifuged in a tabletop microcentrifuge at 300×g for 10 minutes and 100µL of supernatant was removed for cytokine quantification (see below). The remaining supernatant was removed and T-cells were resuspended and stained with 150µL of dPBS+0.5% BSA supplemented with 5ug/mL DAPI (Invitrogen, catalog # D3571, handled per manufacturer’s instructions). The cell-containing 96-well plates were then incubated for 15 minutes while protected from light. Target cells were identified via EFLUOR-based fluorescence and then divided into live and dead cells based on their DAPI fluorescence using a fluorescence activated cell sorter (FACS). FIG. 7A and FIG. 7B show the results for cytotoxicity analysis of TRAC-/β2M-/CBLB- anti-BCMA CAR+ cells for exhibited greater cytotoxicity towards the MM.1S and JeKo-1 cells respectively.

### Example 10: Effector Cytokine Secretion in TRAC-/β2M-/CBLB- Anti-BCMA CAR+ T Cells.

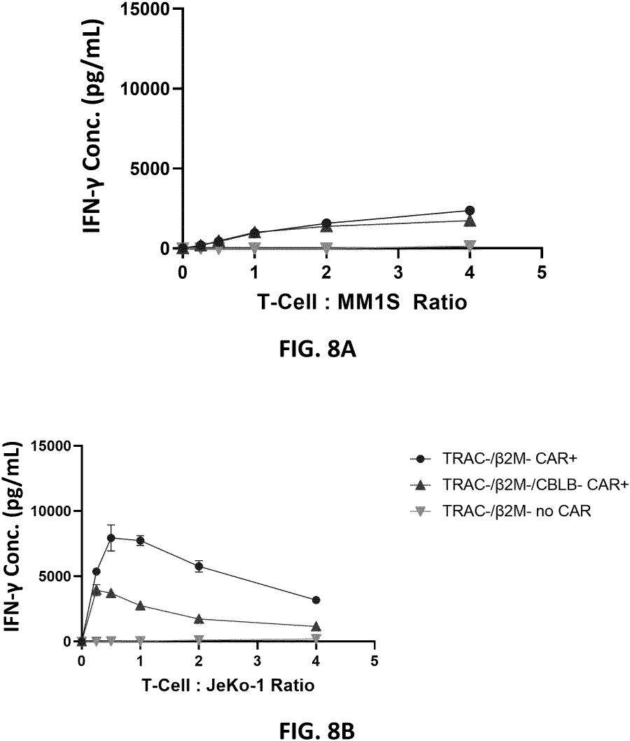

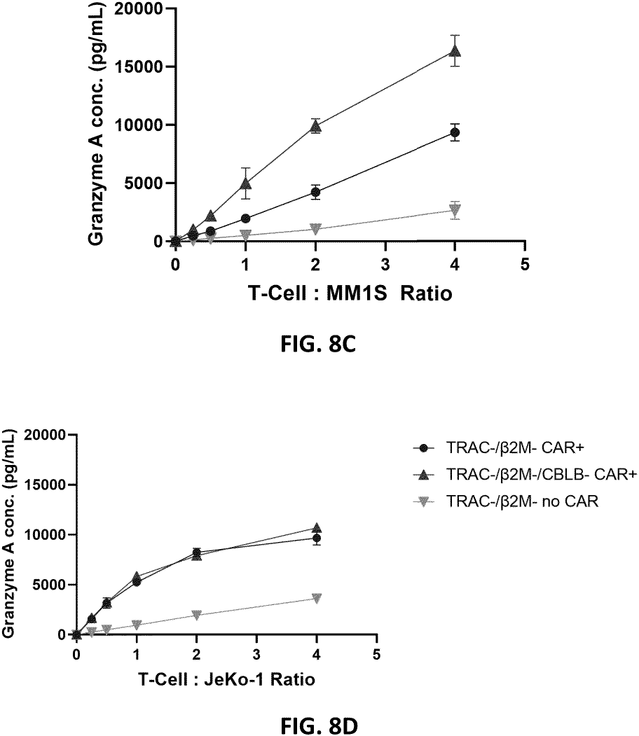

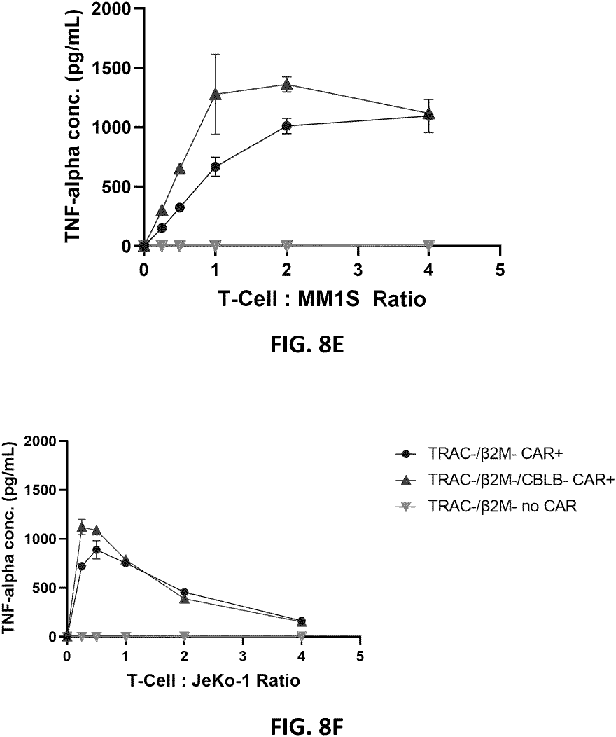

To verify effector cytokine secretion in the presence of target positive cell, supernatants from the cytotoxicity assay were assessed for cytokines using the Luminex platform. The Human XL Cytokine Luminex Performance Base Kit (R&D Systems, catalog # LUXLM000) containing 15 unique species of magnetic microspheres that bind TNF-alpha, IL-6, IL-10, CCL2/MCP-1, Angiopoietin-2, IL-7, CCL3/MIP-1 alpha, CCL4/MIP-1 beta, IL-17/IL-17A, Granzyme A, CXCL9/MIG, IL-5, IFN-γ, IL-15, and BCMA was used to quantify concentrations of each aforementioned analyte in samples from the cytotoxicity assay. The assay was conducted following manufacturer’s protocol. The samples were read using the LUMINEX 100/200 instrument with XPONENT software and data acquisition and analysis was completed using Sigma-Aldrich BELYSA software. The Median Fluorescent Intensity (MFI) data was automatically analyzed using a 5-parameter logistic curve-fitting method for calculating the cytokine concentration measured in the unknown samples. As shown in FIGS. 4A-3F, TRAC-/β2M-/CBLB- anti-BCMA CAR+ cells and TRAC-/β2M- anti-BCMA CAR+ cells secreted IFN-γ (FIGS. 8A-8B), Granzyme A (FIGS. 8C-8D), and TNF-α (FIGS. 8E-8F) when co-cultured with the target cells lines MM.1S and JeKo-1, at levels significantly above background TRAC-/β2M- no CAR cells co-cultured with the target cell lines). These data demonstrate that CAR T-cells with disrupted CBLB are capable of secreting effector cytokines in the presence of antigen positive cells.

### Example 11: In Vivo Effect of CBLB KO on Allogeneic CAR T Cells in the Subcutaneous RPMI-8226 Multiple Myeloma Tumor Xenograft Model.

A subcutaneous mouse tumor model was utilized to assess the in vivo efficacy of allogeneic TRAC-/B2M-/anti-BCMA CAR+ T cells (anti-BCMA CAR T cells) with or without editing of the CBLB locus. The CBLB gene was edited via CRISPR/Cas-mediated gene editing using CBLB T3 (SEQ ID NO. 39). The anti-BCMA CAR T cells express an anti-BCMA CAR comprising the amino acid sequence of SEQ ID NO: 265.

A subcutaneous tumor mouse xenograft model was employed in which, multiple myeloma derived RPMI-8226 tumor cells were implanted in autoimmune non-obese-type diabetes (NOG) mice. Efficacy of the anti-BCMA CAR T cells was evaluated in the subcutaneous xenograft model using methods employed by Translations Drug Development, LLC (Scottsdale, AZ) and described herein. In brief, twenty-five (25) 5-8-week-old female CIEA NOG (NOD.Cg-PrkdcscidI12rgtmlSug/JicTac) mice were individually housed in ventilated microisolator cages, maintained under pathogen-free conditions, 5-7 days prior to the start of the study. On day 1, mice received a subcutaneous inoculation of 1x10⁷ RPMI-8226 cells/mouse in the right hind flank. Nine days later (Day 10), the tumor inoculation sites were inspected to determine if the tumors were palpable. After confirming palpability, the mice were further divided into 5 treatment groups as shown in Table 16. The four treatment groups received a single 200 ul intravenous dose of anti-BCMA CAR+ T cells at the doses specified in Table 16.

TABLE 16

Treatment groups for the RMPI-8226 xenograft study

| Group | CAR T cells (i.v.) | N |
|---|---|---|
| 1 | NA | 5 |
| 2 | anti-BCMA CAR/TRAC-/β2M- (3.5e5 CAR+) | 5 |
| 3 | anti-BCMA CAR/TRAC-/β2M- (1e6 CAR+) | 5 |
| 4 | anti-BCMA CAR/TRAC-/β2M-/CBLB- (3.5e5 CAR+) | 5 |
| 5 | anti-BCMA CAR/TRAC-/β2M-/CBLB- (1e6 CAR+) | 5 |

During the course of the study, the mice were observed daily, and tumor volume and body weight measured twice weekly (about every 3-4 days) starting on Day 10. A significant endpoint was the time to peri-morbidity and the effect of T-cell engraftment was also assessed. The percentage of animal mortality and time to death were recorded for every group in the study. Mice were euthanized prior to reaching a moribund state. Mice are defined as moribund and sacrificed if one or more of the following criteria were met:
1. Loss of body weight of 20% or greater sustained for a period of greater than 1 week;
2. Tumors that inhibit normal physiological function such as eating, drinking, mobility and ability to urinate and or defecate;
3. Prolonged, excessive diarrhea leading to excessive weight loss (>20%);
4. Persistent wheezing and respiratory distress; or
5. Prolonged or excessive pain or distress as defined by clinical observations such as, prostration, hunched posture, paralysis/paresis, distended abdomen, ulcerations, abscesses, seizures and/or hemorrhages.

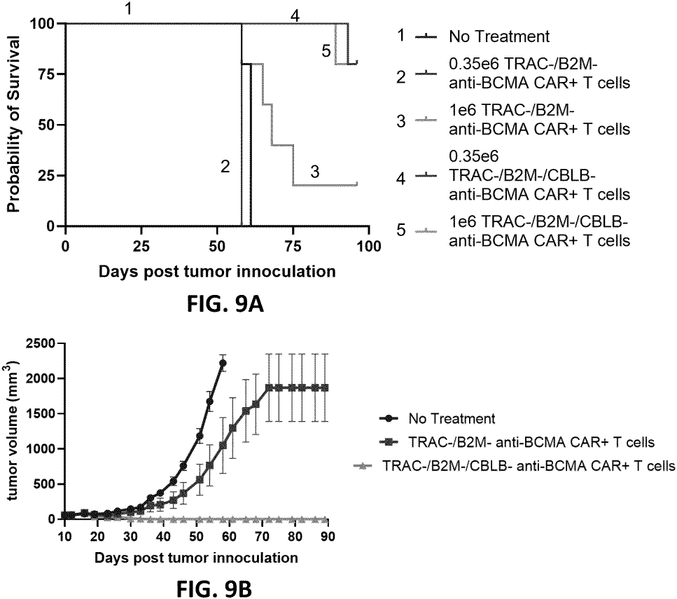

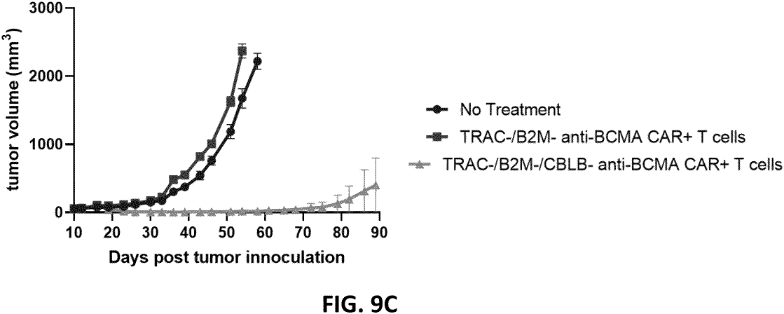

Mice in groups receiving TRAC-/B2M-/CBLB- anti-BCMA CAR+ T cells saw an increase in survival relative to both untreated mice and mice treated with TRAC-/B2M- anti-BCMA CAR+ T-cells (FIG. 9A). This effect could be seen at both tested doses. Mice receiving either 1×10⁶ or 3.5×10⁵ TRAC-/B2M-/CBLB-/anti-BCMA CAR+ T cells showed complete tumor regression (FIGS. 9B-9C), with only one mouse showing tumor regrowth in the 3.5×10⁵ dose group after day 70. No mice receiving TRAC-/B2M- anti-BCMA CAR+ T-cells showed complete tumor regression at either dose level. These data demonstrate that the CBLB disruption in CAR T cells increases efficacy of CAR T cells in a mouse xenograft model.

### Example 12: In Vivo Effect of CBLB, TGFBRII, and Regnase-1 Disruptions on Allogeneic CAR T Cells in a Subcutaneous lung Tumor Xenograft Model with rechallenge.

A subcutaneous mouse tumor model was utilized to assess the in vivo efficacy of allogeneic TRAC-/B2M-/anti-CD70 CAR+ T cells (anti-CD70 CAR T cells) with or without editing of the CBLB locus, and with or without additional potency edits of the TGFBRII and Regnase 1 (Reg1) loci. The CBLB gene was edited via CRISPR/Cas-mediated gene editing using CBLB T3 (SEQ ID NO. 40).

A subcutaneous tumor mouse xenograft model was employed in which, lung NCI-H1975 lung tumor cells were implanted in the right flank of autoimmune non-obese-type diabetes (NOD) mice. On day 1, mice received a subcutaneous inoculation of 5×10⁶ NCI-H1975 cells/mouse (0.1 mL 50% media/50% Matrigel^{®} mixture containing the tumor cells) in the right hind flank. Nine days later (Day 10), the tumor inoculation sites were inspected to determine if the tumors were palpable. After confirming palpability, the mice with tumor size of 64-112 mm³ were randomly assigned to 5 treatment groups as shown in Table 17. The mean tumor size of each group is 92-93 mm³. The four treatment groups received a single 200 ul intravenous dose of anti-CD70 CAR+ T cells at the doses specified in Table 17. At day 32, the mice were rechallenged with a different tumor, ACHN injected in the left flank at 1×10⁷ CAR-T cells per mouse (in 0.1 mL of a 50% media/50% Matrigel® mixture containing a suspension of the CAR-T cells).

TABLE 17

Treatment groups for the tumor xenograft study

| Group | CAR T cells (i.v.) | Designation |
|---|---|---|
| 1 | NA | Control |
| 2 | anti-CD70 CAR/TRAC-/β2M- | Anti-CD70 CAR control |
| 3 | anti-CD70 CAR/TRAC-/β2M-/CBLB- | Anti-CD70+CBLB KO |
| 4 | anti-CD70 CAR/TRAC-/β2M-/CBLB-/TBGFR2- | Anti-CD70+CBLB+TGFBR2 KO |
| 5 | anti-CD70 CAR/TRAC-/β2M-/CBLB-/TGFBR2-/Regnase-1- | Anti-CD70+CBLB+TGFBR2+Reg-1 KO |

During the course of the study, the mice were observed daily, and tumor measured twice weekly (about every 3-4 days) starting on Day 10 (Day 1 being the CAR-T cell infusion day and the tumor inoculation day is -10 day).

Mice in all groups receiving anti-CD70 CAR+ T cells showed complete tumor regression of the primary tumor, NCI-H1975 (Table 18) compared to the control. ‘N/A’ indicates that the mice had to be euthanized.

TABLE 18

Tumor volumes for primary NCI-H1975 tumor (mm³)

| Days | Control | Anti-CD70 CAR control | Anti-CD70+CBLB KO | Anti-CD70+CBLB+ TGFBR2 KO | Anti-CD70+CBLB+TGFBR2+ Reg-1 KO |
|---|---|---|---|---|---|
| 1 | 93 | 92 | 93 | 93 | 93 |
| 3 | 136 | 117 | 119 | 125 | 123 |
| 7 | 303 | 104 | 158 | 147 | 153 |
| 10 | 485 | 43 | 113 | 96 | 76 |
| 14 | 874 | 15 | 39 | 35 | 24 |
| 17 | 1409 | 11 | 19 | 25 | 9 |
| 21 | 1593 | 11 | 17 | 22 | 7 |
| 24 | 2421 | 1 | 9 | 12 | 2 |
| 28 | N/A | 0 | 2 | 6 | 1 |
| 31 | N/A | 0 | 1 | 5 | 1 |
| 35 | N/A | 0 | 0 | 2 | 0 |
| 38 | N/A | 0 | 0 | 3 | 0 |
| 42 | N/A | 0 | 0 | 2 | 0 |
| 45 | N/A | 0 | 0 | 1 | 0 |
| 49 | N/A | 0 | 0 | 1 | 0 |

At day 32, the secondary tumor, ACHN was administered and tumor volumes for this rechallenge were monitored. Days are listed as from the time of the primary tumor challenge (tumor inoculation: -10 day; CAR-T cell infusion: Day 1). Mice in all groups receiving anti-CD70 CAR+ T cells showed tumor regression of the ACHN tumors (Table 19). It was observed that CBLB edit improved the efficacy of CAR T cells alone, as well as in combination with TGFBR2 edit and TGFBR2+Regnase-1 edits.

TABLE 19

Tumor volumes for secondary ACHN tumor (mm³)

| Days | Control | Anti-CD70 CAR control | Anti-CD70+CBLB KO | Anti-CD70+CBLB+ TGFBR2 KO | Anti-CD70+CBLB+TGFBR2+ Reg-1 KO |
|---|---|---|---|---|---|
| 31 | 0 | 0 | 0 | 0 | 0 |
| 35 | 67 | 68 | 64 | 69 | 59 |
| 38 | 73 | 79 | 73 | 82 | 46 |
| 42 | 89 | 42 | 42 | 37 | 10 |
| 45 | 124 | 52 | 41 | 17 | 4 |
| 49 | 152 | 63 | 45 | 2 | 1 |

These data demonstrate that the CBLB disruption in CAR T cells increases efficacy of CAR T cells in a mouse xenograft model.

### Sequence Tables

The following tables provide details for the various nucleotide and amino acid sequences disclosed herein.

TABLE 1

sgRNA Sequences and Target Sequences
gRNA Sequences

| Name | Unmodified Sequence | Modified Sequence |
|---|---|---|
| TRAC sgRNA | AGAGCAACAGUGCUGUGG CCguuuuagagcuagaaauagcaagu uaaaauaaggcuaguccguuaucaacu ugaaaaaguggcaccgagucggugcU UUU (SEQ ID NO: 1) | A*G*A*GCAACAGUGCUGU GGCCguuuuagagcuagaaauagca aguuaaaauaaggcuaguccguuauca acuugaaaaaguggcaccgagucggug cU*U*U*U (SEQ ID NO: 2) |
| TRAC sgRNA spacer | AGAGCAACAGUGCUGUGG CC (SEQ ID NO: 3) | A*G*A*GCAACAGUGCUGU GGCC (SEQ ID NO: 4) |
| β2M sgRNA | GCUACUCUCUCUUUCUGG CCguuuuagagcuagaaauagcaagu uaaaauaaggcuaguccguuaucaacu ugaaaaaguggcaccgagucggugcU UUU (SEQ ID NO: 5) | G*C*U*ACUCUCUCUUUCU GGCCguuuuagagcuagaaauagca aguuaaaauaaggcuaguccguuauca acuugaaaaaguggcaccgagucggug cU*U*U*U (SEQ ID NO: 6) |
| β2M sgRNA spacer | GCUACUCUCUCUUUCUGG CC (SEQ ID NO: 7) | G*C*U*ACUCUCUCUUUCU GGCC (SEQ ID NO: 8) |
| CD70 sgRNA | GCUUUGGUCCCAUUGGUC GCguuuuagagcuagaaauagcaagu uaaaauaaggcuaguccguuaucaacu ugaaaaaguggcaccgagucggugcU UUU (SEQ ID NO: 9) | G*C*U*UUGGUCCCAUUGG UCGCguuuuagagcuagaaauagca aguuaaaauaaggcuaguccguuauca acuugaaaaaguggcaccgagucggug cU*U*U*U (SEQ ID NO: 10) |
| CD70 sgRNA spacer | GCUUUGGUCCCAUUGGUC GC (SEQ ID NO: 11) | G*C*U*UUGGUCCCAUUGG UCGC (SEQ ID NO: 12) |
| Target Sequences | | |
| Guide Name | Target Sequence (PAM) | Target Sequence - Without PAM |
| TRAC sgRNA | AGAGCAACAGTGCTGTGG CC (TGG) (SEQ ID NO: 13) | AGAGCAACAGTGCTGTGG CC (SEQ ID NO: 14) |
| β2M sgRNA | GCTACTCTCTCTTTCTGGC C (TGG) (SEQ ID NO: 15) | GCTACTCTCTCTTTCTGGC C (SEQ ID NO: 16) |
| CD70 sgRNA | GCTTTGGTCCCATTGGTCG C (GGG) (SEQ ID NO: 17) | GCTTTGGTCCCATTGGTCG C (SEQ ID NO: 18) |

Exemplary sgRNA Formula
nnnnnnnnnnnnnnnnnnnnguuuuagagcuagaaauagcaaguuaaaauaaggcuaguccguuaucaacuugaaaaaguggca ccgagucggugcuuuu (SEQ ID NO: 19)
nnnnnnnnnnnnnnnnnnnnguuuuagagcuagaaauagcaaguuaaaauaaggcuaguccguuaucaacuugaaaaaguggca ccgagucggugc (SEQ ID NO: 20)
n₍₁₇₋₃₀₎guuuuagagcuagaaauagcaaguuaaaauaaggcuaguccguuaucaacuugaaaaaguggcaccgagucggugcu₍₁₋₈₎ (SEQ ID NO: 21)
Exemplary Cas9 sequence (spCas9)
MDKKYSIGLDIGTNSVGWAVITDEYKVPSKKFKVLGNTDRHSIKKNLIGALLFDSGETAEA TRLKRTARRRYTRRKNRICYLQEIFSNEMAKVDDSFFHRLEESFLVEEDKKHERHPIFGNIV DEVAYHEKYPTIYHLRKKLVDSTDKADLRLIYLALAHMIKFRGHFLIEGDLNPDNSDVDKL FIQLVQTYNQLFEENPINASGVDAKAILSARLSKSRRLENLIAQLPGEKKNGLFGNLIALSLG LTPNFKSNFDLAEDAKLQLSKDTYDDDLDNLLAQIGDQYADLFLAAKNLSDAILLSDILRV NTEITKAPLSASMIKRYDEHHQDLTLLKALVRQQLPEKYKEIFFDQSKNGYAGYIDGGASQ EEFYKFIKPILEKMDGTEELLVKLNREDLLRKQRTFDNGSIPHQIHLGELHAILRRQEDFYPF LKDNREKIEKILTFRIPYYVGPLARGNSRFAWMTRKSEETITPWNFEEVVDKGASAQSFIER MTNFDKNLPNEKVLPKHSLLYEYFTVYNELTKVKYVTEGMRKPAFLSGEQKKAIVDLLFK TNRKVTVKQLKEDYFKKIECFDSVEISGVEDRFNASLGTYHDLLKIIKDKDFLDNEENEDIL EDIVLTLTLFEDREMIEERLKTYAHLFDDKVMKQLKRRRYTGWGRLSRKLINGIRDKQSGK TILDFLKSDGFANRNFMQLIHDDSLTFKEDIQKAQVSGQGDSLHEHIANLAGSPAIKKGILQ TVKVVDELVKVMGRHKPENIVIEMARENQTTQKGQKNSRERMKRIEEGIKELGSQILKEHP VENTQLQNEKLYLYYLQNGRDMYVDQELDINRLSDYDVDHIVPQSFLKDDSIDNKVLTRS DKNRGKSDNVPSEEVVKKMKNYWRQLLNAKLITQRKFDNLTKAERGGLSELDKAGFIKR QLVETRQITKHVAQILDSRMNTKYDENDKLIREVKVITLKSKLVSDFRKDFQFYKVREINN YHHAHDAYLNAVVGTALIKKYPKLESEFVYGDYKVYDVRKMIAKSEQEIGKATAKYFFYS NIMNFFKTEITLANGEIRKRPLIETNGETGEIVWDKGRDFATVRKVLSMPQVNIVKKTEVQT GGFSKESILPKRNSDKLIARKKDWDPKKYGGFDSPTVAYSVLVVAKVEKGKSKKLKSVKE LLGITIMERSSFEKNPIDFLEAKGYKEVKKDLIIKLPKYSLFELENGRKRMLASAGELQKGN ELALPSKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVLDATLI HQSITGLYETRIDLSQLGGD (SEQ ID NO: 22)
*: 2′-O-methyl phosphorothioate residue
“n” refers to the spacer sequence at the 5′ end

TABLE 2

sgRNA Sequences and Target Gene Sequences for cbl-b

| Name | Unmodified Sequence | Modified Sequence | Target Sequences (PAM) |
|---|---|---|---|
| CBLB-4 | UACCCAAAAUUCGACCUUUUguuuuagagcuagaaauagcaaguuaaaauaaggcuaguccguuaucaacuuga | U*A*C*CCAAAAUUCGACCUUUUguuuuagagcuagaaauagcaaguuaaaauaaggcuaguccguuaucaacuugaaaaaguggcaccgagu | TACCCAAAATTCGACCTTTT (CGG) (SEQ ID NO: 83) TACCCAAAATTCGACC |
| Name | Unmodified Sequence | Modified Sequence | Target Sequences (PAM) |
| | aaaaguggcaccgagucggugcUUUU (SEQ ID NO: 23) | cggugcU*U*U*U (SEQ ID NO: 24) | TTTT (SEQ ID NO: 84) |
| CBLB-4 spacer | UACCCAAAAUUCGACCUUUU (SEQ ID NO: 25) | U*A*C*CCAAAAUUCGACCUUUU (SEQ ID NO: 26) | |
| CBLB-5 | CCCCGAAAAGGUCGAAUUUUguuuuagagcuagaaauagcaaguuaaaauaaggcuaguccguuaucaacuugaaaaaguggcaccgagucggugcUUUU (SEQ ID NO: 27) | C*C*C*CGAAAAGGUCGAAUUUUguuuuagagcuagaaauagcaaguuaaaauaaggcuaguccguuaucaacuugaaaaaguggcaccgagucggugcU*U*U*U (SEQ ID NO: 28) | CCCCGAAAAGGTCGAATTTT (GGG) (SEQ ID NO: 85) CCCCGAAAAGGTCGAATTTT (SEQ ID NO: 86) |
| CBLB-5 spacer | CCCCGAAAAGGUCGAAUUUU (SEQ ID NO: 29) | C*C*C*CGAAAAGGUCGAAUUUU (SEQ ID NO: 30) | |
| CBLB-6 | GGGUAUUAUUGAUGCUAUUCguuuuagagcuagaaauagcaaguuaaaauaaggcuaguccguuaucaacuugaaaaaguggcaccgagucggugcUUUU (SEQ ID NO: 31) | G*G*G*UAUUAUUGAUGCUAUUCguuuuagagcuagaaauagcaaguuaaaauaaggcuaguccguuaucaacuugaaaaaguggcaccgagucggugcU*U*U*U (SEQ ID NO: 32) | GGGTATTATTGATGCTATTC (AGG) (SEQ ID NO: 87) GGGTATTATTGATGCTATTC (SEQ ID NO: 88) |
| CBLB-6 spacer | GGGUAUUAUUGAUGCUAUUC (SEQ ID NO: 33) | G*G*G*UAUUAUUGAUGCUAUUC (SEQ ID NO: 34) | |
| CBLB-7 | GAUCUGCGGCAGCUUGCUUAguuuuagagcuagaaauagcaaguuaaaauaaggcuaguccguuaucaacuugaaaaaguggcaccgagucggugcUUUU (SEQ ID NO: 35) | G*A*U*CUGCGGCAGCUUGCUUAguuuuagagcuagaaauagcaaguuaaaauaaggcuaguccguuaucaacuugaaaaaguggcaccgagucggugcU*U*U*U (SEQ ID NO: 36) | GATCTGCGGCAGCTTGCTTA (GGG) (SEQ ID NO: 89) GATCTGCGGCAGCTTGCTTA (SEQ ID NO: 90) |
| CBLB-7 spacer | GAUCUGCGGCAGCUUGCUUA (SEQ ID NO: 37) | G*A*U*CUGCGGCAGCUUGCUUA (SEQ ID NO: 38) | |
| CBLB-T3 | AGCAAGCUGCCGCAGAUCGCguuuuagagcuagaaauagcaaguuaaaauaaggcuaguccguuaucaacuugaaaaaguggcaccgagucggugcUUUU (SEQ ID NO: 39) | A*G*C*AAGCUGCCGCAGAUCGCguuuuagagcuagaaauagcaaguuaaaauaaggcuaguccguuaucaacuugaaaaaguggcaccgagucggugcU*U*U*U (SEQ ID NO: 40) | AGCAAGCTGCCGCAGATCGC (AGG) (SEQ ID NO: 91) AGCAAGCTGCCGCAGATCGC (SEQ ID NO: 92) |
| CBLB-T3 spacer | AGCAAGCUGCCGCAGAUCGC (SEQ ID NO: 41) | A*G*C*AAGCUGCCGCAGAUCGC (SEQ ID NO: 42) | |
| Name | Unmodified Sequence | Modified Sequence | Target Sequences (PAM) |
| CBLB-T1 | UGCCGCAGAUCGCA GGACCGguuuuagagcua gaaauagcaaguuaaaauaag gcuaguccguuaucaacuuga aaaaguggcaccgagucggug cUUUU (SEQ ID NO: 43) | U*G*C*CGCAGAUCGCAG GACCGguuuuagagcuagaaaua gcaaguuaaaauaaggcuaguccguu aucaacuugaaaaaguggcaccgagu cggugcU*U*U*U (SEQ ID NO: 44) | TGCCGCAGATCGCAGGACCG (TGG) (SEQ ID NO: 93) TGCCGCAGATCGCAGGACCG (SEQ ID NO: 94) |
| CBLB-T1 spacer | UGCCGCAGAUCGCA GGACCG (SEQ ID NO: 45) | U*G*C*CGCAGAUCGCAG GACCG (SEQ ID NO: 46) | |
| CBLB-8 Exon 7 | CCAAUCGAGUGCAA CUUAACguuuuagagcua gaaauagcaaguuaaaauaag gcuaguccguuaucaacuuga aaaaguggcaccgagucggug cUUUU (SEQ ID NO: 47) | C*C*A*AUCGAGUGCAAC UUAACguuuuagagcuagaaaua gcaaguuaaaauaaggcuaguccguu aucaacuugaaaaaguggcaccgagu cggugcU*U*U*U (SEQ ID NO: 48) | CCAATCGAGTGCAACT TAAC (CGG) (SEQ ID NO: 95) CCAATCGAGTGCAACT TAAC (SEQ ID NO: 96) |
| CBLB-8 spacer | CCAAUCGAGUGCAA CUUAAC (SEQ ID NO: 49) | C*C*A*AUCGAGUGCAAC UUAAC (SEQ ID NO: 50) | |
| CBLB-9 | CCGGUUAAGUUGCA CUCGAUguuuuagagcua gaaauagcaaguuaaaauaag gcuaguccguuaucaacuuga aaaaguggcaccgagucggug cUUUU (SEQ ID NO: 51) | C*C*G*GUUAAGUUGCAC UCGAUguuuuagagcuagaaaua gcaaguuaaaauaaggcuaguccguu aucaacuugaaaaaguggcaccgagu cggugcU*U*U*U (SEQ ID NO: 52) | CCGGTTAAGTTGCACT CGAT (TGG) (SEQ ID NO: 97) CCGGTTAAGTTGCACT CGAT (SEQ ID NO: 98) |
| CBLB-9 spacer | CCGGUUAAGUUGCA CUCGAU (SEQ ID NO: 53) | C*C*G*GUUAAGUUGCAC UCGAU (SEQ ID NO: 54) | |
| CBLB-10 | CGGUUAAGUUGCAC UCGAUUguuuuagagcua gaaauagcaaguuaaaauaag gcuaguccguuaucaacuuga aaaaguggcaccgagucggug cUUUU (SEQ ID NO: 55) | C*G*G*UUAAGUUGCACU CGAUUguuuuagagcuagaaaua gcaaguuaaaauaaggcuaguccguu aucaacuugaaaaaguggcaccgagu cggugcU*U*U*U (SEQ ID NO: 56) | CGGTTAAGTTGCACTC GATT (GGG) (SEQ ID NO: 99) CGGTTAAGTTGCACTC GATT (SEQ ID NO: 100) |
| CBLB-10 spacer | CGGUUAAGUUGCAC UCGAUU (SEQ ID NO: 57) | C*G*G*UUAAGUUGCACU CGAUU (SEQ ID NO: 58) | |
| CBLB-11 | GUUGCACUCGAUUG GGACAGguuuuagagcua gaaauagcaaguuaaaauaag gcuaguccguuaucaacuuga aaaaguggcaccgagucggug cUUUU (SEQ ID NO: 59) | G*U*U*GCACUCGAUUGG GACAGguuuuagagcuagaaaua gcaaguuaaaauaaggcuaguccguu aucaacuugaaaaaguggcaccgagu cggugcU*U*U*U (SEQ ID NO: 60) | GTTGCACTCGATTGGG ACAG (TGG) (SEQ ID NO: 101) GTTGCACTCGATTGGG ACAG (SEQ ID NO: 102) |
| CBLB-11 spacer | GUUGCACUCGAUUG GGACAG (SEQ ID NO: 61) | G*U*U*GCACUCGAUUGG GACAG (SEQ ID NO: 62) | |
| CBLB-12 | UUGCACUCGAUUGG | U*U*G*CACUCGAUUGGG | TTGCACTCGATTGGGA |
| Name | Unmodified Sequence | Modified Sequence | Target Sequences (PAM) |
| | GACAGUguuuuagagcua gaaauagcaaguuaaaauaag gcuaguccguuaucaacuuga aaaaguggcaccgagucggug cUUUU (SEQ ID NO: 63) | ACAGUguuuuagagcuagaaaua gcaaguuaaaauaaggcuaguccguu aucaacuugaaaaaguggcaccgagu cggugcU*U*U*U (SEQ ID NO: 64) | CAGT (GGG) (SEQ ID NO: 103) TTGCACTCGATTGGGA CAGT (SEQ ID NO: 104) |
| CBLB-12 spacer | UUGCACUCGAUUGG GACAGU (SEQ ID NO: 65) | U*U*G*CCACCCACGCGU CGUG (SEQ ID NO: 66) | |
| CBLB-13 | GCACCUCUUGCCUU ACGGCAguuuuagagcua gaaauagcaaguuaaaauaag gcuaguccguuaucaacuuga aaaaguggcaccgagucggug cUUUU (SEQ ID NO: 67) | G*C*A*CCUCUUGCCUUA CGGCAguuuuagagcuagaaaua gcaaguuaaaauaaggcuaguccguu aucaacuugaaaaaguggcaccgagu cggugcU*U*U*U (SEQ ID NO: 68) | GCACCTCTTGCCTTAC GGCA (TGG) (SEQ ID NO: 105) GCACCTCTTGCCTTAC GGCA (SEQ ID NO: 106) |
| CBLB-13 spacer | GCACCUCUUGCCUU ACGGCA (SEQ ID NO: 69) | G*C*A*CCUCUUGCCUUA CGGCA (SEQ ID NO: 70) | |
| CBLB-14 | UUCUGAAUUAGAUC CAGGCGguuuuagagcua gaaauagcaaguuaaaauaag gcuaguccguuaucaacuuga aaaaguggcaccgagucggug cUUUU (SEQ ID NO: 71) | U*U*C*UGAAUUAGAUCC AGGCGguuuuagagcuagaaaua gcaaguuaaaauaaggcuaguccguu aucaacuugaaaaaguggcaccgagu cggugcU*U*U*U (SEQ ID NO: 72) | TTCTGAATTAGATCCA GGCG (AGG) (SEQ ID NO: 107) TTCTGAATTAGATCCA GGCG (SEQ ID NO: 108) |
| CBLB-14 spacer | UUCUGAAUUAGAUC CAGGCG (SEQ ID NO: 73) | U*U*C*UGAAUUAGAUCC AGGCG (SEQ ID NO: 74) | |
| CBLB-15 | UAGUCCCAAACACA UCCCGAguuuuagagcua gaaauagcaaguuaaaauaag gcuaguccguuaucaacuuga aaaaguggcaccgagucggug cUUUU (SEQ ID NO: 75) | U*A*G*UCCCAAACACAU CCCGAguuuuagagcuagaaaua gcaaguuaaaauaaggcuaguccguu aucaacuugaaaaaguggcaccgagu cggugcU*U*U*U (SEQ ID NO: 76) | TAGTCCCAAACACATC CCGA (GGG) (SEQ ID NO: 109) TAGTCCCAAACACATC CCGA (SEQ ID NO: 110) |
| CBLB-15 spacer | UAGUCCCAAACACA UCCCGA (SEQ ID NO: 77) | U*A*G*UCCCAAACACAU CCCGA (SEQ ID NO: 78) | |
| CBLB-16 | UUAGUCCCAAACAC AUCCCGguuuuagagcua gaaauagcaaguuaaaauaag gcuaguccguuaucaacuuga aaaaguggcaccgagucggug cUUUU (SEQ ID NO: 79) | U*U*A*GUCCCAAACACA UCCCGguuuuagagcuagaaaua gcaaguuaaaauaaggcuaguccguu aucaacuugaaaaaguggcaccgagu cggugcU*U*U*U (SEQ ID NO: 80) | TTAGTCCCAAACACAT CCCG (AGG) (SEQ ID NO: 111) TTAGTCCCAAACACAT CCCG (SEQ ID NO: 112) |
| CBLB-16 spacer | UUAGUCCCAAACAC AUCCCG (SEQ ID NO: 81) | U*U*A*GUCCCAAACACA UCCCG (SEQ ID NO: 82) | |

* indicates a nucleotide with a 2′-O-methyl phosphorothioate modification.

TABLE 3

On-Target Gene Edited Sequences with more than 1% Frequency in At Least One Gene Edited T Cell Donor for the CBLB-T1 gRNA
Reference on-target sequence”: GATCGCAGGACCG (TGG) AGAA (SEQ ID NO: 290)

| SEQ ID NO: | Gene edited sequence^{b} SEQ ID NO: | Donor 1 (%) | Donor 2 (%) | Donor 3 (%) | Mean (%) | Std. dev. (%) |
|---|---|---|---|---|---|---|
| 113 | GATCGCAGGAACCGTGGAGAA | 8.5 | 15.5 | 12.0 | 12.0 | 3.5 |
| 114 | GATCGCAGGA-------GAA | 12.4 | 11.1 | 9.0 | 10.8 | 1.7 |
| 115 | GATCGCAGGAC-GTGGAGAA | 9.8 | 6.5 | 8.1 | 8.1 | 1.7 |
| 116 | GATCG--------TGGAGAA | 6.3 | 6.5 | 6.7 | 6.5 | 0.2 |
| 117 | GATCGCAGGA---------- | 4.0 | 5.0 | 3.5 | 4.2 | 0.8 |
| | ---- -- ---- -- ---- ---- | 3.0 | 2.6 | 4.1 | 3.2 | 0.8 |
| 118 | GATCGCAGG-CCGTGGAGAA | 3.3 | 3.2 | 3.7 | 3.4 | 0.3 |
| 119 | GATCGC-----CGTGGAGAA | 3.3 | 2.1 | 1.9 | 2.4 | 0.7 |
| 120 | GATCGCAG--CCGTGGAGAA | 3.2 | 3.1 | 2.7 | 3.0 | 0.3 |
| | GATCGCAG ------------ | 2.0 | 2.8 | 2.8 | 2.6 | 0.5 |
| | ------------- TGGAGAA | 1.4 | 1.8 | 2.2 | 1.8 | 0.4 |
| 121 | ----------CCGTGGAGAA | 2.1 | 1.8 | 1.4 | 1.7 | 0.4 |
| 122 | GATCGCAGGA--GTGGAGAA | 2.0 | 1.6 | 1.4 | 1.7 | 0.3 |
| | GA------------------ | 1.6 | 1.6 | 2.0 | 1.7 | 0.3 |
| 123 | GATCGCAGGA---------A | 2.0 | 1.2 | 1.4 | 1.5 | 0.4 |
| 124 | GATC-------CGTGGAGAA | 1.2 | 1.2 | 1.5 | 1.3 | 0.2 |
| | -----------CGTGGAGAA | 0.7 | 0.7 | 1.4 | 0.9 | 0.4 |
| 125 | GATCGCAGGAC--------- | 1.1 | 1.3 | 1.2 | 1.2 | 0.1 |
| | GA ----------------- A | 0.4 | 1.1 | 0.6 | 0.7 | 0.4 |
| 126 | GATCGCAG-----TGGAGAA | 1.1 | 0.5 | 0.2 | 0.6 | 0.4 |
| 127 | GA--------CCGTGGAGAA | 0.6 | 0.6 | 1.0 | 0.8 | 0.2 |
| 128 | G---------CCGTGGAGAA | 1.0 | 0.6 | 0.8 | 0.8 | 0.2 |

^{a} On-target sequence centered on cleavage site, with 10 bp in either direction. For comparison, the portion of the gRNA target sequence aligning with the Reference on-target sequence is underlined and the PAM is indicated by parenthesis.
^{b} Deletions indicated by dashes (-); insertions indicated by bold

TABLE 4

On-Target Gene Edited Sequences > 1% Frequency in At Least One Gene Edited T Cell Donor for the ZC3HI2A-2 gRNA.
Reference on-target sequence^{a}: TGCCGCAGATCGC (AGG) ACCG (SEQ ID NO: 291)

| SEQ ID NO: | Gene edited sequence^{b} SEQ ID NO: | Donor 1 (%) | Donor 2 (%) | Donor 3 (%) | Mean (%) | Std. dev. (%) |
|---|---|---|---|---|---|---|
| 129 | TGCCGCAG-------GACCG | 36.6 | 33.1 | 31.1 | 33.6 | 2.8 |
| 130 | TGCCGCAGATTCGCAGGAC CG | 12.7 | 10.3 | 15.8 | 12.9 | 2.7 |
| | TGCCG--------------- | 4.0 | 4.3 | 4.9 | 4.4 | 0.5 |
| 131 | TGCCGCAGA-CGCAGGACCG | 3.6 | 4.9 | 3.4 | 4.0 | 0.8 |
| 132 | ----------CGCAGGACCG | 3.4 | 4.3 | 4.1 | 4.0 | 0.5 |
| 133 | TGCCGCAG--CGCAGGACCG | 2.5 | 3.0 | 2.4 | 2.6 | 0.3 |
| | ---- -- ---- -- ---- ---- | 1.6 | 2.1 | 1.4 | 1.7 | 0.4 |
| | ------------- AGGACCG | 1.6 | 2.0 | 1.2 | 1.6 | 0.4 |
| 134 | TGCCGCAGA--------CCG | 1.9 | 1.4 | 1.8 | 1.7 | 0.3 |
| | -----------GCAGGACCG | 1.5 | 1.3 | 1.8 | 1.5 | 0.3 |
| | ---------------GACCG | 1.8 | 1.7 | 0.8 | 1.4 | 0.5 |
| | -------------------G | 1.5 | 1.0 | 1.1 | 1.2 | 0.3 |
| | --------------GGACCG | 1.3 | 1.4 | 1.2 | 1.3 | 0.1 |
| 135 | TGCCGCA---CGCAGGACCG | 0.7 | 1.4 | 1.4 | 1.2 | 0.4 |
| 136 | TGCCGCAGAT-CAGGACCG | 0.6 | 1.3 | 0.8 | 0.9 | 0.4 |
| 137 | TGCCGC----CGCAGGACCG | 0.8 | 1.1 | 1.3 | 1.1 | 0.2 |
| 138 | TGC----------AGGACCG | 1.3 | 1.0 | 0.8 | 1.0 | 0.2 |
| 139 | TGC--------GCAGGACCG | 0.5 | 0.6 | 1.1 | 0.7 | 0.3 |
| 140 | TGCCGCAGAT-----GACCG | 0.8 | 0.9 | 1.0 | 0.9 | 0.1 |

^{a} On-target sequence centered on cleavage site, with 10 bp in either direction. For comparison, the portion of the gRNA target sequence aligning with the Reference on-target sequence is underlined and the PAM is indicated by parenthesis.
^{b} Deletions indicated by dashes (-); insertions indicated by bold

TABLE 5

On-Target Gene Edited Sequences > 1% Frequency in At Least One Gene Edited T Cell Donor for the CBLB-6 gRNA
Reference on-target sequence^{a}: ATTGATGCTATTC (AGG) ATGC (SEQ ID NO: 292)

| SEQ ID NO: | Gene edited sequence^{b} | Donor 1 (%) | Donor 2 (%) | Donor 3 (%) | Mean (%) | Std. dev. (%) |
|---|---|---|---|---|---|---|
| | ATTGATGC------------ | 25.5 | 20.4 | 18.5 | 21.5 | 3.6 |
| 141 | ATTGATGCTATTTCAGGATG C | 18.3 | 16.7 | 17.7 | 17.6 | 0.8 |
| 142 | ATTGATGCTAATTCAGGATG C | 6.2 | 6.4 | 5.2 | 5.9 | 0.6 |
| | ------------CAGGATGC | 3.0 | 3.2 | 4.0 | 3.4 | 0.5 |
| | -------------------- | 2.4 | 3.6 | 2.5 | 2.8 | 0.7 |
| 143 | ATTGATGCT-TTCAGGATGC | 1.7 | 3.2 | 1.3 | 2.1 | 1.0 |
| 144 | ATTGATGCTAT-CAGGATGC | 1.5 | 2.6 | 2.0 | 2.0 | 0.6 |
| 145 | ATTGATGCT--TCAGGATGC | 1.4 | 2.3 | 2.2 | 2.0 | 0.5 |
| 146 | ATTGATGC-----AGGATGC | 2.1 | 1.5 | 1.8 | 1.8 | 0.3 |
| 147 | ATTGATGCTATATTCAGGAT GC | 0.9 | 1.3 | 2.1 | 1.4 | 0.6 |
| 148 | ATTGATGCTA--CAGGATGC | 1.8 | 1.9 | 1.3 | 1.7 | 0.3 |
| 149 | ATT---------CAGGATGC | 1.7 | 1.6 | 1.9 | 1.7 | 0.1 |
| 150 | ATTGAT-----TCAGGATGC | 1.9 | 1.7 | 0.8 | 1.4 | 0.6 |
| | ATTG---------------C | 0.3 | 0.7 | 1.7 | 0.9 | 0.7 |
| | ----------------ATGC | 1.7 | 1.6 | 0.9 | 1.4 | 0.4 |
| 151 | ATTGATGCTA-----GATGC | 0.5 | 0.8 | 1.6 | 1.0 | 0.6 |
| 152 | ----------TTCAGGATGC | 0.7 | 1.2 | 1.5 | 1.1 | 0.4 |
| 153 | ATTGATGCTAGTTCAGGATGC | 1.2 | 1.3 | 1.5 | 1.3 | 0.2 |
| 154 | ATTGATGCTA----GGATGC | 0.8 | 1.2 | 0.3 | 0.7 | 0.5 |
| 155 | ATTGATGCTATTTTCAGGAT GC | 1.2 | 0.6 | 0.8 | 0.9 | 0.3 |
| 156 | ATTGATGCTAT-------GC | 0.9 | 1.1 | 0.4 | 0.8 | 0.4 |
| 157 | ATTGATGCTA---------- | 1.0 | 0.9 | 0.8 | 0.9 | 0.1 |
| 158 | ATT--------TCAGGATGC | 0.8 | 0.9 | 1.0 | 0.9 | 0.1 |

^{a} On-target sequence centered on cleavage site, with 10 bp in either direction. For comparison, the portion of the gRNA target sequence aligning with the Reference on-target sequence is underlined and the PAM is indicated by parenthesis.
^{b} Deletions indicated by dashes (-); insertions indicated by bold

Table 6

On-Target Gene Edited Seuences > 1% Frequency in At Least One Gene Edited T Cell donor for the CBLB-7 gRNA
Reference on-target sequence^{a}: GGCAGCTTGCTTA (GGG) GGTC (SEQ ID NO: 293)

| SEQ ID NO: | Gene edited sequence^{b} | Donor 1 (%) | Donor 2 (%) | Donor 3 (%) | Mean (%) | Std. dev. (%) |
|---|---|---|---|---|---|---|
| 159 | GGCAGCTTGCCTTAGGGGGTC | 18.3 | 22.5 | 23.2 | 21.3 | 2.6 |
| | -------------------- | 8.4 | 6.4 | 5.9 | 6.9 | 1.3 |
| 160 | GGGAGCTTG--AGGGGGTC | 7.5 | 6.1 | 5.8 | 6.5 | 0.9 |
| 161 | GGCAGCTTGCTTTAGGGGGTC | 5.4 | 5.5 | 6.5 | 5.8 | 0.6 |
| 162 | GGCAGCTTGC-TAGGGGGTC | 3.9 | 5.0 | 3.2 | 4.0 | 0.9 |
| | G------------------- | 3.8 | 3.3 | 4.5 | 3.8 | 0.6 |
| 163 | GGCAGCTTGCTTTTAGGGGGTC | 1.7 | 1.5 | 2.8 | 2.0 | 0.7 |
| 164 | GGCAGCTTGCATTAGGGGGTC | 1.2 | 1.4 | 2.8 | 1.8 | 0.8 |
| 165 | GGCA----GCTTAGGGGGTC | 2.2 | 2.5 | 2.0 | 2.2 | 0.3 |
| 166 | GGCAGCTTG-TTAGGGGGTC | 1.6 | 2.1 | 1.3 | 1.7 | 0.4 |
| 167 | GGCAGCTTGCGTTAGGGGGTC | 1.1 | 2.0 | 1.5 | 1.5 | 0.4 |
| 168 | GGCAGCTTGC----GGGGTC | 1.9 | 1.7 | 0.8 | 1.5 | 0.6 |
| 169 | GGCAGCTT--TTAGGGGGTC | 0.5 | 1.9 | 1.3 | 1.2 | 0.7 |
| 170 | GGCAGCTTGC---GGGGGTC | 1.7 | 1.3 | 1.0 | 1.3 | 0.2 |
| 171 | GGC---------AGGGGGTC | 1.7 | 1.2 | 1.6 | 1.5 | 0.2 |
| | -----------------GGTC | 1.5 | 1.2 | 1.2 | 1.3 | 0.2 |
| | GG------------------ | 1.3 | 1.4 | 0.9 | 1.2 | 0.3 |
| 172 | GGCAGCTTGC------GGTC | 1.0 | 0.9 | 1.1 | 1.0 | 0.1 |
| | GGCAG----------------- | 0.9 | 1.1 | 0.7 | 0.9 | 0.2 |
| | GGCAGCT--------------- | 1.0 | 1.1 | 0.6 | 0.9 | 0.3 |
| | GGCAGCTTG-------------- | 1.0 | 0.8 | 0.5 | 0.8 | 0.2 |

^{a}On-target sequence centered on cleavage site, with 10bp in either direction. For comparison, the portion of the gRNA target sequence aligning with the Reference on-target sequence is underlined and the PAM is indicated by parenthesis.
^{b}Deletions indicated by dashes (-); insertions indicated by bold

Table 7

On-Target Gene Edited Sequences >1% Frequency in At Least One Gene Edited T Cell Donor for the CBLB-8 gRNA
Reference on-target sequence^{a}: AGTGCAACTTAAC (CGG) AAAA (SEQ ID NO: 294)

| SEQ ID NO: | Gene edited sequence^{b} SEQ ID NO: | Donor 1 (%) | Donor 2 (%) | Donor 3 (%) | Mean (%) | Std. dev. (%) |
|---|---|---|---|---|---|---|
| 173 | AGTGC-----AACCGGAAAA | 37.4 | | 32.2 | 34.8 | 3.7 |
| 174 | AGTGCAACTTTAACCGGAAAA | 6.3 | | 6.3 | 6.3 | 0.0 |
| 175 | AGTGCAACTT-ACCGGAAAA | 3.9 | | 3.6 | 3.8 | 0.2 |
| 176 | AGTGCAACTTAAACCGGAAAA | 3.7 | | 3.8 | 3.8 | 0.1 |
| | AG------------------- | 2.3 | | 1.7 | 2.0 | 0.4 |
| 177 | AGTGCAAC-TAACCGGAAAA | 1.9 | | 1.7 | 1.8 | 0.1 |
| 178 | ----------AACCGGAAAA | 1.8 | | 1.4 | 1.6 | 0.2 |
| | -------------------- | 1.7 | | 1.6 | 1.6 | 0.1 |
| 179 | AGTG------AACCGGAAAA | 0.8 | | 1.4 | 1.1 | 0.4 |
| 180 | AGTGCAAC--AACCGGAAAA | 1.4 | | 1.3 | 1.4 | 0.0 |
| | ---------------CGGAAAA | 1.0 | | 1.3 | 1.1 | 0.2 |
| 181 | AGTGCAA---AACCGGAAAA | 1.0 | | 0.6 | 0.8 | 0.3 |

^{a} On-target sequence centered on cleavage site, with 10 bp in either direction. For comparison, the portion of the gRNA target sequence aligning with the Reference-on-target sequence is underlined and the PAM is indicated by parenthesis.
^{b} Deletions indicated by dashes (-); insertions indicated by bold

TABLE 8

On-Target Gene Edited Sequences > 1% Frequency in At Least One Gene Edited T Cell Donor for the CBLB-12 gRNA
Reference on-target sequence^{a}: CGATTGGGACAGT (GGG) CCAT (SEQ ID NO: 295 )

| SEQ ID NO: | Gene edited sequence^{b} SEQ ID NO: | Donor 1 (%) | Donor 2 (%) | Donor 3 (%) | Mean (%) | Std. dev. (%) |
|---|---|---|---|---|---|---|
| 182 | CGATTGGG-------CCAT | 22.8 | 23.9 | 22.7 | 23.1 | 0.7 |
| | CGATTGG------------- | 7.7 | 9.2 | 8.6 | 8.5 | 0.8 |
| 183 | CGATTGGGA--GTGGGCCAT | 6.2 | 6.7 | 6.8 | 6.6 | 0.3 |
| 184 | CGA--------GTGGGCCAT | 5.2 | 4.6 | 3.6 | 4.5 | 0.8 |
| 185 | CGATTGGGACCAGTGGGCC AT | 4.5 | 4.7 | 4.1 | 4.4 | 0.3 |
| 186 | CGATTGGGACAAGTGGGCC AT | 2.7 | 3.4 | 4.6 | 3.5 | 0.9 |
| 187 | CGATTGGGA-GTGGGCCAT | 4.5 | 3.8 | 4.4 | 4.2 | 0.4 |
| 188 | CGATTGG---AGTGGGCCAT | 4.2 | 3.3 | 2.7 | 3.4 | 0.8 |
| 189 | CGATTGG---------CCAT | 1.4 | 1.1 | 2.2 | 1.6 | 0.6 |
| 190 | CGATTG----AGTGGGCCAT | 2.0 | 1.6 | 2.1 | 1.9 | 0.2 |
| 191 | CGATTGGGAC-------CAT | 1.9 | 2.1 | 2.0 | 2.0 | 0.1 |
| 192 | CGATTGGGACTAGTGGGCC AT | 1.3 | 1.9 | 1.2 | 1.5 | 0.4 |
| 193 | CGATTGGGAC-TGGGCCAT | 1.7 | 1.8 | 1.6 | 1.7 | 0.1 |
| | CGATTGGG------------ | 1.0 | 1.7 | 0.9 | 1.2 | 0.4 |
| 194 | CGATTGGGACA--------T | 1.4 | 1.0 | 1.4 | 1.3 | 0.2 |
| 195 | CGATTGGGAC--TGGGCCAT | 1.3 | 1.0 | 1.1 | 1.1 | 0.1 |
| 196 | CGATTGGGAC---------- | 0.8 | 1.2 | 0.9 | 1.0 | 0.2 |
| | -------------------- | 1.1 | 0.7 | 1.2 | 1.0 | 0.3 |
| | C----------------CAT | 1.0 | 0.7 | 1.2 | 1.0 | 0.2 |
| 197 | ----------AGTGGGCCAT | 0.8 | 1.1 | 0.5 | 0.8 | 0.3 |
| 198 | CGATT-----AGTGGGCCAT | 0.4 | 1.0 | 0.3 | 0.6 | 0.4 |

^{a} On-target sequence centered on cleavage site, with 10 bp in either direction. For comparison, the portion of the gRNA target sequence aligning with the Reference on-target sequence is underlined and the PAM is indicated by parenthesis.
^{b} Deletions indicated by dashes (-); insertions indicated by bold

TABLE 9

On-Target Gene Edited Sequences > 1% Frequency in At Least One Gene Edited T Cell Donor for the CBLB-13 gRNA
Reference on-target sequence^{a}: TTGCCTTACGGCA (TGG) CAGG (SEQ ID NO: 296)

| SEQ ID NO: | Gene edited sequence^{b} SEQ ID NO: | Donor 1 (%) | Donor 2 (%) | Donor 3 (%) | Mean (%) | Std. dev. (%) |
|---|---|---|---|---|---|---|
| 199 | TTGCCTTACGGGCATGGCAGG | 14.5 | 15.6 | 17.5 | 15.8 | 1.5 |
| 200 | TTGCCTTACG-CATGGCAGG | 9.3 | 11.8 | 12.2 | 11.1 | 1.6 |
| 201 | TTGC--------ATGGCAGG | 6.5 | 5.3 | 5.4 | 5.7 | 0.7 |
| 202 | TTGCCTTA--GCATGGCAGG | 6.1 | 5.5 | 3.9 | 5.1 | 1.2 |
| 203 | TTGCCTTACGCGGCATGGCA GG | 5.3 | 5.8 | 5.8 | 5.6 | 0.3 |
| | TTGC-------------AGG | 3.3 | 3.9 | 3.4 | 3.5 | 0.3 |
| | -------------------- | 2.6 | 2.9 | 1.5 | 2.3 | 0.7 |
| 204 | TTGCCTT--GGCATGGCAGG | 1.1 | 0.8 | 2.1 | 1.4 | 0.7 |
| 205 | TTGCCTTACG---------- | 1.9 | 2.1 | 1.8 | 1.9 | 0.2 |
| | TTG------------GCAGG | 2.1 | 2.1 | 1.6 | 1.9 | 0.3 |
| 206 | TTG-------GCATGGCAGG | 1.8 | 1.4 | 0.7 | 1.3 | 0.6 |
| 207 | TTGCCTTA-GGCATGGCAGG | 1.0 | 0.8 | 1.8 | 1.2 | 0.5 |
| 208 | TTGCCT----GCATGGCAGG | 1.4 | 1.6 | 1.4 | 1.4 | 0.1 |
| 209 | ----------GCATGGCAGG | 1.5 | 1.4 | 0.9 | 1.3 | 0.3 |
| | TTGCCTTA------------ | 1.4 | 1.1 | 1.5 | 1.3 | 0.2 |
| 210 | TTGCCTT---GCATGGCAGG | 1.4 | 1.0 | 1.0 | 1.1 | 0.2 |
| | ------------------GG | 1.3 | 0.8 | 0.7 | 0.9 | 0.3 |
| 211 | ---------GGCATGGCAGG | 1.0 | 1.3 | 1.0 | 1.1 | 0.1 |
| 212 | TTGCCTTACG--ATGGCAGG | 1.2 | 1.3 | 0.8 | 1.1 | 0.3 |
| 213 | TTGC-----GGCATGGCAGG | 0.5 | 0.9 | 1.2 | 0.9 | 0.4 |
| | TTGC---------------- | 1.1 | 0.8 | 0.9 | 0.9 | 0.1 |
| 214 | TTGCCTTAC---ATGGCAGG | 0.9 | 0.8 | 1.1 | 0.9 | 0.2 |
| | -------------TGGCAGG | 1.0 | 0.5 | 0.7 | 0.8 | 0.3 |
| 215 | TTGCCTT-------GGCAGG | 1.0 | 0.4 | 0.5 | 0.6 | 0.3 |

^{a} On-target sequence centered on cleavage site, with 10 bp in either direction. For comparison, the portion of the gRNA target sequence aligning with the Reference on-target sequence is underlined and the PAM is indicated by parenthesis.
^{b} Deletions indicated by dashes (-); insertions indicated by bold

TABLE 10

On-Target Gene Edited Sequences > 1% Frequency in At Least One Gene Edited T Cell Donor for the CBLB-15 gRNA
Reference on-target sequence ^{a}: AAACACATCCCGA (GGG) CACC (SEQ ID NO: 297 )

| SEQ ID NO: | Gene edited sequence^{b} SEQ ID NO: | Donor 1 (%) | Donor 2 (%) | Donor 3 (%) | Mean (%) | Std. dev. (%) |
|---|---|---|---|---|---|---|
| 216 | AAACACAT-CCGAGGGCACC | 19.2 | 21.2 | 21.5 | 20.6 | 1.2 |
| 217 | AAACACATCCCCGAGGGCACC | 12.0 | 12.0 | 13.0 | 12.4 | 0.6 |
| | AAA-------------CACC | 8.7 | 7.0 | 8.4 | 8.0 | 0.9 |
| | AAACA--------------C | 5.7 | 4.9 | 4.6 | 5.1 | 0.6 |
| | -------------------- | 3.5 | 5.2 | 4.7 | 4.5 | 0.9 |
| | ------------------CC | 3.7 | 4.1 | 3.6 | 3.8 | 0.3 |
| | AAACA-----------CACC | 2.5 | 2.5 | 2.0 | 2.3 | 0.3 |
| 218 | AAACACAT--CGAGGGCACC | 2.1 | 2.4 | 1.9 | 2.2 | 0.3 |
| 219 | AAACACAT----------CC | 2.1 | 1.2 | 0.9 | 1.4 | 0.6 |
| | ----------------CACC | 1.5 | 2.0 | 1.3 | 1.6 | 0.4 |
| | ---------------GCACC | 1.4 | 1.9 | 1.1 | 1.5 | 0.4 |
| | AAA----------------C | 1.3 | 1.6 | 1.5 | 1.4 | 0.2 |
| | -------------------C | 0.7 | 1.4 | 1.1 | 1.1 | 0.3 |
| | AA-------------GCACC | 1.3 | 0.3 | 0.8 | 0.8 | 0.5 |
| 220 | AAACA----CCGAGGGCACC | 1.3 | 1.0 | 1.2 | 1.1 | 0.1 |
| 221 | AAACA-----CGAGGGCACC | 0.6 | 1.0 | 1.2 | 1.0 | 0.3 |
| | ------------AGGGCACC | 1.2 | 1.0 | 1.2 | 1.2 | 0.1 |
| 222 | ----------CGAGGGCACC | 0.7 | 1.2 | 0.8 | 0.9 | 0.2 |
| | AA --------------- ACC | 0.8 | 0.8 | 1.1 | 0.9 | 0.2 |

^{a} On-target sequence centered on cleavage site, with 10 bp in either direction. For comparison, the portion of the gRNA target sequence aligning with the Reference on-target sequence is underlined and the PAM is indicated by parenthesis.
^{b} Deletions indicated by dashes (-); insertions indicated by bold

TABLE 11

On-Target Gene Edited Sequences > 1% Frequency in At Least One Gene Edited T Cell Donor for the CBLB-16 gRNA
Reference on-target sequence^{a}: CAAACACATCCCG (AGG) GCAC (SEQ ID NO: 298)

| SEQ ID NO: | Gene edited sequence^{b} SEQ ID NO: | Donor 1 (%) | Donor 2 (%) | Donor 3 (%) | Mean (%) | Std. dev. (%) |
|---|---|---|---|---|---|---|
| 256 | CAAACACAT-CCGAGGGCAC | 10.5 | 13.0 | 11.3 | 11.6 | 1.3 |
| | CAAA-------------CAC | 5.6 | 4.3 | 3.6 | 4.5 | 1.0 |
| 223 | CAAACACA-CCCGAGGGCAC | 1.9 | 2.5 | 4.5 | 3.0 | 1.3 |
| 224 | CAAACA---CCCGAGGGCAC | 2.6 | 3.1 | 4.2 | 3.3 | 0.8 |
| 225 | --------TCCCGAGGGCAC | 4.0 | 4.0 | 3.6 | 3.9 | 0.2 |
| 226 | CAAACACATCCCCGAGGGCAC | 3.9 | 2.9 | 3.2 | 3.4 | 0.5 |
| | -------------------- | 2.8 | 3.4 | 2.5 | 2.9 | 0.5 |
| | CAAACA-------------- | 3.1 | 2.9 | 3.3 | 3.1 | 0.2 |
| 227 | CAAACACA--CCGAGGGCAC | 1.5 | 2.7 | 1.4 | 1.9 | 0.8 |
| | -------------------C | 2.0 | 2.1 | 2.5 | 2.2 | 0.3 |
| 228 | ----------CCGAGGGCAC | 2.4 | 1.9 | 1.6 | 2.0 | 0.4 |
| 229 | CAAACACAT--CGAGGGCAC | 1.7 | 2.2 | 1.4 | 1.8 | 0.4 |
| 230 | CAAACA----CCGAGGGCAC | 2.0 | 1.5 | 1.4 | 1.6 | 0.3 |
| | -------------AGGGCAC | 1.5 | 1.2 | 1.0 | 1.2 | 0.2 |
| | CAAACA-----------CAC | 1.3 | 1.3 | 1.1 | 1.2 | 0.1 |
| 231 | ---------CCCGAGGGCAC | 1.3 | 1.0 | 0.9 | 1.0 | 0.2 |
| | CAAA---------------- | 0.7 | 1.2 | 0.6 | 0.8 | 0.3 |
| | ------------GAGGGCAC | 0.5 | 1.1 | 0.7 | 0.8 | 0.3 |

^{a} On-target sequence centered on cleavage site, with 10 bp in either direction. For comparison, the portion of the gRNA target sequence aligning with the Reference on-target sequence is underlined and the PAM is indicated by parenthesis.
^{b} Deletions indicated by dashes (-); insertions indicated by bold

TABLE 12

Exemplary AAV Donor Template Sequences

| Name | Sequence | SEQ ID NO: |
|---|---|---|
| Left ITR (5′ ITR) | TTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAACTCCATCACTAGGGGTTCCT | 232 |
| Left ITR (5′ ITR) (alternate) | CCTGCAGGCAGCTGCGCGCTCGCTCGCTCACTGAGGCCGCCCGGGCGTCGGGCGACCTTTGGTCGCCCGGCCTCAGTGAGCGAGCGAGCGCGCAGAGAGGGAGTGGCCAACTCCATCACTAGGGGTTCCT | 233 |
| Right ITR (3’ ITR) | AGGAACCCCTAGTGATGGAGTTGGCCACTCCCTCTCTGCGCGCTCGCTCGCTCACTGAGGCCGCCCGGGCAAAGCCCGGGCGTCGGGCGACCTTTGGTCGCCCGGCCTCAGTGAGCGAGCGAGCGCGCAGAGAGGGAGTGGCCAA | 234 |
| Right ITR (3′ ITR) (alternate) | AGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGCTGCCTGCAGG | 235 |
| TRAC- LHA (800bp) | GAGATGTAAGGAGCTGCTGTGACTTGCTCAAGGCCTTATATCGAGTAAACGGTAGTGCTGGGGCTTAGACGCAGGTGTTCTGATTTATAGTTCAAAACCTCTATCAATGAGAGAGCAATCTCCTGGTAATGTGATAGATTTCCCAACTTAATGCCAACATACCATAAACCTCCCATTCTGCTAATGCCCAGCCTAAGTTGGGGAGACCACTCCAGATTCCAAGATGTACAGTTTGCTTTGCTGGGCCTTTTTCCCATGCCTGCCTTTACTCTGCCAGAGTTATATTGCTGGGGTTTTGAAGAAGATCCTATTAAATAAAAGAATAAGCAGTATTATTAAGTAGCCCTGCATTTCAGGTTTCCTTGAGTGGCAGGCCAGGCCTGGCCGTGAACGTTCACTGAAATCATGGCCTCTTGGCCAAGATTGATAGCTTGTGCCTGTCCCTGAGTCCCAGTCCATCACGAGCAGCTGGTTTCTAAGATGCTATTTCCCGTATAAAGCATGAGACCGTGACTTGCCAGCCCCACAGAGCCCCGCCCTTGTCCATCACTGGCATCTGGACTCCAGCCTGGGTTGGGGCAAAGAGGGAAATGAGATCATGTCCTAACCCTGATCCTCTTGTCCCACAGATATCCAGAACCCTGACCCTGCCGTGTACCAGCTGAGAGACTCTAAATCCAGTGACAAGTCTGTCTGCCTATTCACCGATTTTGATTCTCAAACAAATGTGTCACAAAGTAAGGATTCTGATGTGTATATCACAGACAAAACTGTGCTAGACATGAGGTCTATGGACTTCA | 236 |
| TRAC- RHA (800bp) | TGGAGCAACAAATCTGACTTTGCATGTGCAAACGCCTTCAACAACA GCATTATTCCAGAAGACACCTTCTTCCCCAGCCCAGGTAAGGGCAGCTTTGGTGCCTTCGCAGGCTGTTTCCTTGCTTCAGGAATGGCCAGGTTCTGCCCAGAGCTCTGGTCAATGATGTCTAAAACTCCTCTGATTGGTGGTCTCGGCCTTATCCATTGCCACCAAAACCCTCTTTTTACTAAGAAACAGTGAGCCTTGTTCTGGCAGTCCAGAGAATGACACGGGAAAAAAGCAGATGAAGAGAAGGTGGCAGGAGAGGGCACGTGGCCCAGCCTCAGTCTCTCCAACTGAGTTCCTGCCTGCCTGCCTTTGCTCAGACTGTTTGCCCCTTACTGCTCTTCTAGGCCTCATTCTAAGCCCCTTCTCCAAGTTGCCTCTCCTTATTTCTCCCTGTCTGCCAAAAAATCTTTCCCAGCTCACTAAGTCAGTCTCACGCAGTCACTCATTAACCCACCAATCACTGATTGTGCCGGCACATGAATGCACCAGGTGTTGAAGTGGAGGAATTAAAAAGTCAGATGAGGGGTGTGCCCAGAGGAAGCACCATTCTAGTTGGGGGAGCCCATCTGTCAGCTGGGAAAAGTCCAAATAACTTCAGATTGGAATGTGTTTTAACTCAGGGTTGAGAAAACAGCTACCTTCAGGACAAAAGTCAGGGAAGGGCTCTCTGAAGAAATGCTACTTGAAGATACCAGCCCTACCAAGGGCAGGGAGAGGACCCTATAGAGGCCTGGGACAGGAGCTCAATGAGAAAGG | 237 |
| EF1α | GGCTCCGGTGCCCGTCAGTGGGCAGAGCGCACATCGCCCACAGTCCCCGAGAAGTTGGGGGGAGGGGTCGGCAATTGAACCGGTGCCTAGAGAAGGTGGCGCGGGGTAAACTGGGAAAGTGATGTCGTGTACTGGCTCCGCCTTTTTCCCGAGGGTGGGGGAGAACCGTATATAAGTGCAGTAGTCGCCGTGAACGTTCTTTTTCGCAACGGGTTTGCCGCCAGAACACAGGTAAGTGCCGTGTGTGGTTCCCGCGGGCCTGGCCTCTTTACGGGTTATGGCCCTTGCGTGCCTTGAATTACTTCCACTGGCTGCAGTACGTGATTCTTGATCCCGAGCTTCGGGTTGGAAGTGGGTGGGAGAGTTCGAGGCCTTGCGCTTAAGGAGCCCCTTCGCCTCGTGCTTGAGTTGAGGCCTGGCCTGGGCGCTGGGGCCGCCGCGTGCGAATCTGGTGGCACCTTCGCGCCTGTCTCGCTGCTTTCGATAAGTCTCTAGCCATTTAAAATTTTTGATGACCTGCTGCGACGCTTTTTTTCTGGCAAGATAGTCTTGTAAATGCGGGCCAAGATCTGCACACTGGTATTTCGGTTTTTGGGGCCGCGGGCGGCGACGGGGCCCGTGCGTCCCAGCGCACATGTTCGGCGAGGCGGGGCCTGCGAGCGCGGCCACCGAGAATCGGACGGGGGTAGTCTCAAGCTGGCCGGCCTGCTCTGGTGCCTGGCCTCGCGCCGCCGTGTATCGCCCCGCCCTGGGCGGCAAGGCTGGCCCGGTCGGCACCAGTTGCGTGAGCGGAAAGATGGCCGCTTCCCGGCCCTGCTGCAGGGAGCTCAAAATGGAGGACGCGGCGCTCGGGAGAGCGGGCGGGTGAGTCACCCACACAAAGGAAAAGGGCCTTTCCGTCCTCAGCCGTCGCTTCATGTGACTCCACGGAGTACCGGGCGCCGTCCAGGCACCTCGATTAGTTCTCGAGCTTTTGGAGTACGTCGTCTTTAGGTTGGGGGGAGGGGTTTTATGCGATGGAGTTTCCCCACACTGAGTGGGTGGAGACTGAAGTTAGGCCAGCTTGGCACTTGATGTAATTCTCCTTGGAATTTGCCCTTTTTGAGTTTGGATCTTGGTTCATTCTCAAGCCTCAGACAGTGGTTCAAAGTTTTTTTCTTCCATTTCAGGTGTCGTGA | 238 |
| Anti-CD70 CAR LHA toRHA (CD70B scFv with 41BB) | GAGATGTAAGGAGCTGCTGTGACTTGCTCAAGGCCTTATATCGAGTAAACGGTAGTGCTGGGGCTTAGACGCAGGTGTTCTGATTTATAGTTCAAAACCTCTATCAATGAGAGAGCAATCTCCTGGTAATGTGATAGATTTCCCAACTTAATGCCAACATACCATAAACCTCCCATTCTGCTAATGCCCAGCCTAAGTTGGGGAGACCACTCCAGATTCCAAGATGTACAGTTTGCTTTGCTGGGCCTTTTTCCCATGCCTGCCTTTACTCTGCCAGAGTTATATTGCTGGGGTTTTGAAGAAGATCCTATTAAATAAAAGAATAAGCAGTATTATTAAGTAGCCCTGCATTTCAGGTTTCCTTGAGTGGCAGGCCAGGCCTGGCCGTGAACGTTCACTGAAATCATGGCCTCTTGGCCAAGATTGATAGCTTGTGCCTGTCCCTGAGTCCCAGTCCATCACGAGCAGCTGGTTTCTAAGATGCTATTTCCCGTATAAAGCATGAGACCGTGACTTGCCAGCCCCACAGAGCCCCGCCCTTGTCCATCACTGGCATCTGGACTCCAGCCTGGGTTGGGGCAAAGAGGGAAATGAGATCATGTCCTAACCCTGATCCTCTTGTCCCACAGATATCCAGAACCCTGACCCTGCCGTGTACCAGCTGAGAGACTCTAAATCCAGTGACAAGTCTGTCTGCCTATTCACCGATTTTGATTCTCAAACAAATGTGTCACAAAGTAAGGATTCTGATGTGTATATCACAGACAAAACTGTGCTAGACATGAGGTCTATGGACTTCAGGCTCCGGTGCCCGTCAGTGGGCAGAGCGCACATCGCCCACAGTCCCCGAGAAGTTGGGGGGAGGGGTCGGCAATTGAACCGGTGCCTAGAGAAGGTGGCGCGGGGTAAACTGGGAAAGTGATGTCGTGTACTGGCTCCGCCTTTTTCCCGAGGGTGGGGGAGAACCGTATATAAGTGCAGTAGTCGCCGTGAACGTTCTTTTTCGCAACGGGTTTGCCGCCAGAACACAGGTAAGTGCCGTGTGTGGTTCCCGCGGGCCTGGCCTCTTTACGGGTTATGGCCCTTGCGTGCCTTGAATTACTTCCACTGGCTGCAGTACGTGATTCTTGATCCCGAGCTTCGGGTTGGAAGTGGGTGGGAGAGTTCGAGGCCTTGCGCTTAAGGAGCCCCTTCGCCTCGTGCTTGAGTTGAGGCCTGGCCTGGGCGCTGGGGCCGCCGCGTGCGAATCTGGTGGCACCTTCGCGCCTGTCTCGCTGCTTTCGATAAGTCTCTAGCCATTTAAAATTTTTGATGACCTGCTGCGACGCTTTTTTTCTGGCAAGATAGTCTTGTAAATGCGGGCCAAGATCTGCACACTGGTATTTCGGTTTTTGGGGCCGCGGGCGGCGACGGGGCCCGTGCGTCCCAGCGCACATGTTCGGCGAGGCGGGGCCTGCGAGCGCGGCCACCGAGAATCGGACGGGGGTAGTCTCAAGCTGGCCGGCCTGCTCTGGTGCCTGGCCTCGCGCCGCCGTGTATCGCCCCGCCCTGGGCGGCAAGGCTGGCCCGGTCGGCACCAGTTGCGTGAGCGGAAAGATGGCCGCTTCCCGGCCCTGCTGCAGGGAGCTCAAAATGGAGGACGCGGCGCTCGGGAGAGCGGGCGGGTGAGTCACCCACACAAAGGAAAAGGGCCTTTCCGTCCTCAGCCGTCGCTTCATGTGACTCCACGGAGTACCGGGCGCCGTCCAGGCACCTCGATTAGTTCTCGAGCTTTTGGAGTACGTCGTCTTTAGGTTGGGGGGAGGGGTTTTATGCGATGGAGTTTCCCCACACTGAGTGGGTGGAGACTGAAGTTAGGCCAGCTTGGCACTTGATGTAATTCTCCTTGGAATTTGCCCTTTTTGAGTTTGGATCTTGGTTCATTCTCAAGCCTCAGACAGTGGTTCAAAGTTTTTTTCTTCCATTTCAGGTGTCGTGACCACCATGGCGCTTCCGGTGACAGCACTGCTCCTCCCCTTGGCGCTGTTGCTCCACGCAGCAAGGCCGCAGGTCCAGTTGGTGCAAAGCGGGGCGGAGGTGAAAAAACCCGGCGCTTCCGTGAAGGTGTCCTGTAAGGCGTCCGGTTATACGTTCACGAACTACGGGATGAATTGGGTTCGCCAAGCGCCGGGGCAGGGACTGAAATGGATGGGGTGGATAAATACCTACACCGGCGAACCTACATACGCCGACGCTTTTAAAGGGCGAGTCACTATGACGCGCGATACCAGCATATCCACCGCATACATGGAGCTGTCCCGACTCCGGTCAGACGACACGGCTGTCTACTATTGTGCTCGGGACTATGGCGATTATGGCATGGACTACTGGGGTCAGGGTACGACTGTAACAGTTAGTAGTGGTGGAGGCGGCAGTGGCGGGGGGGGAAGCGGAGGAGGGGGTTCTGGTGACATAGTTATGACCCAATCCCCAGATAGTTTGGCGGTTTCTCTGGGCGAGAGGGCAACGA | 239 |
| | TTAATTGTCGCGCATCAAAGAGCGTTTCAACGAGCGGATATTCTTTTATGCATTGGTACCAGCAAAAACCCGGACAACCGCCGAAGCTGCTGATCTACTTGGCTTCAAATCTTGAGTCTGGGGTGCCGGACCGATTTTCTGGTAGTGGAAGCGGAACTGACTTTACGCTCACGATCAGTTCACTGCAGGCTGAGGATGTAGCGGTCTATTATTGCCAGCACAGTAGAGAAGTCCCCTGGACCTTCGGTCAAGGCACGAAAGTAGAAATTAAAAGTGCTGCTGCCTTTGTCCCGGTATTTCTCCCAGCCAAACCGACCACGACTCCCGCCCCGCGCCCTCCGACACCCGCTCCCACCATCGCCTCTCAACCTCTTAGTCTTCGCCCCGAGGCATGCCGACCCGCCGCCGGGGGTGCTGTTCATACGAGGGGCTTGGACTTCGCTTGTGATATTTACATTTGGGCTCCGTTGGCGGGTACGTGCGGCGTCCTTTTGTTGTCACTCGTTATTACTTTGTATTGTAATCACAGGAATCGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGCGAGTGAAGTTTTCCCGAAGCGCAGACGCTCCGGCATATCAGCAAGGACAGAATCAGCTGTATAACGAACTGAATTTGGGACGCCGCGAGGAGTATGACGTGCTTGATAAACGCCGGGGGAGAGACCCGGAAATGGGGGGTAAACCCCGAAGAAAGAATCCCCAAGAAGGACTCTACAATGAACTCCAGAAGGATAAGATGGCGGAGGCCTACTCAGAAATAGGTATGAAGGGCGAACGACGACGGGGAAAAGGTCACGATGGCCTCTACCAAGGGTTGAGTACGGCAACCAAAGATACGTACGATGCACTGCATATGCAGGCCCTGCCTCCCAGATAATAATAAAATCGCTATCCATCGAAGATGGATGTGTGTTGGTTTTTTGTGTGTGGAGCAACAAATCTGACTTTGCATGTGCAAACGCCTTCAACAACAGCATTATTCCAGAAGACACCTTCTTCCCCAGCCCAGGTAAGGGCAGCTTTGGTGCCTTCGCAGGCTGTTTCCTTGCTTCAGGAATGGCCAGGTTCTGCCCAGAGCTCTGGTCAATGATGTCTAAAACTCCTCTGATTGGTGGTCTCGGCCTTATCCATTGCCACCAAAACCCTCTTTTTACTAAGAAACAGTGAGCCTTGTTCTGGCAGTCCAGAGAATGACACGGGAAAAAAGCAGATGAAGAGAAGGTGGCAGGAGAGGGCACGTGGCCCAGCCTCAGTCTCTCCAACTGAGTTCCTGCCTGCCTGCCTTTGCTCAGACTGTTTGCCCCTTACTGCTCTTCTAGGCCTCATTCTAAGCCCCTTCTCCAAGTTGCCTCTCCTTATTTCTCCCTGTCTGCCAAAAAATCTTTCCCAGCTCACTAAGTCAGTCTCACGCAGTCACTCATTAACCCACCAATCACTGATTGTGCCGGCACATGAATGCACCAGGTGTTGAAGTGGAGGAATTAAAAAGTCAGATGAGGGGTGTGCCCAGAGGAAGCACCATTCTAGTTGGGGGAGCCCATCTGTCAGCTGGGAAAAGTCCAAATAACTTCAGATTGGAATGTGTTTTAACTCAGGGTTGAGAAAACAGCTACCTTCAGGACAAAAGTCAGGGAAGGGCTCTCTGAAGAAATGCTACTTGAAGATACCAGCCCTACCAAGGGCAGGGAGAGGACCCTATAGAGGCCTGGGACAGGAGCTCAATGAGAAAGG | |
| Anti-BCMA CAR RHA to LHA | GAGATGTAAGGAGCTGCTGTGACTTGCTCAAGGCCTTATATCGAGTAAACGGTAGTGCTGGGGCTTAGACGCAGGTGTTCTGATTTATAGTTCAAAACCTCTATCAATGAGAGAGCAATCTCCTGGTAATGTGATAGATTTCCCAACTTAATGCCAACATACCATAAACCTCCCATTCTGCTAATGCCCAGCCTAAGTTGGGGAGACCACTCCAGATTCCAAGATGTACAGTTTGCTTTGCTGGGCCTTTTTCCCATGCCTGCCTTTACTCTGCCAGAGTTATATTGCTGGGGTTTTGAAGAAGATCCTATTAAATAAAAGAATAAGCAGTATTATTAAGTAGCCCTGCATTTCAGGTTTCCTTGAGTGGCAGGCCAGGCCTGGCCGTGAACGTTCACTGAAATCATGGCCTCTTGGCCAAGATTGATAGCTTGTGCCTGTCCCTGAGTCCCAGTCCATCACGAGCAGCTGGTTTCTAAGATGCTATTTCCCGTATAAAGCATGAGACCGTGACTTGCCAGCCCCACAGAGCCCCGCCCTTGTCCATCACTGGCATCTGGACTCCAGCCTGGGTTGGGGCAAAGAGGGAAATGAGATCATGTCCTAACCCTGATCCTCTTGTCCCACAGATATCCAGAACCCTGACCCTGCCGTGTACCAGCTGAGAGACTCTAAATCCAGTGACAAGTCTGTCTGCCTATTCACCGATTTTGATTCTCAAACAAATGTGTCACAAAGTAAGGATTCTGATGTGTATATCACAGACAAAACTGTGCTAGACATGAGGTCTATGGACTTCAGGCTCCGGTGCCCGTCAGTGGGCAGAGCGCACATCGCCCACAGTCCCCGAGAAGTTGGGGGGAGGGGTCGGCAATTGAACCGGTGCCTAGAGAAGGTGGCGCGGGGTAAACTGGGAAAGTGATGTCGTGTACTGGCTCCGCCTTTTTCCCGAGGGTGGGGGAGAACCGTATATAAGTGCAGTAGTCGCCGTGAACGTTCTTTTTCGCAACGGGTTTGCCGCCAGAACACAGGTAAGTGCCGTGTGTGGTTCCCGCGGGCCTGGCCTCTTTACGGGTTATGGCCCTTGCGTGCCTTGAATTACTTCCACTGGCTGCAGTACGTGATTCTTGATCCCGAGCTTCGGGTTGGAAGTGGGTGGGAGAGTTCGAGGCCTTGCGCTTAAGGAGCCCCTTCGCCTCGTGCTTGAGTTGAGGCCTGGCCTGGGCGCTGGGGCCGCCGCGTGCGAATCTGGTGGCACCTTCGCGCCTGTCTCGCTGCTTTCGATAAGTCTCTAGCCATTTAAAATTTTTGATGACCTGCTGCGACGCTTTTTTTCTGGCAAGATAGTCTTGTAAATGCGGGCCAAGATCTGCACACTGGTATTTCGGTTTTTGGGGCCGCGGGCGGCGACGGGGCCCGTGCGTCCCAGCGCACATGTTCGGCGAGGCGGGGCCTGCGAGCGCGGCCACCGAGAATCGGACGGGGGTAGTCTCAAGCTGGCCGGCCTGCTCTGGTGCCTGGCCTCGCGCCGCCGTGTATCGCCCCGCCCTGGGCGGCAAGGCTGGCCCGGTCGGCACCAGTTGCGTGAGCGGAAAGATGGCCGCTTCCCGGCCCTGCTGCAGGGAGCTCAAAATGGAGGACGCGGCGCTCGGGAGAGCGGGCGGGTGAGTCACCCACACAAAGGAAAAGGGCCTTTCCGTCCTCAGCCGTCGCTTCATGTGACTCCACGGAGTACCGGGCGCCGTCCAGGCACCTCGATTAGTTCTCGAGCTTTTGGAGTACGTCGTCTTTAGGTTGGGGGGAGGGGTTTTATGCGATGGAGTTTCCCCACACTGAGTGGGTGGAGACTGAAGTTAGGCCAGCTTGGCACTTGATGTAATTCTCCTTGGAATTTGCCCTTTTTGAGTTTGGATCTTGGTTCATTCTCAAGCCTCAGACAGTGGTTCAAAGTTTTTTTCTTCCATTTCAGGTGTCGTGACCACCATGGCGCTTCCGGTGACAGCACTGCTCCTCCCCTTGGCGCTGTTGCTCCACGCAGCAAGGCCGCAGGTGCAGCTGGTGCAGAGCGGAGCCGAGCTCAAGAAGCCCGGAGCCTCCGTGAAGGTGAGCTGCAAGGCCAGCGGCAACACCCTGACCAACTACGTGATCCACTGGGTGAGACAAGCCCCCGGCCAAAGGCTGGAGTGGATGGGCTACA TCCTGCCCTACAACGACCTGACCAAGTACAGCCAGAAGTTCCAGGGCAGGGTGACCATCACCAGGGATAAGAGCGCCTCCACCGCCTATATGGAGCTGAGCAGCCTGAGGAGCGAGGACACCGCTGTGTACTACTGTACAAGGTGGGACTGGGACGGCTTCTTTGACCCCTGGGGCCAGGGCACAACAGTGACCGTCAGCAGCGGCGGCGGAGGCAGCGGCGGCGGCGGCAGCGGCGGAGGCGGAAGCGAAATCGTGATGACCCAGAGCCCCGCCACACTGAGCGTGAGCCCTGGCGAGAGGGCCAGCATCTCCTGCAGGGCTAGCCAAAGCCTGGTGCACAGCAACGGCAACACCCACCTGCACTGGTACCAGCAGAGACCCGGACAGGCTCCCAGGCTGCTGATCTACAGCGTGAGCAACAGGTTCTCCGAGGTGCCTGCCAGGTTTAGCGGCAGCGGAAGCGGCACCGACTTTACCCTGACCATCAGCAGCGTGGAGTCCGAGGACTTCGCCGTGTATTACTGCAGCCAGACCAGCCACATCCCTTACACCTTCGGCGGCGGCACCAAGCTGGAGATCAAAAGTGCTGCTGCCTTTGTCCCGGTATTTCTCCCAGCCAAACCGACCACGACTCCCGCCCCGCGCCCTCCGACACCCGCTCCCACCATCGCCTCTCAACCTCTTAGTCTTCGCCCCGAGGCATGCCGACCCGCCGCCGGGGGTGCTGTTCATACGAGGGGCTTGGACTTCGCTTGTGATATTTACATTTGGGCTCCGTTGGCGGGTACGTGCGGCGTCCTTTTGTTGTCACTCGTTATTACTTTGTATTGTAATCACAGGAATCGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGCGAGTGAAGTTTTCCCGAAGCGCAGACGCTCCGGCATATCAGCAAGGACAGAATCAGCTGTATAACGAACTGAATTTGGGACGCCGCGAGGAGTATGACGTGCTTGATAAACGCCGGGGGAGAGACCCGGAAATGGGGGGTAAACCCCGAAGAAAGAATCCCCAAGAAGGACTCTACAATGAACTCCAGAAGGATAAGATGGCGGAGGCCTACTCAGAAATAGGTATGAAGGGCGAACGACGACGGGGAAAAGGTCACGATGGCCTCTACCAAGGGTTGAGTACGGCAACCAAAGATACGTACGATGCACTGCATATGCA GGCCCTGCCTCCCAGATAATAATAAAATCGCTATCCATCGAAGATGGATGTGTGTTGGTTTTTTGTGTGTGGAGCAACAAATCTGACTTTGCATGTGCAAACGCCTTCAACAACAGCATTATTCCAGAAGACACCTTCTTCCCCAGCCCAGGTAAGGGCAGCTTTGGTGCCTTCGCAGGCTGTTTCCTTGCTTCAGGAATGGCCAGGTTCTGCCCAGAGCTCTGGTCAATGATGTCTAAAACTCCTCTGATTGGTGGTCTCGGCCTTATCCATTGCCACCAAAACCCTCTTTTTACTAAGAAACAGTGAGCCTTGTTCTGGCAGTCCAGAGAATGACACGGGAAAAAAGCAGATGAAGAGAAGGTGGCAGGAGAGGGCACGTGGCCCAGCCTCAGTCTCTCCAACTGAGTTCCTGCCTGCCTGCCTTTGCTCAGACTGTTTGCCCCTTACTGCTCTTCTAGGCCTCATTCTAAGCCCCTTCTCCAAGTTGCCTCTCCTTATTTCTCCCTGTCTGCCAAAAAATCTTTCCCAGCTCACTAAGTCAGTCTCACGCAGTCACTCATTAACCCACCAATCACTGATTGTGCCGGCACATGAATGCACCAGGTGTTGAAGTGGAGGAATTAAAAAGTCAGATGAGGGGTGTGCCCAGAGGAAGCACCATTCTAGTTGGGGGAGCCCATCTGTCAGCTGGGAAAAGTCCAAATAACTTCAGATTGGAATGTGTTTTAACTCAGGGTTGAGAAAACAGCTACCTTCAGGACAAAAGTCAGGGAAGGGCTCTCTGAAGAAATGCTACTTGAAGATACCAGCCCTACCAAGGGCAGGGAGAGGACCCTATAGAGGCCTGGGACAGGAGCTCAATGAGAAAGG | 240 |

TABLE 13

Chimeric Antigen Receptor Sequences

| SEQ ID NO | Description | Sequence |
|---|---|---|
| 241 | signal peptide | MLLLVTSLLLCELPHPAFLLIP |
| 242 | signal peptide | MALPVTALLLPLALLLHAARP |
| 243 | CD8a transmembrane domain | IYIWAPLAGTCGVLLLSLVITLY |
| 244 | 4-1BB nucleotide sequence | AAACGGGGCAGAAAGAAACTCCTGTATATATTCAAACAA CCATTTATGAGACCAGTACAAACTACTCAAGAGGAAGAT GGCTGTAGCTGCCGATTTCCAGAAGAAGAAGAAGGAGGATGTGAACTG |
| 245 | 4-1BB amino acid sequence | KRGRKKLLYIFKQPFMRPVQTTQEEDGCSCRFPEEEEGGCEL |
| 246 | CD28 nucleotide sequence | TCAAAGCGGAGTAGGTTGTTGCATTCCGATTACATGAATATGACTCCTCGCCGGCCTGGGCCGACAAGAAAACATTACCAACCCTATGCCCCCCCACGAGACTTCGCTGCGTACAGGTCC |
| 247 | CD28 amino acid sequence | SKRSRLLHSDYMNMTPRRPGPTRKHYQPYAPPRDFAAYRS |
| 248 | CD3-zeta nucleotide sequence | CGAGTGAAGTTTTCCCGAAGCGCAGACGCTCCGGCATATCAGCAAGGACAGAATCAGCTGTATAACGAACTGAATTTGGGACGCCGCGAGGAGTATGACGTGCTTGATAAACGCCGGGGGAGAGACCCGGAAATGGGGGGTAAACCCCGAAGAAAGAATCCCCAAGAAGGACTCTACAATGAACTCCAGAAGGATAAGATGGCGGAGGCCTACTCAGAAATAGGTATGAAGGGCGAACGACGACGGGGAAAAGGTCACGATGGCCTCTACCAAGGGTTGAGTACGGCAACCAAAGATACGTACGATGCACTGCATATGCAGGCCCTGCCTCCCAGA |
| 249 | CD3-zeta amino acid sequence | RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR |
| 250 | CD8a extracellular + CD8a transmembrane + 5′ Linker (underlined) | GCTGCTGCCTTTGTCCCGGTATTTCTCCCAGCCAAACCGACCACGACTCCCGCCCCGCGCCCTCCGACACCCGCTCCCACCATCGCCTCTCAACCTCTTAGTCTTCGCCCCGAGGCATGCCGACCCGCCGCCGGGGGTGCTGTTCATACGAGGGGCTTGGACTTCGCTTGTGATATTTACATTTGGGCTCCGTTGGCGGGTACGTGCGGCGTCCTTTTGTTGTCACTCGTTATTACTTTGTATTGTAATCACAGGAATCGC |
| 251 | CD8a extracellular +CD8a transmembrane (without linker) | TTTGTCCCGGTATTTCTCCCAGCCAAACCGACCACGACTC CCGCCCCGCGCCCTCCGACACCCGCTCCCACCATCGCCTCTCAACCTCTTAGTCTTCGCCCCGAGGCATGCCGACCCGCCGCCGGGGGTGCTGTTCATACGAGGGGCTTGGACTTCGCTTGTGATATTTACATTTGGGCTCCGTTGGCGGGTACGTGCGGCGTCCTTTTGTTGTCACTCGTTATTACTTTGTATTGTAATCACAGGAATCGC |
| 252 | CD8a extracellular + CD8a transmembrane | FVPVFLPAKPTTTPAPRPPTPAPTIASQPLSLRPEACRPAAGGAVHTRGLDFACDIYIWAPLAGTCGVLLLSLVITLYCNHRNR |
| 253 | CD70 VL CDR1 (Kabat) | RASKSVSTSGYSFMH |
| 254 | CD70 VL CDR1 (Chothia) | SKSVSTSGYSF |
| 255 | CD70 VL CDR2 (Kabat) | LASNLES |
| | CD70 VL CDR2 (Chothia) | LAS |
| 257 | CD70 VL CDR3 (Kabat) | QHSREVPWT |
| 258 | CD70 VL CDR3 (Chothia) | SREVPW |
| 259 | CD70 VH CDR1 (Kabat) | NYGMN |
| 260 | CD70 VH CDR1 (Chothia) | GYTFTNYGMN |
| 261 | CD70 VH CDR2 (Kabat) | WINTYTGEPTYADAFKG |
| 262 | CD70 VH CDR2 (Chothia) | NTYTGE |
| 263 | CD70 VH CDR3 (Kabat) | DYGDYGMDY |
| 264 | CD70 VH CDR3 (Chothia) | CARDYGDYGMDYWG |
| 265 | CD70 CAR amino acid sequence (CD70B scFv with 41BB) With signal peptide | MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR |
| 266 | CD70 CAR amino acid sequence (CD70B scFv with 41BB) Without signal peptide | QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRA SKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR |
| 267 | Anti-CD70A scFv nucleotide sequence | GATATAGTTATGACCCAATCACCCGATAGTCTTGCGGTAAGCCTGGGGGAGCGAGCAACAATAAACTGTCGGGCATCAAAATCCGTCAGTACAAGCGGGTATTCATTCATGCACTGGTATCAACAGAAACCCGGTCAGCCACCCAAGCTCCTGATTTATCTTGCGTCTAATCTTGAGTCCGGCGTCCCAGACCGGTTTTCCGGCTCCGGGAGCGGCACGGATTTTACTCTTACTATTTCTAGCCTTCAGGCCGAAGATGTGGCGGTATACTACTGCCAGCATTCAAGGGAAGTTCCTTGGACGTTCGGTCAGGGCACGAAAGTGGAAATTAAAGGCGGGGGGGGATCCGGCGGGGGAGGGTCTGGAGGAGGTGGCAGTGGTCAGGTCCAACTGGTGCAGTCCGGGGCAGAGGTAAAAAAACCCGGCGCGTCTGTTAAGGTTTCATGCAAGGCCAGTGGATATACTTTCACCAATTACGGAATGAACTGGGTGAGGCAGGCCCCTGGTCAAGGCCTGAAATGGATGGGATGGATAAACACGTACACCGGTGAACCTACCTATGCCGATGCCTTTAAGGGTCGGGTTACGATGACGAGAGACACCTCCATATCAACAGCCTACATGGAGCTCAGCAGATTGAGGAGTGACGATACGGCAGTCTATTACTGTGCAAGAGACTACGGCGATTATGGCATGGATTACTGGGGCCAGGGCACTACAGTAACCGTTTCCAGC |
| 268 | Anti-CD70A scFv amino acid sequence (linker underlined) | DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGGGGSGGGGSGGGGSGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS |
| 269 | Anti-CD70B scFv nucleotide sequence | CAGGTCCAGTTGGTGCAAAGCGGGGCGGAGGTGAAAAAACCCGGCGCTTCCGTGAAGGTGTCCTGTAAGGCGTCCGGTTATACGTTCACGAACTACGGGATGAATTGGGTTCGCCAAGCGCCGGGGCAGGGACTGAAATGGATGGGGTGGATAAATACCTACACCGGCGAACCTACATACGCCGACGCTTTTAAAGGGCGAGTCACTATGACGCGCGATACCAGCATATCCACCGCATACATGGAGCTGTCCCGACTCCGGTCAGACGACACGGCTGTCTACTATTGTGCTCGGGACTATGGCGATTATGGCATGGACTACTGGGGTCAGGGTACGACTGTAACAGTTAGTAGTGGTGGAGGCGGCAGTGGCGGGGGGGGAAGCGGAGGAGGGGGTTCTGGTGACATAGTTATGACCCAATCCCCAGATAGTTTGGCGGTTTCTCTGGGCGAGAGGGCAACGATTAATTGTCGCGCATCAAAGAGCGTTTCAACGAGCGGATATTCTTTTATGCATTGGTACCAGCAAAAACCCGGACAACCGCCGAAGCTGCTGATCTACTTGGCTTCAAATCTTGAGTCTGGGGTGCCGGACCGATTTTCTGGTAGTGGAAGCGGAACTGACTTTACGCTCACGATCAGTTCACTGCAGGCTGAGGATGTAGCGGTCTATTATTGCCAGCACAGTAGAGAAGTCCCCTGGACCTTCGGTCAAGGCACGAAAGTAGAAATTAAA |
| 270 | Anti-CD70B scFv amino acid sequence (linker underlined) | QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK |
| 271 | Anti-CD70 VH | QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS |
| 272 | Anti-CD70 VL | DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK |
| 273 | Anti-BCMA CAR nucleotide sequence | ATGGCGCTTCCGGTGACAGCACTGCTCCTCCCCTTGGCGCTGTTGCTCCACGCAGCAAGGCCGCAGGTGCAGCTGGTGCAGAGCGGAGCCGAGCTCAAGAAGCCCGGAGCCTCCGTGAAGGTGAGCTGCAAGGCCAGCGGCAACACCCTGACCAACTACGTGATCCACTGGGTGAGACAAGCCCCCGGCCAAAGGCTGGAGTGGATGGGCTACATCCTGCCCTACAACGACCTGACCAAGTACAGCCAGAAGTTCCAGGGCAGGGTGACCATCACCAGGGATAAGAGCGCCTCCACCGCCTATATGGAGCTGAGCAGCCTGAGGAGCGAGGACACCGCTGTGTACTACTGTACAAGGTGGGACTGGGACGGCTTCTTTGACCCCTGGGGCCAGGGCACAACAGTGACCGTCAGCAGCGGCGGCGGAGGCAGCGGCGGCGGCGGCAGCGGCGGAGGCGGAAGCGAAATCGTGATGACCCAGAGCCCCGCCACACTGAGCGTGAGCCCTGGCGAGAGGGCCAGCATCTCCTGCAGGGCTAGCCAAAGCCTGGTGCACAGCAACGGCAACACCCACCTGCACTGGTACCAGCAGAGACCCGGACAGGCTCCCAGGCTGCTGATCTACAGCGTGAGCAACAGGTTCTCCGAGGTGCCTGCCAGGTTTAGCGGCAGCGGAAGCGGCACCGACTTTACCCTGACCATCAGCAGCGTGGAGTCCGAGGACTTCGCCGTGTATTACTGCAGCCAGACCAGCCACATCCCTTACACCTTCGGCGGCGGCACCAAGCTGGAGATCAAAAGTGCTGCTGCCTTTGTCCCGGTATTTCTCCCAGCCAAACCGACCACGACTCCCGCCCCGCGCCCTCCGACACCCGCTCCCACCATCGCCTCTCAACCTCTTAGTCTTCGCCCCGAGGCATGCCGACCCGCCGCCGGGGG TGCTGTTCATACGAGGGGCTTGGACTTCGCTTGTGATATTTACATTTGGGCTCCGTTGGCGGGTACGTGCGGCGTCCTTTTGTTGTCACTCGTTATTACTTTGTATTGTAATCACAGGAATCGCAAACGGGGCAGAAAGAAACTCCTGTATATATTCAAACAACCATTTATGAGACCAGTACAAACTACTCAAGAGGA AGATGGCTGTAGCTGCCGATTTCCAGAAGAAGAAGAAGGAGGATGTGAACTGCGAGTGAAGTTTTCCCGAAGCGCAGACGCTCCGGCATATCAGCAAGGACAGAATCAGCTGTATAACGAACTGAATTTGGGACGCCGCGAGGAGTATGACGTGCTTGATAAACGCCGGGGGAGAGACCCGGAAATGGGGGGTAAACCCCGAAGAAAGAATCCCCAAGAAGGACTCTACAATGAACTCCAGAAGGATAAGATGGCGGAGGCCTACTCAGAAATAGGTATGAAGGGCGAACGACGACGGGGAAAAGGTCACGATGGCCTCTACCAAGGGTTGAGTACGGCAACCAAAGATACGTACGATGCACTGCATATGCAGGCCCTGCCTCCCAGA |
| 274 | Anti-BCMA CAR amino acid sequence With signal peptide | MALPVTALLLPLALLLHAARPQVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR |
| 275 | Anti-BCMA CAR amino acid sequence Without signal peptide | QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR |
| 276 | Anti-BCMA scFv nucleotide sequence | CAGGTGCAGCTGGTGCAGAGCGGAGCCGAGCTCAAGAAGCCCGGAGCCTCCGTGAAGGTGAGCTGCAAGGCCAGCGGCAACACCCTGACCAACTACGTGATCCACTGGGTGAGACAAGCCCCCGGCCAAAGGCTGGAGTGGATGGGCTACATCCTGCCCTACAACGACCTGACCAAGTACAGCCAGAAGTTCCAGGGCAGGGTGACCATCACCAGGGATAAGAGCGCCTCCACCGCCTATATGGAGCTGAGCAGCCTGAGGAGCGAGGACACCGCTGTGTACTACTGTACAAGGTGGGACTGGGACGGCTTCTTTGACCCCTGGGGCCAGGGCACAACAGTGACCGTCAGCAGCGGCGGCGGAGGCAGCGGCGGCGGCGGCAGCGGCGGAGGCGGAAGCGAAATCGTGATGACCCAGAGCCCCGCCACACTGAGCGTGAGCCCTGGCGAGAGGGCCAGCATCTCCTGCAGGGCTAGCCAAAGCCTGGTGCACAGCAACGGCAACACCCACCTGCACTGGTACCAGCAGAGACCCGGACAGGCTCCCAGGCTGCTGATCTACAGCGTGAGCAACAGGTTCTCCGAGGTGCCTGCCAGGTTTAGCGGCAGCGGAAGCGGCACCGACTTTACCCTGACCATCAGCAGCGTGGAGTCCGAGGACTTCGCCGTGTATTACTGCAGCCAGACCAGCCACATCCCTTACACCTTCGGCGGCGGCACCAAGCTGGAGATCAAA |
| 277 | Anti-BCMA scFv amino acid sequence (linker underlined) | QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK |
| 278 | Anti-BCMA VH | QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSS |
| 279 | Anti-BCMA VL | EIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK |
| 280 | BCMA VL CDR1 (Kabat & Chothia) | RASQSLVHSNGNTHLH |
| 281 | BCMA VL CDR2 (Kabat & Chothia) | SVSNRFS |
| 282 | BCMA VL CDR3 (Kabat) | SQTSHIPYT |
| 283 | BCMA VL CDR3 (Chothia) | SQTSHIPYT |
| 284 | BCMA VH CDR1 (Kabat) | NYVIH |
| 285 | BCMA VH CDR1 (Chothia) | GNTLTNY |
| 286 | BCMA VH CDR2 (Kabat) | YILPYNDLTKYSQKFQG |
| 287 | BCMA VH CDR2 (Chothia) | LPYNDL |
| 288 | BCMA VH CDR3 (Kabat) | WDWDGFFDP |
| 289 | BCMA VH CDR3 (Chothia) | WDWDGFFDP |

TABLE 20

sgRNA Sequences and Target Gene Sequences for Reg1

| Name | Unmodified Sequence | Modified Sequence | Target Sequences (PAM) |
|---|---|---|---|
| REG1-Z01 sgRNA | GGUCAUCGAUGGGAGCAACGguuuuagagcuagaaauagcaaguuaaaauaagg | G*G*U*CAUCGAUGGGAGCAACGguuuuagagcuagaaauagcaaguuaaaauaaggcuaguccgu | GGTCATCGATGGGAGCAACG (TGG) (SEQ ID NO: 339) |
| (EX2_T1) | cuaguccguuaucaacuu gaaaaaguggcaccgagu cggugcUUUU (SEQ ID NO: 299) | uaucaacuugaaaaaguggcac cgagucggugcU*U*U*U (SEQ ID NO: 300) | GGTCATCGATGGGAGCAACG (SEQ ID NO: 340) |
| REG1-Z01 sgRNA (EX2_T1) spacer | GGUCAUCGAUGGGAGCAACG (SEQ ID NO: 301) | G*G*U*CAUCGAUGGGAGCAAC G (SEQ ID NO: 302) | |
| REG1-Z02 sgRNA (EX2_T2) | CACCACCCCGCGGGACUAGAguuuuagagcuagaaauagcaaguuaaaauaaggcuaguccguuaucaacuugaaaaaguggcaccgagucggugcUUUU (SEQ ID NO: 303) | C*A*C*CACCCCGCGGGACUAGAguuuuagagcuagaaauagcaaguuaaaauaaggcuaguccguuaucaacuugaaaaaguggcaccgagucggugcU*U*U*U (SEQ ID NO: 304) | CACCACCCCGCGGGACTAGA (GGG) (SEQ ID NO: 341) CACCACCCCGCGGGACTAGA (SEQ ID NO: 342) |
| REG1-Z02 sgRNA (EX2_T2) spacer | CACCACCCCGCGGGACUA GA (SEQ ID NO: 305) | C*A*C*CACCCCGCGGGACUAG A (SEQ ID NO: 306) | |
| REG1-Z03 sgRNA (EX2_T3) | GGUCUGGCGCUCCCGCUCGGguuuuagagcuagaaauagcaaguuaaaauaaggcuaguccguuaucaacuugaaaaaguggcaccgagucggugcUUUU (SEQ ID NO: 307) | G*G*U*CUGGCGCUCCCGCUCGGguuuuagagcuagaaauagcaaguuaaaauaaggcuaguccguuaucaacuugaaaaaguggcaccgagucggugcU*U*U*U (SEQ ID NO: 308) | GGTCTGGCGCTCCCGCTCGG (TGG) (SEQ ID NO: 343) GGTCTGGCGCTCCCGCTCGG (SEQ ID NO: 344) |
| REG1-Z03 sgRNA (EX2_T3) spacer | GGUCUGGCGCUCCCGCUCGG (SEQ ID NO: 309) | G*G*U*CUGGCGCUCCCGCUCGG (SEQ ID NO: 310) | |
| REG1-Z04 sgRNA (EX4_T1) | UUCACACCAUCACGACGCGUguuuuagagcuagaaauagcaaguuaaaauaaggcuaguccguuaucaacuugaaaaaguggcaccgagucggugcUUUU (SEQ ID NO: 311) | U*U*C*ACACCAUCACGACGCGUguuuuagagcuagaaauagcaaguuaaaauaaggcuaguccguuaucaacuugaaaaaguggcaccgagucggugcU*U*U*U (SEQ ID NO: 312) | TTCACACCATCACGACGCGT (GGG) (SEQ ID NO: 345) TTCACACCATCACGACGCGT (SEQ ID NO: 346) |
| REG1-Z04 sgRNA (EX4_T1) spacer | UUCACACCAUCACGACGCGU (SEQ ID NO: 313) | U*U*C*ACACCAUCACGACGCGU (SEQ ID NO: 314) | |
| REG1-Z05 sgRNA (EX4_T2) | ACACCAUCACGACGCGUGGGguuuuagagcuagaaauagcaaguuaaaauaaggcuaguccguuaucaacuugaaaaaguggcaccgagucggugcUUUU (SEQ ID NO: 315) | A*C*A*CCAUCACGACGCGUGGGguuuuagagcuagaaauagcaaguuaaaauaaggcuaguccguuaucaacuugaaaaaguggcaccgagucggugcU*U*U*U (SEQ ID NO: 316) | ACACCATCACGACGCGTGGG (TGG) (SEQ ID NO: 347) ACACCATCACGACGCGTGGG (SEQ ID NO: 348) |
| REG1-Z05 sgRNA (EX4_T2) spacer | ACACCAUCACGACGCGUGGG (SEQ ID NO: 317) | A*C*A*CCAUCACGACGCGUGGG (SEQ ID NO: 318) | |
| REG1-Z06 | CUACGAGUCUGACGGGAU | C*U*A*CGAGUCUGACGGGAUC | CTACGAGTCTGACGGGATCG |
| Name | Unmodified Sequence | Modified Sequence | Target Sequences (PAM) |
| sgRNA (EX4_T3) | CGguuuuagagcuagaaauagcaaguuaaaauaaggcuaguccguuaucaacuugaaaaaguggcaccgagucggugcUUUU (SEQ ID NO: 319) | GguuuuagagcuagaaauagcaaguuaaaauaaggcuaguccguuaucaacuugaaaaaguggcaccgagucggugcU*U*U*U (SEQ ID NO: 320) | (TGG) (SEQ ID NO: 349) CTACGAGTCTGACGGGATCG (SEQ ID NO: 350) |
| REG1-Z06 sgRNA (EX4_T3) spacer | CUACGAGUCUGACGGGAUCG (SEQ ID NO: 321) | C*U*A*CGAGUCUGACGGGAUCG (SEQ ID NO: 322) | |
| REG1-Z07 sgRNA (EX4_T4) | UUGCCACCCACGCGUCGUGAguuuuagagcuagaaauagcaaguuaaaauaaggcuaguccguuaucaacuugaaaaaguggcaccgagucggugcUUUU (SEQ ID NO: 323) | U*U*G*CCACCCACGCGUCGUGAguuuuagagcuagaaauagcaaguuaaaauaaggcuaguccguuaucaacuugaaaaaguggcaccgagucggugcU*U*U*U (SEQ ID NO: 324) | TTGCCACCCACGCGTCGTGA (TGG) (SEQ ID NO: 351) TTGCCACCCACGCGTCGTGA (SEQ ID NO: 352) |
| REG1-Z07 sgRNA (EX4_T4) spacer | UUGCCACCCACGCGUCGUGA (SEQ ID NO: 325) | U*U*G*CCACCCACGCGUCGUGA (SEQ ID NO: 326) | |
| REG1-Z08 sgRNA (EX4_T5) | GUUCACACCAUCACGACGCGguuuuagagcuagaaauagcaaguuaaaauaaggcuaguccguuaucaacuugaaaaaguggcaccgagucggugcUUUU (SEQ ID NO: 327) | G*U*U*CACACCAUCACGACGCGguuuuagagcuagaaauagcaaguuaaaauaaggcuaguccguuaucaacuugaaaaaguggcaccgagucggugcU*U*U*U (SEQ ID NO: 328) | GTTCACACCATCACGACGCG (TGG) (SEQ ID NO: 353) GTTCACACCATCACGACGCG (SEQ ID NO: 354) |
| REG1-Z08 sgRNA (EX4_T5) spacer | GUUCACACCAUCACGACGCG (SEQ ID NO: 329) | G*U*U*CACACCAUCACGACGCG (SEQ ID NO: 330) | |
| REG1-Z09 sgRNA (EX4_T6) | CACGAUCCCGUCAGACUCGUguuuuagagcuagaaauagcaaguuaaaauaaggcuaguccguuaucaacuugaaaaaguggcaccgagucggugcUUUU (SEQ ID NO: 331) | C*A*C*GAUCCCGUCAGACUCGUguuuuagagcuagaaauagcaaguuaaaauaaggcuaguccguuaucaacuugaaaaaguggcaccgagucggugcU*U*U*U (SEQ ID NO: 332) | CACGATCCCGTCAGACTCGT (AGG) (SEQ ID NO: 355) CACGATCCCGTCAGACTCGT (SEQ ID NO: 356) |
| REG1-Z09 sgRNA (EX4_T6) spacer | CACGAUCCCGUCAGACUCGU (SEQ ID NO: 333) | C*A*C*GAUCCCGUCAGACUCGU (SEQ ID NO: 334) | |
| REG1-Z10 sgRNA (EX4_T7) | ACGACGCGUGGGUGGCAAGCguuuuagagcuagaaauagcaaguuaaaauaaggcuaguccguuaucaacuugaaaaaguggcaccgagucggugcUUUU (SEQ ID NO: 335) | A*C*G*ACGCGUGGGUGGCAAGCguuuuagagcuagaaauagcaaguuaaaauaaggcuaguccguuaucaacuugaaaaaguggcaccgagucggugcU*U*U*U (SEQ ID NO: 336) | ACGACGCGTGGGTGGCAAGC (GGG) (SEQ ID NO: 357) ACGACGCGTGGGTGGCAAGC (SEQ ID NO: 358) |
| REG1-Z10 sgRNA (EX4_T7) spacer | ACGACGCGUGGGUGGCAAGC (SEQ ID NO: 337) | A*C*G*ACGCGUGGGUGGCAAGC (SEQ ID NO: 338) | |

* indicates a nucleotide with a 2′-O-methyl phosphorothioate modification.

TABLE 21

TGFBRII gRNA Sequences/Target Sequences

| Name | Unmodified Sequence | Modified Sequence | Target Sequence (PAM) |
|---|---|---|---|
| TGFBRII sgRNA (EX1_T1) | CCGACUUCUGAACGUGCGGUguuuuagagcuagaaauagcaaguuaaaauaaggcuaguccguuaucaacuugaaaaaguggcaccgagucggugcUUUU (SEQ ID NO: 359) | C*C*G*ACUUCUGAACGUGCGGUGGGguuuuagagcuagaaauagcaaguuaaaauaaggcuaguccguuaucaacuugaaaaaguggcaccgagucggugcU*U*U*U (SEQ ID NO: 360) | CCGACTTCTGAACGTGCGGT (GGG) (SEQ ID NO: 395) CCGACTTCTGAACGTGCGGT (SEQ ID NO: 396) |
| TGFBRII sgRNA (EX1_T1) spacer | CCGACUUCUGAACGUGCGGU (SEQ ID NO: 361) | C*C*C*GACUUCUGAACGUGCGGU (SEQ ID NO: 362) | |
| TGFBRII sgRNA (EX1_T2) | UGCUGGCGAUACGCGUCCACguuuuagagcuagaaauagcaaguuaaaauaaggcuaguccguuaucaacuugaaaaaguggcaccgagucggugcUUUU (SEQ ID NO: 363) | U*G*C*UGGCGAUACGCGUCCACguuuuagagcuagaaauagcaaguuaaaauaaggcuaguccguuaucaacuugaaaaaguggcaccgagucggugcU*U*U*U (SEQ ID NO: 364) | TGCTGGCGATACGCGTCCAC (AGG) (SEQ ID NO: 397) TGCTGGCGATACGCGTCCAC (SEQ ID NO: 398) |
| TGFBRII sgRNA (EX1_T2) spacer | UGCUGGCGAUACGCGUCCAC (SEQ ID NO: 365) | U*G*C*UGGCGAUACGCGUCCAC (SEQ ID NO: 366) | |
| TGFBRII sgRNA (EX1_T3) | UCGGUCUAUGACGAGCAGCGguuuuagagcuagaaauagcaaguuaaaauaaggcuaguccguuaucaacuugaaaaaguggcaccgagucggugcUUUU (SEQ ID NO: 367) | U*C*G*GUCUAUGACGAGCAGCGguuuuagagcuagaaauagcaaguuaaaauaaggcuaguccguuaucaacuugaaaaaguggcaccgagucggugcU*U*U*U (SEQ ID NO: 368) | TCGGTCTATGACGAGCAGCG(GGG) (SEQ ID NO: 399) TCGGTCTATGACGAGCAGCG (SEQ ID NO: 400) |
| TGFBRII sgRNA (EX1_T3) spacer | UCGGUCUAUGACGAGCAGCG (SEQ ID NO: 369) | U*C*G*GUCUAUGACGAGCAGCG (SEQ ID NO: 370) | |
| TGFBRII sgRNA (EX2_T1) | AUGGGCAGUCCUAUUACAGCguuuuagagcuagaaauagcaaguuaaaauaaggcuaguccguuaucaacuugaaaaaguggcaccgagucggugcUUUU (SEQ ID NO: 371) | A*U*G*GGCAGUCCUAUUACAGCguuuuagagcuagaaauagcaaguuaaaauaaggcuaguccguuaucaacuugaaaaaguggcaccgagucggugcU*U*U*U (SEQ ID NO: 372) | ATGGGCAGTCCTATTACAGC (TGG) (SEQ ID NO: 401) ATGGGCAGTCCTATTACAGC (SEQ ID NO: 402) |
| TGFBRII sgRNA (EX2_T1) spacer | AUGGGCAGUCCUAUUACAGC (SEQ ID NO: 373) | A*U*G*GGCAGUCCUAUUACAGC (SEQ ID NO: 374) | |
| TGFBRII sgRNA (EX3_T1) | AUUGUUCACUUGUUAGCCCCguuuuagagcuagaaauagcaaguuaaaauaaggcuaguccguuaucaacuugaaaaaguggcaccgagucggugcUUUU (SEQ ID NO: 375) | A*U*U*GUUCACUUGUUAGCCCCAGGguuuuagagcuagaaauagcaaguuaaaauaaggcuaguccguuaucaacuugaaaaaguggcaccgagucggugcU*U*U*U (SEQ ID NO: 376) | ATTGTTCACTTGTTAGCCCC (AGG) (SEQ ID NO: 403)ATTGTTCACTTGTTAGCCCC (SEQ ID NO: 404) |
| TGFBRII sgRNA (EX3_T1) spacer | AUUGUUCACUUGUUAGCCCC (SEQ ID NO: 377) | A*U*U*GUUCACUUGUUAGCCCC (SEQ ID NO: 378) | |
| TGFBRII sgRNA (EX3_T2) | GCUGAAGAACUGCCUCUAUAguuuuagagcuagaaauagcaaguuaaaauaaggcuaguccguuaucaacuugaaaaaguggcaccgagucggugcUUUU (SEQ ID NO: 379) | G*C*U*GAAGAACUGCCUCUAUAguuuuagagcuagaaauagcaaguuaaaauaaggcuaguccguuaucaacuugaaaaaguggcaccgagucggugcU*U*U*U (SEQ ID NO: 380) | GCTGAAGAACTGCCTCTATA(TGG) (SEQ ID NO: 405) GCTGAAGAACTGCCTCTATA (SEQ ID NO: 406) |
| TGFBRII sgRNA (EX3_T2) spacer | GCUGAAGAACUGCCUCUAUA (SEQ ID NO: 381) | G*C*U*GAAGAACUGCCUCUAUA (SEQ ID NO: 382) | |
| TGFBRII sgRNA (EX4_T1) | GCAGGAUUUCUGGUUGUCACguuuuagagcuagaaauagcaaguuaaaauaaggcuaguccguuaucaacuugaaaaaguggcaccgagucggugcUUUU (SEQ ID NO: 383) | G*C*A*GGAUUUCUGGUUGUCACguuuuagagcuagaaauagcaaguuaaaauaaggcuaguccguuaucaacuugaaaaaguggcaccgagucggugcU*U*U*U (SEQ ID NO: 384) | GCAGGATTTCTGGTTGTCAC (AGG) (SEQ ID NO: 407) GCAGGATTTCTGGTTGTCAC (SEQ ID NO: 408) |
| TGFBRII sgRNA (EX4_T1) spacer | GCAGGAUUUCUGGUUGUCAC (SEQ ID NO: 385) | G*C*A*GGAUUUCUGGUUGUCAC (SEQ ID NO: 386) | |
| TGFBRII sgRNA (EX4_T2) | CUCCAUCUGUGAGAAGCCACguuuuagagcuagaaauagcaaguuaaaauaaggcuaguccguuaucaacuugaaaaaguggcaccgagucggugcUUUU (SEQ ID NO: 387) | C*U*C*CAUCUGUGAGAAGCCACguuuuagagcuagaaauagcaaguuaaaauaaggcuaguccguuaucaacuugaaaaaguggcaccgagucggugcU*U*U*U (SEQ ID NO: 388) | CTCCATCTGTGAGAAGCCAC (AGG) (SEQ ID NO: 409) CTCCATCTGTGAGAAGCCAC (SEQ ID NO: 410) |
| TGFBRII sgRNA (EX4_T2) spacer | CUCCAUCUGUGAGAAGCCAC (SEQ ID NO: 389) | C*U*C*CAUCUGUGAGAAGCCAC (SEQ ID NO: 390) | |
| TGFBRII sgRNA (EX5_T1) | CCCCUACCAUGACUUUAUUCguuuuagagcuagaaauagcaaguuaaaauaaggcuaguccguuaucaacuugaaaaaguggcaccgagucggugcUUUU (SEQ ID NO: 391) | C*C*C*CUACCAUGACUUUAUUCguuuuagagcuagaaauagcaaguuaaaauaaggcuaguccguuaucaacuugaaaaaguggcaccgagucggugcU*U*U*U (SEQ ID NO: 392) | CCCCTACCATGACTTTATTC (TGG) (SEQ ID NO: 411) CCCCTACCATGACTTTATTC (SEQ ID NO: 412) |
| TGFBRII sgRNA (EX5_T1) spacer | CCCCUACCAUGACUUUAUUC (SEQ ID NO: 393) | C*C*C*CUACCAUGACUUUAUUC (SEQ ID NO: 394) | |

*: 2′-O-methyl phosphorothioate residue

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ± 20 %, preferably up to ± 10 %, more preferably up to ± 5 %, and more preferably still up to ± 1 % of a given value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A population of genetically engineered T cells, comprising:
a disrupted Casitas B-lineage lymphoma proto-oncogene-B (cbl-b).

2. The population of genetically engineered T cells of claim 1, wherein the T cells are further engineered to express a chimeric antigen receptor (CAR).

3. The population of genetically engineered T cells of claim 1 or claim 2, wherein the disrupted cbl-b gene is genetically edited in exon 2, exon 7, exon 9, exon 11, or exon 12, optionally wherein the disrupted cbl-b gene is genetically edited in exon 2, exon 7, or exon 9.

4. The population of genetically engineered T cells of any one of claims 1-3, wherein the disrupted cbl-b gene is genetically edited by a CRISPR/Cas-mediated gene editing system.

5. The population of genetically engineered T cells of claim 4, wherein the CRISPR/Cas-mediated gene editing system comprises a guide RNA (gRNA) targeting a site in the cbl-b gene that comprises a nucleotide sequence selected from the group consisting of SEQ ID NOs: 84, 86, 88, 90, 92, 94, 96, 98, 100, 102, 104, 106, 108, 110, and 112; optionally SEQ ID NO: 88, 92, 96, 104, or 106.

6. The population of genetically engineered T cells of claim 5, wherein the gRNA comprises a spacer having a nucleotide sequence selected from the group consisting of SEQ ID NOs: 25, 29, 33, 37, 41, 45, 49, 53, 57, 61, 65, 69, 73, 77, and 81, optionally SEQ ID NO: 33, 39, 49, 61, or 65.

7. The population of genetically engineered T cells of claim 6, wherein the gRNA further comprises a scaffold sequence.

8. The population of genetically engineered T cells of claim 7, wherein the gRNA comprises a nucleotide sequence selected from the group consisting of 23, 27, 31, 35, 39, 43, 47, 51, 55, 59, 63, 67, 71, 75, and 79, optionally SEQ ID NO: 31, 39, 47, 67, or 71.

9. The population of genetically engineered T cells of any one of claims 1-8, wherein the T cells further comprise a disrupted T cell receptor alpha chain constant region (TRAC) gene, a disrupted beta-2-microglobulin (β2M) gene, a disrupted CD70 gene, or a combination thereof.

10. The population of genetically engineered T cells of claim 9, wherein the T cells further comprise a disrupted TRAC gene, which has a deleted fragment comprising AGAGCAACAGTGCTGTGGCC (SEQ ID NO: 14).

11. The population of genetically engineered T cells of any one of claims 1-10, wherein the T cells further comprise a disrupted Regnase-1 (Reg1) gene, a disrupted Transforming Growth Factor Beta Receptor II (TGFBRII) gene, or a combination thereof.

12. The population of genetically engineered T cells of any one of claims 2-11, wherein the T cells comprise a nucleic acid encoding the CAR.

13. The population of genetically engineered T cells of claim 12, wherein the nucleic acid encoding the CAR is inserted in the genome of the T cells.

14. The population of genetically engineered T cells of claim 13, wherein the T cells comprise the disrupted TRAC gene, which comprises the nucleic acid encoding the CAR.

15. The population of genetically engineered T cells of claim 14, wherein the nucleic acid encoding the CAR replaces the deleted fragment in the disrupted TRAC gene.

16. The population of genetically engineered T cells of any one of claims 9-15, wherein the disrupted TRAC gene, the disrupted β2M gene, and/or the disrupted CD70 gene, is genetically edited by a CRISPR/Cas-mediated gene editing system.

17. The population of genetically engineered T cells of any one of claims 9-16, wherein the disrupted Reg1 gene and/or the disrupted TGFBRII gene is genetically edited by a CRISPR/Cas-mediated gene editing system.

18. The population of genetically engineered T cells of claim 16 or claim 17, wherein the disrupted TRAC gene is genetically edited by a CRISPR/Cas-mediated gene editing system, which comprises a gRNA comprising the nucleotide sequence of SEQ ID NO: 3.

19. The population of genetically engineered T cells of any one of claims 16-18, wherein the disrupted β2M gene is genetically edited by a CRISPR/Cas-mediated gene editing system, which comprises a gRNA comprising the nucleotide sequence of SEQ ID NO: 7.

20. The population of genetically engineered T cells of any one of claims 16-19, wherein the disrupted CD70 gene is genetically edited by a CRISPR/Cas-mediated gene editing system, which comprises a gRNA comprising the nucleotide sequence of SEQ ID NO: 11.

21. The population of genetically engineered T cells of any one of claims 16-20, wherein the disrupted Reg1 gene is genetically edited by a CRISPR/Cas-mediated gene editing system, which comprises a gRNA comprising the nucleotide sequence of SEQ ID NO: 337.

22. The population of genetically engineered T cells of any one of claims 16-21, wherein the disrupted TGFBRII gene is genetically edited by a CRISPR/Cas-mediated gene editing system, which comprises a gRNA comprising the nucleotide sequence of SEQ ID NO: 393.

23. The population of genetically engineered T cells of claims 2-22, wherein the CAR comprises an extracellular antigen binding domain specific to a tumor antigen, a co-stimulatory signaling domain of 4-1BB or CD28, and a cytoplasmic signaling domain of CD3ζ.

24. The population of genetically engineered T cells of claim 23, wherein the tumor antigen is B-cell maturation antigen (BCMA), or CD70.

25. The population of genetically engineered T cells of claim 24, wherein the extracellular antigen binding domain is a single chain variable fragment (scFv) that binds BCMA, and optionally wherein the scFv comprises the amino acid sequence of SEQ ID NO: 277.

26. The population of genetically engineered T cells of claim 25, wherein the CAR comprises the amino acid sequence of SEQ ID NO: 274 or 275.

27. The population of genetically engineered T cells of claim 23, wherein the extracellular antigen binding domain is a single chain variable fragment (scFv) that binds CD70, and optionally wherein the scFv comprises the amino acid sequence of SEQ ID NO: 268 or 270.

28. The population of genetically engineered T cells of claim 27, wherein the CAR comprises the nucleotide sequence of SEQ ID NO: 265 or 266.

29. The population of genetically engineered T cells of any one of claims 23-28, wherein the genetically engineered T cells comprise the disrupted TRAC gene, the disrupted β2M gene, and the disrupted CD70 gene.

30. The population of genetically engineered T cells of any one of claims 23-29, wherein the T cells further comprise a disrupted Regnase-1 (Reg1) gene and a disrupted Transforming Growth Factor Beta Receptor II (TGFBRII) gene.

31. The population of genetically engineered T cells of any one of claims 1-30, wherein the genetically engineered T cells are derived from primary T cells of one or more human donors.

32. The population of genetically engineered T cells of any one of claims 1-31, wherein the genetically engineered T cells show cytokine-dependent growth.

33. The population of genetically engineered T cells of any one of claims 1-32, wherein the population of genetically engineered T cells has enhanced cytotoxicity and/or persistence as compared to non-engineered T cell counterparts.

34. A method for preparing the population of genetically engineered T cells of claim 1, the method comprising:
(a) providing a plurality of cells, which are T cells or precursor cells thereof;
(b) genetically editing a cbl-b gene of the T cells or the precursor cells thereof; and
(c) producing the population of genetically engineered T cells having a disrupted cbl-b gene.

35. The method of claim 34, wherein step (b) is performed by delivering to the plurality of cells an RNA-guided nuclease and a gRNA targeting the cbl-b gene.

36. The method of claim 35, wherein the gRNA targeting the cbl-b gene is specific to an exon of the cbl-b gene selected from the group consisting of exon 2, exon 7, exon 9, exon 11, and exon 12, optionally wherein the gRNA targeting the cbl-b gene is specific to exon 2.

37. The method of claim 36, wherein the gRNA targeting the cbl-b gene comprises a spacer having a nucleotide sequence selected from the group consisting of SEQ ID NOs: 25, 29, 33, 37, 41, 45, 49, 53, 57, 61, 65, 69, 73, 77, and 81, optionally SEQ ID NO: 33, 39, 49, 61, or 65.

38. The method of claim 37, wherein the gRNA further comprises a scaffold sequence.

39. The method of claim 38, wherein the gRNA comprises a nucleotide sequence selected from the group consisting of SEQ ID NOs: 23, 27, 31, 35, 39, 43, 47, 51, 55, 59, 63, 67, 71, 75, and 79, optionally SEQ ID NO: 31, 39, 47, 67, or 71.

40. The method of any one of claims 34-39, wherein the plurality of T cells in step (a) comprises one or more of the following genetic modifications:
(i) engineered to express a chimeric antigen receptor (CAR);
(ii) has a disrupted T cell receptor alpha chain constant region (TRAC) gene;
(iii) has a disrupted β2M gene; and
(iv) has a disrupted CD70 gene.

41. The method of any one of claims 34-40, wherein the plurality of T cells in step (a) comprises one or more of the following genetic modifications:
(v) has a disrupted Reg1 gene; and
(vi) has a disrupted TGFBRII gene.

42. The method of any one of claims 34-39, wherein the method further comprises:
(i) delivering to the T cells a nucleic acid encoding a chimeric antigen receptor (CAR);
(ii) genetically editing a TRAC gene to disrupt its expression;
(iii) genetically editing a β2M gene to disrupt its expression;
(iv) genetically editing a CD70 gene to disrupt its expression; or
(vi) a combination thereof.

43. The method of claim 42, wherein one or more of (ii)-(v) are performed by one or more CRISPR/Cas-mediated gene editing systems comprising one or more RNA-guided nucleases and one or more gRNAs targeting the TRAC gene, the β2M gene, and/or the CD70 gene.

44. The method of claim 43, wherein the gRNA targeting the TRAC gene comprises the nucleotide sequence of SEQ ID NO: 3.

45. The method of claim 43 or claim 44, wherein the gRNA targeting the β2M gene comprises the nucleotide sequence of SEQ ID NO: 7.

46. The method of any one of claims 42-45, wherein the gRNA targeting the CD70 gene comprises the nucleotide sequence of SEQ ID NO: 11.

47. The method of any one of claims 34-46, wherein the method further comprises:
(vi) genetically editing a Reg1 gene to disrupt its expression;
(vii) genetically editing a TGFBRII gene to disrupt its expression; or
(viii) a combination thereof.

48. The method of claim 47, wherein (vi) and/or (vii) are performed by one or more CRISPR/Cas-mediated gene editing systems comprising one or more RNA-guided nucleases and one or more gRNAs targeting the Reg1 gene and/or the TGFBRII gene.

49. The method of claim 47 or claim 48, wherein the gRNA targeting the Reg1 gene comprises the nucleotide sequence of SEQ ID NO: 337.

50. The method of any one of claims 47-49, wherein the gRNA targeting the TGFBRII gene comprises the nucleotide sequence of SEQ ID NO: 393.

51. The method of any one of claims 34-50, wherein the method comprises delivering to the T cells or the precursor cells thereof one or more ribonucleoprotein particles (RNP), which comprises the RNA-guided nuclease, and one or more of the gRNAs.

52. The method of any one of claims 34-51, wherein the RNA-guided nuclease is a Cas9 nuclease.

53. The method of claim 52, wherein the Cas9 nuclease is a S pyogenes Cas9 nuclease.

54. The method of any one of claims 42-53, wherein the nucleic acid encoding the CAR is in an AAV vector.

55. The method of any one of claims 42-54, wherein the nucleic acid encoding the CAR comprises a left homology arm and a right homology arm flanking the nucleotide sequence encoding the CAR; and wherein the left homology arm and the right homology arm are homologous to a genomic locus in the T cells, allowing for insertion of the nucleic acid into the genomic locus.

56. The method of claim 55, wherein the genomic locus is in the TRAC gene.

57. The method of claim 56, wherein the method comprising disrupting the TRAC gene by a CRISPR/Cas-mediated gene editing system comprising the gRNA that comprises the nucleotide sequence of SEQ ID NO: 3 and the nucleic acid encoding the CAR is inserted at the site targeted by the gRNA.

58. The method of any one of claims 34-57, wherein the method comprising delivering to the T cells a nucleic acid encoding a CAR, which is specific to CD70, and genetically editing the CD70 gene to disrupt its expression.

59. The method of any one of claims 34-58, wherein the T cells of step (a) are derived from primary T cells of one or more human donors.

60. A population of genetically engineered T cells, which is prepared by a method of any one of claims 34-59.

61. A method for eliminating undesired cells in a subject, the method comprising administering to a subject in need thereof genetically engineered T cells expressing a disrupted cbl-b gene and a chimeric antigen receptor targeting the undesired cells.

62. The method of claim 54, wherein the genetically engineered T cells are set forth in any one of claims 2-33 and 60.

63. The method of claim 61 or claim 62, wherein the undesired cells are cancer cells.

64. The method of any one of claims 61-63, wherein the subject is a human patient suffering from a hematologic cancer or a solid tumor.

65. A guide RNA (gRNA) targeting a cbl-b gene, comprising a nucleotide sequence specific to a fragment in exon 2, exon 7, exon 9, exon 11, or exon 12 of the cbl-b gene, optionally wherein the gRNA comprises a nucleotide sequence specific to exon 2, exon 7, or exon 9 of the cbl-b gene.

66. The gRNA of claim 65, wherein the gRNA comprises a spacer having the nucleotide sequence selected from the group consisting of SEQ ID NOs: SEQ ID NOs: 25, 29, 33, 37, 41, 45, 49, 53, 57, 61, 65, 69, 73, 77, and 81, optionally SEQ ID NO: 33, 39, 49, 61, or 65.

67. The gRNA of claim 66, wherein the gRNA further comprises a scaffold sequence.

68. The gRNA of any one of claims 65-67, wherein the gRNA comprises one or more modified nucleotides.

69. The gRNA of claim 68, wherein the gRNA comprises one or more 2′-O-methyl phosphorothioate residues at the 5′ and/or 3′ terminus of the gRNA.

70. The gRNA of claim 59, which comprises the nucleotide sequence of any one of SEQ ID NO: SEQ ID NOs: 24, 28, 32, 36, 40, 44, 48, 52, 56, 60, 64, 68, 72, 76, and 80, optionally SEQ ID NO: 31, 39, 47, 67, or 71.
